SEQUENCE LISTING (57 sequences)

SEQ ID NO: 1 - RNA, 7 nt, Artificial Sequence
  Synthetic
gggaccc

SEQ ID NO: 2 - RNA, 22 nt, Artificial Sequence
  Synthetic
gggacccggggagagauguaag

SEQ ID NO: 3 - protein, 15 aa, Artificial Sequence
  Synthetic
FRNELAAAVATFGQL

SEQ ID NO: 4 - protein, 15 aa, Artificial Sequence
  Synthetic
FRNELAYPVLTFGQL

SEQ ID NO: 5 - RNA, 22 nt, Artificial Sequence
  Synthetic
gggacccggggagagauguaag

SEQ ID NO: 6 - RNA, 22 nt, Artificial Sequence
  Synthetic
uagcuuaucagacugauguuga

SEQ ID NO: 7 - RNA, 23 nt, Artificial Sequence
  Synthetic
uuaaugcuaauugugauaggggu

SEQ ID NO: 8 - RNA, 24 nt, Artificial Sequence
  Synthetic
gggggcagggccuuugugaaggcg

SEQ ID NO: 9 - RNA, 24 nt, Artificial Sequence
  Synthetic
gggacccagggagagagacguaag

SEQ ID NO: 10 - RNA, 22 nt, Artificial Sequence
  Synthetic
agacacauuuggagagggaccc

SEQ ID NO: 11 - RNA, 22 nt, Artificial Sequence
  Synthetic
aaaacccggggagagauguaag

SEQ ID NO: 12 - RNA, 22 nt, Artificial Sequence
  Synthetic
gggaaaaggggagagauguaag

SEQ ID NO: 13 - RNA, 22 nt, Artificial Sequence
  Synthetic
gggacccgaaaagagauguaag

SEQ ID NO: 14 - RNA, 22 nt, Artificial Sequence
  Synthetic
gggacccggggaaaaauguaag

SEQ ID NO: 15 - RNA, 22 nt, Artificial Sequence
  Synthetic
gggacccggggagagauaaaag

SEQ ID NO: 16 - RNA, 22 nt, Artificial Sequence
  Synthetic
aaaaaaaggggagagauguaag

SEQ ID NO: 17 - RNA, 7 nt, Artificial Sequence
  Synthetic
aaaaaaa

SEQ ID NO: 18 - RNA, 22 nt, Artificial Sequence
  Synthetic
gggacccggggagagauguaag

SEQ ID NO: 19 - RNA, 22 nt, Artificial Sequence
  Synthetic
aaaaaaaggggagagauguaag

SEQ ID NO: 20 - RNA, 22 nt, Artificial Sequence
  Synthetic
gggacccggggagagauguaag

SEQ ID NO: 21 - RNA, 22 nt, Artificial Sequence
  Synthetic
aaaaaaaggggagagauguaag

SEQ ID NO: 22 - RNA, 22 nt, Artificial Sequence
  Synthetic
cuuacgucucucccuggguccc

SEQ ID NO: 23 - DNA, 20 bp, Artificial Sequence
  Synthetic
cttacgtctctccctgggtc

SEQ ID NO: 24 - DNA, 22 bp, Artificial Sequence
  Synthetic
gtgtaacacgtctatacgccca

SEQ ID NO: 25 - DNA, 39 bp, Artificial Sequence
  Primer
agcctgggaggaccttctagaatgaagcgcggcttgagg

SEQ ID NO: 26 - DNA, 41 bp, Artificial Sequence
  Primer
ctcaccctgaagttctcaggatccctaaaagtccgggtggc

SEQ ID NO: 27 - DNA, 41 bp, Artificial Sequence; identical to SEQ ID NO: 26
  Primer

SEQ ID NO: 28 - DNA, 29 bp, Artificial Sequence
  Primer
gctgcagcagccgctggaactagtagtac

SEQ ID NO: 29 - DNA, 20 bp, Artificial Sequence
  Primer
agaattgaaggccattccag

SEQ ID NO: 30 - DNA, 29 bp, Artificial Sequence
  Primer
aattctgctggaataatcgctggaactag

SEQ ID NO: 31 - DNA, 24 bp, Artificial Sequence
  Primer
attattccagtagaattgaaggcc

SEQ ID NO: 32 - DNA, 35 bp, Artificial Sequence
  Primer
atgaggcagcaatagacgctctgaattcatttcca

SEQ ID NO: 33 - DNA, 27 bp, Artificial Sequence
  Primer
gagtactactagttccattgattattc

SEQ ID NO: 34 - DNA, 22 bp, Artificial Sequence
  Primer
tatatggcgtggcaatgtggag

SEQ ID NO: 35 - DNA, 22 bp, Artificial Sequence
  Primer
cgctgggatgttgaggaacaag

SEQ ID NO: 36 - DNA, 22 bp, Artificial Sequence
  Primer
gccccattgctttccttaatcc

SEQ ID NO: 37 - DNA, 22 bp, Artificial Sequence
  Primer
gctgaaggcatcttggaaattc

SEQ ID NO: 38 - DNA, 24 bp, Artificial Sequence
  Primer
agcaccgcattgctttccttaatc

SEQ ID NO: 39 - DNA, 19 bp, Artificial Sequence
  Primer
gaaggcatcttggaaattc

SEQ ID NO: 40 - DNA, 31 bp, Artificial Sequence
  Primer
gctgaggcggaatacgcagccctgacctttg

SEQ ID NO: 41 - DNA, 22 bp, Artificial Sequence
  Primer
gtttagagctgagttggcatac

SEQ ID NO: 42 - DNA, 22 bp, Artificial Sequence
  Primer
gtttagaaatgaggcggcatac

SEQ ID NO: 43 - DNA, 22 bp, Artificial Sequence
  Primer
aatggttctaggaaggcatctc

SEQ ID NO: 44 - DNA, 24 bp, Artificial Sequence
  Primer
aatgagttggaatacccagtcctg

SEQ ID NO: 45 - DNA, 24 bp, Artificial Sequence
  Primer
aatgagttggcatacgcagtcctg

SEQ ID NO: 46 - DNA, 24 bp, Artificial Sequence
  Primer
aatgagttggcatacccagccctg

SEQ ID NO: 47 - DNA, 22 bp, Artificial Sequence
  Primer
tctaaacaatggttctaggaag

SEQ ID NO: 48 - DNA, 32 bp, Artificial Sequence
  Primer
gttggcagccgcagtcgcgacctttgggcagc

SEQ ID NO: 49 - DNA, 23 bp, Artificial Sequence
  Primer
tcatttctaaacaatggttctag

SEQ ID NO: 50 - RNA, 24 nt, Artificial Sequence
  Synthetic
gggacccagggagagagacguaag

SEQ ID NO: 51 - RNA, 24 nt, Artificial Sequence
  Synthetic
gggacccagggagagagacguaag

SEQ ID NO: 52 - RNA, 24 nt, Artificial Sequence
  Synthetic
gggacccagggagagagacguaag

SEQ ID NO: 53 - RNA, 24 nt, Artificial Sequence
  Synthetic
gggacccggggagagagauguaag

SEQ ID NO: 54 - RNA, 24 nt, Artificial Sequence
  Synthetic
gggacccugggagagagauguaag

SEQ ID NO: 55 - protein, 242 aa, Homo sapiens
MMNLGSYCLGLIPMTILVVNIKPGMAFNSTGIINETSDHSEILDTTNSYLIKTCMILVFLSSIFGYCKEAGQIFQQKRNYFMDISNVLEWIIYTTGIIFVLPLFVEIPAHLQWQCGAIAVYFYWMNFLLYLQRFENCGIFIVMLEVILKTLLRSTVVFIFLLLAFGLSFYILLNLQDPFSSPLLSIIQTFSMMLGDINYRESFLEPYLRNELAHPVLSFAQLVSFTIFVPIVLMNLLIGLAV

SEQ ID NO: 56 - protein, 245 aa, Mus musculus
AHMMNLGSYCLGLIPMTLLVVKIQPGMAFNSTGIINGTSSTHEERIDTLNSFPIKICMILVFLSSIFGYCKEVIQIFQQKRNYFLDYNNALEWVIYTTSIIFVLPLFLNIPAYMQWQCGAIAIFFYWMNFLLYLQRFENCGIFIVMLEVIFKTLLRSTGVFIFLLLAFGLSFYVLLNFQDAFSTPLLSLIQTFSMMLGDINYRDAFLEPLFRNELAYPVLTFGQLIAFTMFVPIVLMNLLIGLAV

SEQ ID NO: 57 - protein, 245 aa, Rattus norvegicus
AHMMNLGSYCLGLIPMTLLVVKIQPGMAFNSTGIINETISTHEERINTLNSFPLKICMILVFLSSIFGYCKEVVQIFQQKRNYFLDYNNALEWVIYTTSMIFVLPLFLDIPAYMQWQCGAIAIFFYWMNFLLYLQRFENCGIFIVMLEVIFKTLLRSTGVFIFLLLAFGLSFYVLLNFQDAFSTPLLSLIQTFSMMLGDINYRDAFLEPLFRNELAYPVLTFGQLIAFTMFVPIVLMNLLIGLAV

(19) United States
(12) Patent Application Publication
(10) Pub. No.: US 2021/0283163 A1
(43) Pub. Date: Sep. 16, 2021

(54) COMPOSITIONS AND METHODS FOR THE TREATMENT OF ACUTE AND CHRONIC PRURITIS

(71) Applicant: Duke University, Durham, NC (US)

(72) Inventor: Ru-Rong Ji, Durham, NC (US)

(21) Appl. No.: 17/260,439

(22) PCT Filed: Jul. 18, 2019
(86) PCT No.: PCT/US2019/042479

§ 371 Date: Jan. 14, 2021

Related U.S. Application Data
(60) Provisional application No. 62/700,005, filed on Jul. 18, 2018.

(51) Int. Cl. A61K 31/7088; A61K 31/15; A61K 38/10; C12N 15/113

(52) U.S. Cl. CPC A61K 31/7088; A61K 31/15; C12N 2310/141; C12N 15/113; C12N 2310/113; A61K 38/10

(57) ABSTRACT

Disclosed herein are miR-711 inhibitors. The miR-711 inhibitors may disrupt the binding of miR-711 to TRPA1. Further provided are methods of treating a disease or condition in a subject, methods of inhibiting miR-711, and methods of inhibiting TRPA1 in a subject. The methods may include administering to the subject a miR-711 inhibitor.

### CROSS-REFERENCE TO RELATED APPLICATIONS

This application claims priority to U.S. Provisional Patent Application No. 62/700,005, filed Jul. 18, 2018, which is incorporated herein by reference in its entirety.

### STATEMENT REGARDING FEDERALLY SPONSORED RESEARCH

This invention was made with government support under grant DE17794 awarded by the National Institutes of Health. The government has certain rights in the invention.

### FIELD

This disclosure relates to miR-711 inhibitors, TRPA1 inhibitors, and method of using the same in the treatment of disorders such as pruritis.

### INTRODUCTION

Chronic pruritus, one of the main symptoms in dermatology, is often intractable and has a high impact on patient's quality of life. Beyond dermatologic disorders, chronic pruritus is associated with systemic, neurologic, as well as psychologic diseases. The pathogenesis of acute and chronic (>6 weeks duration) pruritus is complex and involves in the skin a network of resident (e.g., sensory neurons) and transient inflammatory cells (e.g., lymphocytes). In the skin, several classes of histamine-sensitive or histamine-insensitve C-fibers are involved in itch transmission. Specific receptors have been discovered on cutaneous and spinal neurons to be exclusively involved in the processing of pruritic signals. Chronic pruritus is notoriously difficult to treat. Hence, there is a need to new treatments and therapies of chronic and acute pruritus.

### SUMMARY

In an aspect, the disclosure relates to a method of treating a disease or condition in a subject. The method may include administering to the subject a miR-711 inhibitor.

In a further aspect, the disclosure relates to a method of inhibiting TRPA1 in a subject. The method may include administering to the subject a miR-711 inhibitor.

Another aspect of the disclosure provides a method of inhibiting miR-711 in a subject. The method may include administering to the subject a miR-711 inhibitor selected from a miR-711/TRPA1 interaction blocking peptide, a polynucleotide complementary to miR-711, or a combination thereof.

In some embodiments, the miR-711 inhibitor is selected from a miR-711/TRPA1 interaction blocking peptide, a polynucleotide complementary to miR-711, or a combination thereof. In some embodiments, the miR-711/TRPA1 interaction blocking peptide comprises a polypeptide having an amino acid sequence of SEQ ID NO: 3 (FRNELAAAVATFGQL). In some embodiments, the miR-711/TRPA1 interaction blocking peptide comprises a polypeptide having an amino acid sequence of SEQ ID NO: 4 (FRNELAYPVLTFGQL). In some embodiments, the miR-711 inhibitor comprises a polynucleotide complementary to miR-711 or a portion or fragment thereof. In some embodiments, the method further includes additionally administering a TRPA1 inhibitor. In some embodiments, the TRPA1 inhibitor is selected from HC030031 or A967079, or a pharmaceutically acceptable salt thereof. In some embodiments, the disease or condition is selected from pruritis, atopic eczema, and psoriasis. In some embodiments, the pruritis is chronic pruritis. In some embodiments, the pruritis is acute pruritis. In some embodiments, the pruritis is lymphoma-induced pruritis. In some embodiments, the pruritis is pruritis associated with lymphoma. In some embodiments, the pruritis is pruritis associated with liver disease. In some embodiments, miR-711 comprises a core polynucleotide sequence of SEQ ID NO: 1. In some embodiments, the miR-711 inhibitor inhibits nerve fibers expressing TRPA1. In some embodiments, the binding of miR-711 to the extracellular side of TRPA1 is inhibited. In some embodiments, the binding of miR-711 to TRPA1 at S5-S6 loop is inhibited. In some embodiments, the binding of miR-711 to TRPA1 at an amino acid corresponding to P934 of human TRPA1 (SEQ ID NO: 55) is inhibited.

Another aspect of the disclosure provides a composition comprising a miR-711 inhibitor, wherein the miR-711 inhibitor is selected from a miR-711/TRPA1 interaction blocking peptide, a polynucleotide complementary to miR-711, or a combination thereof. In some embodiments, the miR-711/TRPA1 interaction blocking peptide comprises a polypeptide having an amino acid sequence of SEQ ID NO: 3 (FRNELAAAVATFGQL) or SEQ ID NO: 4 (FRNELAYPVLTFGQL). In some embodiments, the composition further includes a TRPA1 inhibitor. In some embodiments, the TRPA1 inhibitor is selected from HC030031 or A967079, or a pharmaceutically acceptable salt thereof.

The disclosure provides for other aspects and embodiments that will be apparent in light of the following detailed description and accompanying figures.

### BRIEF DESCRIPTION OF THE DRAWINGS

FIG. 1A-FIG. 1J. Intradermal miR-711 Induces Itch but Not Pain via the GGGACCC Core Sequence and TRPA1. (FIG. 1A) Intradermal cheek injection of GGGACCC containing miRNAs (mmu-miR-711, has-miR-711, and has-miR-642b-3p), but not mmu-miR-21, mmu-miR-155, and mmu-miR-326, all at 1 mM (5 μL), induces scratching but not wiping in naive mice. Intradermal injection of AITC at a high concentration (10 mM, 5 μL) induces wiping but not scratching. \hat{ }\hat{ }\hat{ }p≤0.001, versus vehicle, ***p<0.001, one-way ANOVA, n=5-7 mice/group. (FIG. 1B) mmu-miR-711-induced scratching is reduced in Trpa1^{-/-} but not Trpv1^{-/-} and Tlr7^{-/-} mice. **p<0.01, versus WT, one-way ANOVA, n=5-6 mice/group. (FIG. 1C) Sequences of the miRNAs tested in this study. The core sequence of these miRNAs is highlighted in red. (FIG. 1D) Sequences of mmu-miR-711 and 6 mutants of mmu-miR-711 (m1 to m6). The mutated nucleotides are highlighted in red. (FIG. 1E and FIG. 1F) The core sequence GGGACCC is both required and sufficient for miR-711 to induce pruritus. (FIG. 1E) Scratching induced by intradermal injection of mmu-miR-711 and its mutants (1 mM, 5 μL). ***p<0.001, versus miR-711, one-way ANOVA, n=5-10 mice/group. (FIG. 1F) The core sequence GGGACCC but not mutant sequence AAAAAAA is sufficient to elicit scratching not wiping in naive mice. ***p<0.01, two-tailed Student's t test, n=5 mice/group. (FIG. 1G and FIG. 1H) Intraplantar injection of AITC (10 mM, 10 μL) but not miR-711 (1 mM, 10 μL) elicits heat hyperalgesia (FIG. 1G) and mechanical allodynia (FIG. 1H), ***p<0.001, two-way ANOVA, n=5 mice/group. (FIG. 1I and FIG. 1J) Intraplantar injection of AITC (5 mM, 10 μL) and capsaicin (1 mM, 10 μL) but not miR-711 (1 mM, 10 μL) induces neurogenic inflammation in a hindpaw, as measured by Evans blue test. (FIG. 1I) Images of hind paws with Evans blue staining. Ipsi, ipsilateral paws; Contra, contralateral paws. (FIG. 1J) Quantification of Evans blue staining in ipsilateral and contralateral hind paws. *p<0.05, two-tailed Student's t test, n=5-6 mice/group. Data are represented as mean±SEM. See also FIG. 9A-FIG. 9F.

FIG. 2A-FIG. 2K. miR-711 Activates TRPA1 in Heterologous Cells. (FIG. 2A-FIG. 2E) mmu-miR-711 induces inward currents in HEK293 cells expressing hTRPA1. (FIG. 2A) Traces of inward currents induced by mmu-miR-711 and the core sequence. Note that the induced currents are blocked by A967079 (10 μM). (FIG. 2B) Quantification (amplitude) of inward currents induced by AITC (50 μM), miRNAs (10 μM), core sequence, mutant RNA oligos (10 μM), and the effects of A967079 (10 and 50 μM). n=6-11 cells/group. (FIG. 2C) Dose-response curves comparing the amplitudes of inward currents induced by mmu-miR-711 and AITC. n=5-11 cells/group. (FIG. 2D) Latency of the inward currents evoked by AITC (50 μM) and mmu-miR-711 (10 μM) after bath perfusion. **p<0.01, two-tailed Student's t test, n=7-8 cells/group. (FIG. 2E) I/V curves elicited by mmu-miR-711 (10 μM), AITC (100 μM), and mmu-miR-711+A967079 (10 μM). n=5 cells for each condition. (FIG. 2F and FIG. 2G) mmu-miR-711 (10 μM) fails to evoke inward currents in CHO cells transfected with mouse Trpv1, Trpv2, Trpv3, and Trpv4 cDNAs. (FIG. 2F) Traces of inward currents induced by the agonists of TRPV1 (capsaicin, 50 nM), TRPV2 (cannabidiol, 100 μM), TRPV3 (carvacrol, 300 μM), and TRPV4 (GSK1016790A, 1 μM) but not by mmu-miR-711 (10 μM). (FIG. 2G) Quantification of inward currents described in (FIG. 2F). n=5-7 cells/group. (FIG. 2H and FIG. 2I) Single channel activities induced by bath application of AITC (50 μM) and mmu-miR-711 (10 μM) in outside-out patch recordings (held at −60 mV) in membrane excised from hTRPA1-expressing HEK293 cells. (FIG. 2H) Traces of single-channel activities. Left, schematic of outside-out patch recording. (FIG. 2I) Quantification of single channel open time (top) and open probability (bottom). *p<0.05, Two-tailed Student's t test, n=5 patches from 5 cells per group. (FIG. 2J and FIG. 2K) Single channel activities induced by bath application of AITC (50 μM) and mmu-miR-711 (10 μM) in inside-out patch recordings (held at −60 mV) in membrane excised from hTRPA1-expressing HEK293 cells. (FIG. 2J) Traces of single-channel activities. Left, schematic showing inside-out patch recording. (FIG. 2K) Quantification of single channel open time (top) and open probability (bottom). *P<0.05, **p<0.01, two-tailed Student's t test, n=6 patches from 6 cells per group. Data are represented as mean±SEM. See also FIG. 10A-FIG. 10E.

FIG. 3A-FIG. 3C. Calcium Imaging in DRG Cultures Showing Activation of a Subset of TRPA1-Expressing Sensory Neurons by miR-711 in Pirt-GCaMP3 Mice. (FIG. 3A) Representative images of calcium responses to mmu-miR-711 (50 μM), histamine (His, 500 μM), chloroquine (CQ, 1,000 μM), and AITC (200 μM) sequentially. Scale, 50 μm. (FIG. 3B) Representative traces show a neuronal calcium response to mmu-miR-711 (50 μM), Histamine (His, 500 μM), CQ (1,000 μM), and AITC (200 μM). (FIG. 3C) Venn diagram showing overlaps between miR-711-responsive neurons and histamine (His)-, CQ-, and AITC-responsive neurons and the percentage of each population in cultured DRG neurons. A total of 544 neurons from 3 mice were analyzed, and 11 neurons respond to all the stimuli. See also FIG. 11A-FIG. 11H.

FIG. 4A-FIG. 4E. miR-711 Induces Inward Currents and Action Potentials via TRPA1 in Mouse DRG Neurons. (FIG. 4A and FIG. 4B) Inward currents induced by miR-711 and AITC in small-diameter DRG neurons in WT and Trpa1^{-/-} mice. Cap, capsaicin. (FIG. 4A) Traces of inward currents. Note that miR-711-induced inward currents are blocked by A967079 (10 μM) and abolished in Trpa1^{-/-} mice. (FIG. 4B) Amplitude of inward currents. ***p<0.001, two-tailed Student's test, n=7-8 neurons/group. (FIG. 4C) Action potentials induced by miR-711 and AITC in small-diameter mouse DRG neurons in WT and Trpa1^{-/-} mice. Note that miR-711-induced action potentials are blocked by A967079 (10 μM) and abolished in Trpa1^{-/-} mice. n=10-15 neurons/group. Cap, capsaicin. (FIG. 4D and FIG. 4E) Distinct action potentials induced by miR-711 and AITC in small-diameter DRG neurons in WT mice. (FIG. 4D) Traces of the action potentials. Single action potentials in the red boxes are enlarged in the lower panels. (FIG. 4E) Quantification of the action potential's rising time or time to threshold, indicated as (1) in FIG. 4D and after hyperpolarization amplitude, indicated as (2) in FIG. 4D. *p<0.05, two-tailed Student's t test, n=7-9 neurons/group. Data are represented as mean±SEM. See also FIG. 12A-FIG. 12F.

FIG. 5A-FIG. 5G. Computer Simulation of miR-711 Core Sequence Binding to the Extracellular Loops of hTRPA1 and Identification of the Binding Sites. (FIG. 5A) Structure of the core sequence GGGACCC bound to hTRPA1 extracellular surface. The represented pose is the lowest estimated binding energy structure (i.e., −87 kcal/mol) extracted from the most populated cluster of high-affinity GGGACCC/TRPA1 conformations. The bound conformation of GGGACCC (labeled in orange) spans over three monomers of the channel, namely subunit 1, 2, and 3, as represented by green, cyan, and magenta cartoons, respectively. (FIG. 5B) Zoomed view of GGGACCC bound to TRPA1 extracellular surface. The hit map on TRPA1 surface represents the contact frequency between TRPA1 residues and GGGACCC in the most populated ensemble of high-affinity GGGACCC/TRPA1 conformations (i.e., estimated binding energy equal or lower than −75 kcal/mol). TRPA1 residues contacting GGGACCC with frequency of 100%, 97%-99%, and 70%-96% are revealed as red, orange, and yellow surface, respectively. All of the other TRPA1 residues are represented with a gray surface. Residues that upon mutation to alanine selectively disrupt the miRNA711-mediated activation of TRPA1 are represented as magenta surface. G001 and C007 indicate the first and last nucleotide of the core sequence, respectively. (FIG. 5C) Contact frequencies between hTRPA1 residues and GGGACCC. The frequencies of contacts are extracted from the most populated cluster of high-affinity GGGACCC/TRPA1 conformations. The TRPA1 residues that upon mutation to alanine selectively disrupt the GGGACCC-mediated activation of hTRPA1 are shown in red. (FIG. 5D) Distributions of GGGACCC estimated binding energies to hTRPA1 collected over a 2 million-step RexDMD simulation. The left shoulder of the distribution, starting at binding energy equal to −75 kcal/mol, characterizes the conformational ensemble of high-affinity GGGACCC/TRPA1 complex. (FIG. 5E) Schematic diagram of hTRPA1 with detailed residues for Subunit 1. Purple, red, and green residues indicate predicted but non-conservative Y936, predicted and ultra conservative P937, and non-predicted L939, respectively, on the S5-S6 loop. Black residues are non-mutated sites. (FIG. 5F and FIG. 5G) mmu-miR-711 and AITC induce inward currents in CHO cells transfected with wild-type and mutant mmu-Trpa1 cDNAs (M10, M11, M13). (FIG. 5F) Traces of inward currents induced by mmu-miR-711 on CHO cells expressing mmu-TRPA1 or its mutants. (FIG. 5G) Quantification of ATIC and mmu-miR-711 induced currents. ***p<0.001, one-way ANOVA, n=8-15 cells/group. Note that mutations M11 and M13 disrupt miR-711 but not AITC-induced currents. Data are represented as mean±SEM. See also FIG. 13A-FIG. 13K.

FIG. 6A-FIG. 6H. Disruption of miR-711/TRPA1 Interaction with a Blocking Peptide Reduces Itch. (FIGS. 6A-6D) Interaction between miR-711 and TRPA1. (FIG. 6A) RNA pull-down assay shows strong hTRPA1 binding to biotin (bio)-conjugated mmumiR-711 but weak hTRPA1 binding to bio-mmumiR-711 (m6). (FIG. 6B) RNA pull-down shows that wild-type mmumiR-711 (blue) but not mutant mmu-miR-711 (m6, red) competes with bio-mmu-miR-711 for the binding to hTRPA1. miR-711 (10-50 μM, blue) or mutant miR-711 (m6, 10-50 μM, red) were added 15 min before the incubation with biotin-conjugated miR-711 (10 μM). (FIG. 6C) Quantification of mmu-miR-711/hTRPA1 binding activity shown in FIG. 6B. \hat{ }\hat{ }p<0.01, \hat{ }\hat{ }\hat{ }p<0.001, versus control (no treatment), one-way ANOVA, *p<0.05, **p<0.01, #p<0.05, miR-711 versus m6, two-way ANOVA, n=5 experiments. (FIG. 6D) Live cell labeling shows the binding of Cy3-labeled mmu-miR-711 but not Cy3-labeled mmu-miR-711 (m6) to mTRPA1 on the surface of cultured DRG neurons. Scale, 20 μm. (FIGS. 6E-6H) A blocking peptide disrupts mmu-miR-711/hTRPA1 interaction and mmu-miR-711-induced currents and pruritus. (FIG. 6E) RNA pull-down assay showing disruption of the mmu-miR-711/hTRPA1 interaction by the blocking peptide but not by the mutated peptide. Right, quantification of binding. ***p<0.001, one-way ANOVA, n=4 cultures/group. (FIG. 6F) Representative traces showing the inhibition of the mmu-miR-711-induced inward currents by the blocking peptide but not the mutated peptide (25 μM) in hTRPA1-expressing HEK293 cells. (FIG. 6G) Quantification of the inward currents in (FIG. 6F). ***p<0.001, **p<0.01 versus vehicle, one-way ANOVA, n=5-10 cells/group. (FIG. 6H) The blocking peptide (2 mM, 15 μL) inhibits pruritus in mice induced by intradermal injection of mmu-miR-711 (1 mM, 10 μL) but has no effects on pruritus evoked by chloroquine (CQ, 100 μg/10 μL) and compound 48/80 (48/80, 50 μg/10 μL). **p<0.01, ***p<0.001, one-way ANOVA, n=5 mice/group. Data are represented as mean±SEM. See also FIG. 14A-FIG. 14C.

FIG. 7A-FIG. 7G. A Mouse Model of CTCL Showing Chronic Itch and miR-711 Upregulation. (FIG. 7A) Images of lymphomas on back skins at 15, 20, 25, 30, and 40 days after inoculation by intradermal injection of CD4⁺ Myla cells (1×10⁵ cells/μL, 100 μL). Scale, 10 mm. (FIG. 7B) Images of DAPI staining of normal and tumor-bearing skins after CTCL. Scale, 1 mm. (FIG. 7C) Time course of tumor growth, revealed by diameters of tumors after inoculation of CD4⁺ Myla cells. (FIG. 7D) Time course of CTCL-evoked chronic itch. Number of scratches in 1 hr was counted blindly from the recorded videos. \hat{ }\hat{ }p<0.01, \hat{ }\hat{ }\hat{ }p≤0.001, one-way ANOVA, versus baseline (BL), n=6-9 mice/group. (FIG. 7E) Relative serum levels of hsa-miR-21, hsa-miR-155, hsa-miR-326, and hsa-miR-711 of naive and CTCL mice. \hat{ }p<0.05, \hat{ }\hat{ }p<0.01, versus respective naïve mice, one-way ANOVA, n=3-4 mice/group. (FIG. 7F) In situ hybridization (red) showing hsa-miR-711 expression in lymphoma cells on the back skin 20 days after CTCL induction. The skin sections were counterstained with DAPI (blue) to label nuclei. Note no signal is detected by the control probe. Scale, 100 μm. (FIG. 7F′) Enlarged box in (FIG. 7F) showing single and double staining. Scale, 25 μm. (FIG. 7G) Quantification of hsa-miR-711-positive cells per square mm back skin at different times of CTCL. \hat{ }p<0.05, \hat{ }\hat{ }p<0.01, versus naive control, one-way ANOVA, n=4 mice/group. Data are represented as mean±SEM. See also FIG. 15A-FIG. 15B.

FIG. 8A-FIG. 8E. Inhibition of Chronic Itch by miR-711 Inhibitor, TRPA1 Antagonists, and miR-711/TRPA1 Interaction Blocking Peptide in a Mouse Model of CTCL. (FIG. 8A) Inhibition of CTCL-evoked chronic itch by intradermal injection of hsa-miR-711 inhibitor (100 μM with a complementary sequence to hsa-miR-711) and TRPA1 antagonists (200 μM HC030031 and 50 μM A967079), 20 days after CD4⁺ Myla cell inoculation. ***p<0.001, \hat{ }\hat{ }\hat{ }p<0.001, versus vehicle, one-way ANOVA, n=6 mice/group. (FIG. 8B) Inhibition of CTCL-evoked chronic itch by the blocking peptide (2 mM), given 20 days after the Myla cell inoculation. *p<0.05, ***p<0.001, ^{###}p<0.001, two-way ANOVA, n=6-7 mice/group. BL, baseline. (FIG. 8C) Overexpression of hsa-miR-711 inhibitor in Myla cells via lentivirus (LV) before the inoculation attenuates chronic itch after CTCL. *p<0.05, **p<0.01, ***p<0.001, versus Mock control, ^{###}p<0.001, two-way ANOVA, n=5-7 mice/group. (FIG. 8D) Overexpression of hsa-miR-711 via adenovirus (AV, 10 μL, titer of 2×10¹¹ GC/μL) induces persistent itch after intradermal AV injection on the back skin. ***p<0.001, versus control AV-GFP, ^{###}p<0.001, versus control AV-GFP, two-way ANOVA, n=6 mice/group. (FIG. 8E) Inhibition of hsa-miR-711 AV-induced persistent itch by intradermal injection of A-967079 (50 μM), hsa-miR-711 inhibitor (100 μM), and the blocking peptide (2 μM) 10 days after the AV injection. *p<0.05, **p<0.01, ***p<0.001, ^{##}p<0.01, ^{###}p<0.001, two-way ANOVA, n=5-6 mice/group. Data are represented as mean±SEM. See also FIG. 15A-FIG. 15B and FIG. 16A-FIG. 16G.

FIG. 9A-FIG. 9F. Characterization of miR-711-induced itch in the cheek model in mice. (FIG. 9A) Intradermal injection of miR-711 induces dose-dependent scratching, but intradermal AITC evokes both pain and itch. ***P<0.001, vs. vehicle, One-Way ANOVA, n=6 mice/group. ^{###}P<0.001 vs. vehicle, Two-tailed Student's t-test, n=6 mice/group. Note that miR-711 fails to induce wiping at all the concentrations. In contrast, intradermal AITC induces scratching at low concentrations but wiping at high concentrations. *P<0.05, ***P<0.001, vs. vehicle, One-Way ANOVA, n=7 mice/group. (FIG. 9B) Intradermal miRNA-711 at the highest concentration (5 mM) causes skin lesion on the cheek, as indicated by blue circle, 1 h after intradermal injection. (FIG. 9C) Analysis of acute itch within the first 60 sec following intradermal cheek injection of miR-711 (1 mM, 10 μL), chloroquine (CQ, 100 μg in 10 μL), and histamine (200 μg in 10 μL). Note a faster induction of scratching by miR-711 within first 10 sec after the injection. n=6 mice/group. (FIG. 9D) Sequence alignment of miR-711 in different species. Note that the GGGACCC core sequence is identical in all the species. (FIG. 9E, FIG. 9F) Intradermal miR-711 also evokes marked pruritus on the back of mice. (FIG. 9E) Intradermal nape injection of mmu-miR-711, but not mmu-miR-21, mmu-miR-155, or mmu-miR-326 at 1 mM (10 μL), induces scratching. ***P<0.001, vs. vehicle, One-Way ANOVA, n=6 mice/group. (FIG. 9F) Intradermal nape injection of miR-711 induces dose-dependent scratching. **P<0.01, ***P<0.001, vs. vehicle, One-Way ANOVA, n=6 mice/group. Data are Mean±SEM.

FIG. 10A-FIG. 10E. Additional characterization of TRPA1 activation by miR-711 and AITC in HEK293 cells expressing hTRPA1. (FIG. 10A, FIG. 10B) miR-711 (10 μM) does not cause TRPA1 desensitization after the 2^{nd} application. (FIG. 10A) Traces of inward currents. (FIG. 10B) Quantification of inward current induced by first and second application of miR-711 (10 μM). N.S., not significant, Two-tailed Student's t-test, n=10 cells/group. (FIG. 10C) Single channel conductance of hTRPA1 activated by mmu-miR-711 and AITC in HEK293 cells expressing hTRPA1. Related to outside-out recordings in FIG. 2H and FIG. 2I. N.S., not significant, Two-tailed Student's t-test, n=5 cells/group. (FIG. 10D) I/V analysis shows different permeability to calcium and sodium in Trpa1-expressing HEK293 cells in response to miR-711 (10 μM) and AITC (50 μM). (FIG. 10E) Quantification of reverse potential to Ca²⁺ and Na⁺, ***P<0.001, Two-tailed Student's t-test, n=5-10 cells per group. Note that AITC and miR-711 cause distinctive permeability changes in Ca²⁺ and Na⁺. Data are Mean±SEM.

FIG. 11A-FIG. 11H. miR-711 induces calcium responses in hTRPA1-expressing HEK293 cells and dissociated DRG and trigeminal ganglion (TG) neurons of Pirt-GCaMP3 mice. (FIG. 11A) Representative images of calcium changes in HEK293 cells in response to mmu-miR-711 (50 μM) and AITC (50 μM). Cells were incubated with 2 μM Fura-2 for 40 min. Scale is 50 μm. (FIG. 11B) Typical calcium traces show a HEK293 cell response to miR-711 and AITC. (FIG. 11C-FIG. 11E) miR-711 (50 μM) evoked calcium responses in mouse DRG neurons of Pirt-GCaMP3 mice before and after the treatment of TRPA1 antagonist A967079 (10 μM). (FIG. 11C) Representative images of DRG neurons. Scale is 50 μm. (FIG. 11D) Typical calcium trace of a mouse neuron. (FIG. 11E) Quantification of calcium response. Note 15 of 310 (4.8%) neurons showed calcium responses to miR-711, which is completely blocked by A967079. *P<0.001, t-test, n=15. (FIG. 11F-FIG. 11H) Calcium responses in TG neurons of Pirt-GCaMP3 mice. (FIG. 11F) Representative images of TG neurons in response to mmu-miR-711 (50 μM), histamine (His, 500 μM), chloroquine (CQ, 1000 μM), and AITC (200 μM). Scale is 50 μm. (FIG. 11G) Typical calcium traces of a TG neuron in response to mmu-miR-711, Histamine, CQ, and AITC. (FIG. 11H) Venn diagram showing overlaps between miR-711-responvie neurons and histamine, CQ, and AITC responsive neurons and the percentage of each population in TG neurons. A total of 204 neurons from 3 mice were analyzed, and 13 TG neurons respond to all the stimuli.

FIG. 12A-FIG. 12F. Action potentials, calcium currents, and resting membrane potentials of mouse DRG neurons and inward currents in human DRG neurons following miR-711 treatment. (FIG. 12A) The resting membrane potentials (RMPs) of DRG neurons prior to the treatment of miR-711 (10 μM) and AITC (50 μM). n=7-9 neurons per group. Notice all DRG neurons have similar RMPs before the treatment. (FIG. 12B-FIG. 12D) miR-711 (10 μM) does not inhibit calcium currents in dissociated small-diameter mouse DRG neurons. (FIG. 12B) Trace of calcium currents before and after mmu-miR-711 (10 μM) treatment. (FIG. 12C) Time-course of relative calcium currents. DRG neurons were treated with miR-711 (10 μM) for 1 min, n=6 neurons. (FIG. 12D) Quantification of calcium currents before and after miR-711 perfusion. N.S., not significant, Two-tailed Student's t-test, n=6 neurons. (FIG. 12E, FIG. 12F) Inward currents evoked by miRNAs and AITC in dissociated human DRG neurons with small diameters (<50 μm). (FIG. 12E) hsa-miR-711 and hsa-miR-642b (10 μM) evoke TRPA1-dependent inward currents in human DRG neurons. Note the currents were blocked by A967079 (10 μM). (FIG. 12F) Quantification of inward currents in human DRG neurons. *P<0.05, ***P<0.001, Two-tailed Student's t-test, n=4 neurons per group from 4 donors. Data are Mean±SEM.

FIG. 13A-FIG. 13K. Computer simulation shows the interactions between hTRPA1 and the core sequence of miR-711. (FIG. 13A) Cluster population of high affinity GGGACCC/TRPA1 conformations. The ensemble of high affinity GGGACCC/TRPA1 conformations (i.e., binding energy lower or equal to −75 kcal/mol) were clustered according to the RSMD computed over the GGGACCC phosphorus atoms, using a cutoff of 4.24 Å to distinguish two distinct conformations. The conformations of the most populated clusters (˜4% of the isolated conformational space) were used to explore the binding mode of GGGACCC to hTRPA1 and the lowest binding energy conformation of the ensemble (i.e., ˜87 kcal/mol) is chosen as the representative structure of miRNA-711-TRPA1 complex. (FIG. 13B) Fluctuation of TRPA1-bound GGGACCC conformation. The average RMSD (in black) and standard deviation (grey) of GGGACCC phosphorus atoms is computed over five independent, 4.5×10⁵ step-long DMD simulations at temperature 0.3 kcal/(mol k_{B}). (FIG. 13C) Fluctuations of inter-atomic distances between GGGACCC and TRPA1. Standard deviation of distances between atoms of GGGACCC and TRPA1 residues interacting within 5 Å of each nucleobase are computed over five independent, 4.5×10⁵ step-long DMD simulations at temperature 0.3 kcal/(mol k_{B}). (FIG. 13D-FIG. 13K) Detailed views of the binding interactions between each nucleotide of the GGGACCC core sequence and hTRPA1, including G001 (FIG. 13E), G002 (FIG. 13F), G003 (FIG. 13G), A004 (FIG. 13H), C005 (FIG. 13I), C006 (FIG. 13J), and C007 (FIG. 13K), with special focus on P937 with G003 and A004 within 5 Å. P937 was highlighted in red.

FIG. 14A-FIG. 14C. Alignment of TRPA1 sequences of different species and effects of mTRPA1 mutations on inward currents induced by ATIC and miR-711 in CHO cells. (FIG. 14A) Amino acids sequence alignment of human, mouse, and rat TRPA1. S1-S6 are six transmembrane segments indicated by blue lines. Predicated residues with possible interactions with miR-711 core sequence are shown with red lines. Ultra-conservative amino acids among all three species are highlighted in yellow. Blue box indicates the residue P934 of hTRPA1, which is equivalent to P937 of mTRPA1. (FIG. 14B, FIG. 14. C) Effects of mTRPA1 mutations on AITC- or miR-711-induced inward current in CHO cells transfected with wild type or mutant Trpa1 cDNAs. (FIG. 14B) Schematic of mTRPA1 domains on cell membrane. Predicted and ultra-conservative amino acids were highlighted in red, predicted but non-conservative amino acids were labeled in purple, and randomly selected and non-predicted amino acids were labeled in green. Black residues are non-mutated ones. A total of 13 mutants in extracellular loop 1-3 were generated as indicated. (FIG. 14C) Summary of different mutants of mTRPA1 and their effects (% inhibition) on AITC (50 μM) or miR-711 (10 μM) induced inward currents in CHO cells transfected with wild type or mutated Trpa1 cDNAs. Red residues are conservative ones predicted by computer simulation, and green residues were randomly selected on extracellular loops. Purple residues are non-conservative sites predicted by computer simulation. Specific mutations (M11 and M13) that only cause reduction in miR-711 but not AITC currents (bold in black) are underlined. n=6-15 cells per condition.

FIG. 15A-FIG. 15B. Characterization of lymphomas on the back skin of CTCL mice. (FIG. 15A) Images of DAPI staining of normal and tumor-bearing skins after CTCL. Scale, 1 mm. (FIG. 15A′) Enlarged box-1 and box-2 in FIG. 15A. Scale, 250 μm. (FIG. 15B) Images of HE staining of normal and tumor-bearing skins after CTCL. Scale, 500 μm. Dashed lines indicate the epidermis.

FIG. 16A-FIG. 16G. Characterization of miR-711 secretion in culture media and mouse serum and nerve innervation and tumor growth in the CTCL model. (FIG. 16A) hsa-miR-711 secretion in tumor cell cultures. A total of 1 million B16 cells, HuT102 cells, and CD4⁺ Myla cells were plated into 2 mL culture medium, and 0.2 mL of medium were collected 24 h after subculture for qRT-PCR analysis. Note that melanoma B16 cells do not secrete hsa-miR-711.***P<0.001, vs. B16, One-Way ANOVA, n=3 replications/group. (FIG. 16B) Time course of hsa-miR-711 secretion in culture media. One million CD4⁺ Myla cells were plated into 2 mL culture medium, 0.2 mL medium was collected at 0 h, 12 h, and 24 h after subculture for qRT-PCR analysis. **P<0.001, vs. 0 h, One-Way ANOVA, n=3 replications/group. (FIG. 16C, FIG. 16D) Secretion of hsa-miR-711 and mmu-miR-711 in serum of control and CTCL mice. (FIG. 16C) Copy number of hsa-miR-711 and mmu-miR-711 in serum of naïve, CTCL 20 d, and CTCL 40 d mice. (FIG. 16D) Ct values of miRNA levels shown in FIG. 16C. (FIG. 16E) Nerve innervation, as revealed by PGP 9.5 immunostaining, in the skin lymphoma 20 days after Myla cell inoculation. DAPI staining shows all the nuclei of the tumor cells. Scale, 100 μm. (FIG. 16F, FIG. 16G) Tumor growth and miR-711 secretion after lentivirus (LV) over-expression of miR-711 inhibitor in Myla cells before the inoculation. (FIG. 16F) miR-711 inhibitor does not affect the tumor growth. Two-Way ANOVA, n=7 mice/group. (FIG. 16G) Relative serum expression levels of miR-711 10 days after nape injection of AV-GFP (Mock control) and AV-miR-711-GFP (10 μL, titer of 2×10¹¹ GC/mL). ***P<0.001 vs. AV-GFP control, Two-tailed Student's t test, n=3-4 mice/group. Data are Mean±SEM.

### DETAILED DESCRIPTION

Described herein are compositions for inhibiting miR-711 and for inhibiting the interaction of miR-711 with TRPA1. As detailed herein, intradermal cheek injection of miR-711 induces TRPA1-depedent itch (scratching) without pain (wiping) in naive mice. Extracellular perfusion of miR-711 induces TRPA1 currents in both Trpa1-expressing heterologous cells and native sensory neurons through the core sequence GGGACCC (SEQ ID NO: 1). The core sequence binds several residues at the extracellular S5-S6 loop of TRPA1. Lymphoma-induced chronic itch may be suppressed by miR-711 inhibition and a blocking peptide that disrupts the miR-711/TRPA1 interaction. The compositions detailed herein may be used to treat a disease or condition such as pruritis.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4. In some embodiments, the term “about” as used herein as applied to one or more values of interest, refers to a value that is similar to a stated reference value. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The term “antagonist” or “inhibitor” refers to a molecule which blocks (e.g., reduces or prevents) a biological activity.

As used herein, the term “agonist” refers to a molecule or compound that triggers (e.g., initiates or promotes), partially or fully enhances, stimulates, or activates one or more biological activities. An agonist may mimic the action of a naturally occurring substance. Whereas an agonist causes an action, an antagonist blocks the action of the agonist.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, Tex.; SAS Institute Inc., Cary, N.C.). The healthy or normal levels or ranges for a target or for an activity may be defined in accordance with standard practice. A control may be a subject without a miR-711 inhibitor as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

A “pharmaceutically acceptable excipient,” “pharmaceutically acceptable diluent,” “pharmaceutically acceptable carrier,” or “pharmaceutically acceptable adjuvant” as used interchangeably herein means an excipient, diluent, carrier, and/or adjuvant that is useful in preparing a pharmaceutical composition that is generally safe, non-toxic, and neither biologically nor otherwise undesirable, and includes an excipient, diluent, carrier, and adjuvant that is acceptable for veterinary use and/or human pharmaceutical use, such as those promulgated by the United States Food and Drug Administration.

“Polynucleotide” as used herein can be single stranded or double stranded, or can contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including uracil, adenine, thymine, cytosine, guanine, inosine, xanthine hypoxanthine, isocytosine, and isoguanine. Polynucleotides can be obtained by chemical synthesis methods or by recombinant methods.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three dimensional structure of a polypeptide monomer. “Quaternary structure” refers to the three dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Sample” or “test sample” as used herein can mean any sample in which the presence and/or level of a target is to be detected or determined or any sample comprising a miR-711 inhibitor as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

The term “specificity” as used herein refers to the number of true negatives divided by the number of true negatives plus the number of false positives, where specificity (“spec”) may be within the range of 0<spec<1. Ideally, the methods described herein have the number of false positives equaling zero or close to equaling zero, so that no subject is wrongly identified as having a disease when they do not in fact have disease. Hence, a method that has both sensitivity and specificity equaling one, or 100%, is preferred.

By “specifically binds,” it is generally meant that an agent or polynucleotide or polypeptide binds to a target when it binds to that target more readily than it would bind to a random, unrelated target.

“Subject” as used herein can mean a mammal that wants or is in need of the herein described miR-711 inhibitors or methods. The subject may be a patient. The subject may be a human or a non-human animal. The subject may be a mammal. The mammal may be a primate or a non-primate. The mammal can be a primate such as a human; a non-primate such as, for example, dog, cat, horse, cow, pig, mouse, rat, camel, llama, goat, rabbit, sheep, hamster, and guinea pig; or non-human primate such as, for example, monkey, chimpanzee, gorilla, orangutan, and gibbon. The subject may be male. The subject may be female. In some embodiments, the subject is human. The subject may be of any age or stage of development, such as, for example, an adult, an adolescent, or an infant. In some embodiments, the subject has a specific genetic marker. The subject may be male or female. The subject may be diagnosed with or at risk of developing disease. The subject or patient may be undergoing other forms of treatment.

“Substantially identical” can mean that a first and second amino acid sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids.

“Treat,” “treatment,” or “treating,” when referring to protection of a subject from a disease, means suppressing, repressing, ameliorating, or completely eliminating the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease.

“Variant” as used herein with respect to a polynucleotide means (i) a portion or fragment of a referenced nucleotide sequence; (ii) the complement of a referenced nucleotide sequence or portion thereof; (iii) a polynucleotide that is substantially identical to a referenced polynucleotide or the complement thereof; or (iv) a polynucleotide that hybridizes under stringent conditions to the referenced polynucleotide, complement thereof, or a sequences substantially identical thereto.

A “variant” can further be defined as a peptide or polypeptide that differs in amino acid sequence by the insertion, deletion, or conservative substitution of amino acids, but retain at least one biological activity. Representative examples of “biological activity” include the ability to be bound by a specific antibody or polypeptide or to promote an immune response. Variant can mean a substantially identical sequence. Variant can mean a functional fragment thereof. Variant can also mean multiple copies of a polypeptide. The multiple copies can be in tandem or separated by a linker. Variant can also mean a polypeptide with an amino acid sequence that is substantially identical to a referenced polypeptide with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids. See Kyte et al., J. Mol. Biol. 1982, 157, 105-132. The hydropathic index of an amino acid is based on a consideration of its hydrophobicity and charge. It is known in the art that amino acids of similar hydropathic indexes can be substituted and still retain protein function. In one aspect, amino acids having hydropathic indices of ±2 are substituted. The hydrophobicity of amino acids can also be used to reveal substitutions that would result in polypeptides retaining biological function. A consideration of the hydrophilicity of amino acids in the context of a polypeptide permits calculation of the greatest local average hydrophilicity of that polypeptide, a useful measure that has been reported to correlate well with antigenicity and immunogenicity, as discussed in U.S. Pat. No. 4,554,101, which is fully incorporated herein by reference. Substitution of amino acids having similar hydrophilicity values can result in polypeptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant can be a polynucleotide sequence that is substantially identical over the full length of the full gene sequence or a fragment thereof. The polynucleotide sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant can be an amino acid sequence that is substantially identical over the full length of the amino acid sequence or fragment thereof. The amino acid sequence can be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

### 2. MIR-711

MicroRNAs (miRNAs) bind the 3′ untranslated regions of mRNAs to regulate gene expression post-transcription. miR-711 is a miRNA having a core polynucleotide sequence of SEQ ID NO: 1 (GGGACCC). In some embodiments, miR-711 comprises a polynucleotide sequence of SEQ ID NO: 1. In some embodiments, miR-711 comprises a polynucleotide sequence of SEQ ID NO: 2. miR-711 may bind to TRPA1. miR-711 may bind to the extracellular side of TRPA1. miR-711 may bind to TRPA1 at S5-S6 loop. miR-711 may bind to TRPA1 at P934 (of human TRPA1).

a. miR-711 Inhibitor

Provided herein are miR-711 inhibitors. A miR-711 inhibitor may comprise a biological molecule, including nucleic acid molecules, such as a polynucleotide having RNAi activity against miR-711 or a fragment or substrate thereof. In some embodiments, the nucleic acid molecules include RNAs, dsRNAs, miRNAs, siRNAs, nucleic acid aptamers, antisense nucleic acid molecules, and enzymatic nucleic acid molecules that comprise a sequence that is sufficient to allow for binding to an encoding nucleic acid sequence and inhibit activity thereof (i.e., are complementary to such encoding nucleic acid sequences). Suitably, an RNAi molecule comprises a sequence that is complementary to at least a portion of a target sequence such that the RNAi can hybridize to the target sequence under physiological or artificially defined (e.g., reaction) conditions. In some embodiments an RNAi molecule comprises a sequence that is complementary such that the molecule can hybridize to a target sequence under moderate or high stringency conditions, which are well known and can be determined by one of skill in the art. In some embodiments an RNAi molecule has complete (100%) complementarity over its entire length to a target sequence. A variety of RNAi molecules are known in the art, and can include chemical modifications, such as modifications to the sugar-phosphate backbone or nucleobase that are known in the art. The modifications may be selected by one of skill in the art to alter activity, binding, immune response, or other properties. In some embodiments, the RNAi can comprise an siRNA having a length from about 18 to about 24 nucleotides, about 5 to about 50 nucleotides, about 5 to about 30 nucleotides, or about 10 to about 20 nucleotides.

In some embodiments, the inhibitory nucleic acid molecule can bind to a target nucleic acid sequence under stringent binding conditions. The terms “stringent conditions” or “stringent hybridization conditions” includes reference to conditions under which a polynucleotide will hybridize to its target sequence, to a detectably greater degree than other sequences (e.g., at least 2-fold over background). An example of stringent conditions include those in which hybridization in 50% formamide, 1 M NaCl, 1% SDS at 37° C., and a wash in 0.1×SSC at 60 to 65° C. is performed. Amino acid and polynucleotide identity, homology and/or similarity can be determined using the ClustalW algorithm, MEGALIGN™ (Lasergene, Wis.). Given a target polynucleotide sequence, for example of miR-711 or biological substrate thereof, an inhibitory nucleic acid molecule can be designed using motifs and targeted to a region that is anticipated to be effective for inhibitory activity, such as is known in the art.

miR-711 inhibitors may include, for example, a miR-711/TRPA1 interaction blocking peptide, or a polynucleotide complementary to miR-711 or to a portion or fragment thereof. In some embodiments, the miR-711/TRPA1 interaction blocking peptide comprises a polypeptide having an amino acid sequence of SEQ ID NO: 3 or SEQ ID NO: 4 or a variant or fragment or portion thereof.

The miR-711 inhibitor may decrease the amount of, or the biological activity of miR-711. The miR-711 inhibitor may elicit a variety of effects such as for example, inhibiting nerve fibers expressing TRPA1, inhibiting the binding of miR-711 to TRPA1, inhibiting the binding of miR-711 to the extracellular side of TRPA1, inhibiting the binding of miR-711 to TRPA1 at S5-S6 loop, inhibiting binding of miR-711 to TRPA1 at P934 (of hTRPA1), neutralizing extracellular miR-711, or a combination thereof. In some embodiments, the miR-711 inhibitor may also be referred to as a TRPA1 inhibitor. The miR-711 inhibitor may inhibit an activity or expression of miR-711 by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, or at least about 50-fold.

### 3. TRPA1

Transient receptor potential cation channel, subfamily A, member 1 (TRPA1) is also known as transient receptor potential ankyrin 1. TRPA1 is an ion channel located on the plasma membrane of many human and animal cells. The TRPA1 ion channel may be a sensor for environmental irritants giving rise to somatosensory modalities such as pain, cold, and itch. Primary sensory neurons, especially nociceptors, express TRPA1 for pain sensation and sensitization. TRPA1 is also expressed by pruriceptive neurons (pruriceptors) and regulates acute and chronic itch as well as pain. TRPA1 may comprise a polypeptide having an amino acid sequence of SEQ ID NO: 55, SEQ ID NO: 56, or SEQ ID NO: 57, for example, or a variant or fragment or portion thereof. In some embodiments, TRPA1 comprises a polypeptide having an amino acid sequence of SEQ ID NO: 55. In some embodiments, TRPA1 comprises a polypeptide having an amino acid sequence of SEQ ID NO: 56. In some embodiments, TRPA1 comprises a polypeptide having an amino acid sequence of SEQ ID NO: 57.

a. TRPA1 Inhibitor

Further provided herein are TRPA1 inhibitors. In some embodiments, a TRPA1 inhibitor is administered in addition to the miR-711 inhibitor. The TRPA1 inhibitor may be administered before the miR-711 inhibitor, after the miR-711 inhibitor, or co-administered with the miR-711 inhibitor, or a combination thereof. The TRPA1 inhibitor can inhibit the biological function of TRPA1 (e.g., inhibit cation channel activity, inhibit Ca++ transport and/or availability). Other embodiments provide for a TRPA1 inhibitor that may inhibit the expression of mRNA encoding TRPA1. Some embodiments provide a TRPA1 inhibitor that may inhibit the translation of mRNA encoding TRPA1 to protein. Thus, a TRPA1 inhibitor may indirectly or directly bind and inhibit the activity of TRPA1 (e.g., binding activity or enzymatic activity), reduce the expression of TRPA1, prevent expression of TRPA1, or inhibit the production of TRPA1 in a cell. Inhibit or inhibiting relates to any measurable reduction or attenuation of amounts or activity, e.g., amounts or activity of TRPA1, such as those disclosed herein. “Amounts” and “levels” of protein or expression may be used herein interchangeably.

In some embodiments, a TRPA1 inhibitor can increase the amount of, or the biological activity of, a protein that can reduce the activity of TRPA1. Inhibitors capable of increasing the level of such a protein may include any inhibitor capable of increasing protein or mRNA levels or increasing the expression of the protein that inhibits TRPA1. In one embodiment, a TRPA1 inhibitor may comprise the protein itself. For example, a TRPA1 inhibitor may include exogenously expressed and isolated protein capable of being delivered to the cells. The protein may be delivered to cells by a variety of methods, including fusion to Tat or VP16 or via a delivery vehicle, such as a liposome, all of which allow delivery of protein-based inhibitors across the cellular membrane. Those of skill in the art will appreciate that other delivery mechanisms for proteins may be used. Alternatively, mRNA expression of the TRPA1 inhibitor may be enhanced relative to control cells by contact with a TRPA1 inhibitor. For example, an inhibitor capable of increasing the level of a natively expressed protein that inhibits TRPA1 may include a gene expression activator or de-repressor. As another example, a TRPA1 inhibitor capable of decreasing the level of natively expressed TRPA1 protein may include a gene expression repressor. An inhibitor capable of increasing the level of a protein that inhibits TRPA1 may also include inhibitors that bind to directly or indirectly and increase the effective level of the protein, for example, by enhancing the binding or other activity of the protein. An inhibitor capable of decreasing the level of TRPA1 protein may also include compounds or compositions that bind to directly or indirectly and decrease the effective level of TRPA1 protein, for example, by inhibiting or reducing the binding or other activity of the TRPA1 protein.

The amount or level of expression of a biomolecule (e.g., mRNA or protein) in a cell may be evaluated by any variety of techniques that are known in the art. For example, the inhibition of the level of protein expression (e.g., TRPA1) may be evaluated at the protein or mRNA level using techniques including, but not limited to, Western blot, ELISA, Northern blot, real time PCR, immunofluorescence, or FACS analysis. For example, the expression level of a protein may be evaluated by immunofluorescence by visualizing cells stained with a fluorescently-labeled protein-specific antibody, Western blot analysis of protein expression, and RT-PCR of protein transcripts. The expression level of TRPA1 may be compared to a control. The comparison may be made to the level of expression in a control cell, such as a non-disease cell or other normal cell. Alternatively the control may include an average range of the level of expression from a population of normal cells. Alternatively, a standard value developed by analyzing the results of a population of cells with known responses to therapies or agents may be used. Those skilled in the art will appreciate that any of a variety of controls may be used.

A TRPA1 inhibitor may include one or more compounds and compositions. In some embodiments, a TRPA1 inhibitor comprises a compound. In some embodiments, a TRPA1 inhibitor is a compound. In some embodiments, a TRPA1 inhibitor comprises a small molecule. In some embodiments, a TRPA1 inhibitor is a small molecule. A TRPA1 inhibitor may comprise a biological molecule, including nucleic acid molecules, such as a polynucleotide having RNAi activity against TRPA1 or a substrate thereof. In some embodiments, the nucleic acid molecules include RNAs, dsRNAs, miRNAs, siRNAs, nucleic acid aptamers, antisense nucleic acid molecules, and enzymatic nucleic acid molecules that comprise a sequence that is sufficient to allow for binding to an encoding nucleic acid sequence and inhibit activity thereof (i.e., are complementary to such encoding nucleic acid sequences). Suitably, an RNAi molecule comprises a sequence that is complementary to at least a portion of a target sequence such that the RNAi can hybridize to the target sequence under physiological or artificially defined (e.g., reaction) conditions. In some embodiments an RNAi molecule comprises a sequence that is complementary such that the molecule can hybridize to a target sequence under moderate or high stringency conditions, which are well known and can be determined by one of skill in the art. In some embodiments an RNAi molecule has complete (100%) complementarity over its entire length to a target sequence. A variety of RNAi molecules are known in the art, and can include chemical modifications, such as modifications to the sugar-phosphate backbone or nucleobase that are known in the art. The modifications may be selected by one of skill in the art to alter activity, binding, immune response, or other properties. In some embodiments, the RNAi can comprise an siRNA having a length from about 18 to about 24 nucleotides, about 5 to about 50 nucleotides, about 5 to about 30 nucleotides, or about 10 to about 20 nucleotides.

In some embodiments, the inhibitory nucleic acid molecule can bind to a target nucleic acid sequence under stringent binding conditions. The terms “stringent conditions” or “stringent hybridization conditions” includes reference to conditions under which a polynucleotide will hybridize to its target sequence, to a detectably greater degree than other sequences (e.g., at least 2-fold over background). An example of stringent conditions include those in which hybridization in 50% formamide, 1 M NaCl, 1% SDS at 37° C., and a wash in 0.1×SSC at 60 to 65° C. is performed. Amino acid and polynucleotide identity, homology and/or similarity can be determined using the ClustalW algorithm, MEGALIGN™ (Lasergene, Wis.). Given a target polynucleotide sequence, for example of TRPA1 or biological substrate thereof, an inhibitory nucleic acid molecule can be designed using motifs and targeted to a region that is anticipated to be effective for inhibitory activity, such as is known in the art.

In other embodiments, a TRPA1 inhibitor comprises an antibody that can specifically bind to a protein such as TRPA1 or a fragment thereof. Embodiments also provide for an antibody that inhibits TRPA1 through specific binding to TRPA1. The antibodies can be produced by any method known in the art, such as by immunization with a full-length protein such as TRPA1, or fragments thereof. The antibodies can be polyclonal or monoclonal, and/or may be recombinant antibodies. In embodiments, antibodies that are human antibodies can be prepared, for example, by immunization of transgenic animals capable of producing a human antibody (see, for example, International Patent Application Publication No. WO 93/12227). Monoclonal antibodies (mAbs) can be produced by a variety of techniques, including conventional monoclonal antibody methodology, e.g., the standard somatic cell hybridization technique of Kohler and Milstein, and other techniques, e.g., viral or oncogenic transformation of B-lymphocytes. Animal systems for preparing hybridomas include mouse. Hybridoma production in the mouse is very well established, and immunization protocols and techniques for isolation of immunized splenocytes for fusion are well known in the art. Fusion partners (e.g., murine myeloma cells) and fusion procedures are also known.

Any suitable methods can be used to evaluate a candidate active compound or composition for inhibitory activity toward TRPA1. Such methods can include, for example, in vitro assays, in vitro cell-based assays, ex vivo assays, and in vivo methods. The methods can evaluate binding activity, or an activity downstream of the enzyme of interest. Ex vivo assays may involve treatment of cells with an inhibitor of the invention, followed by detection of changes in transcription levels of certain genes, such as TRPA1 through collection of cellular RNA, conversion to cDNA, and quantification by quantitative real time polymerase chain reaction (RT-QPCR). Additionally, the cell viability or inflammation may be determined after treatment with an inhibitor.

TRPA1 inhibitors may include, for example, HC030031 and A967079. TRPA1 inhibitors may include any other TRPA1 inhibitors known in the art. HC030031 (2-(1,3-Dimethyl-2,6-dioxo-1,2,3,6-tetrahydro-7H-purin-7-yl)-N-(4-isopropylphenyl)acetamide) may comprise a compound according to the below, or a pharmaceutically acceptable salt thereof:

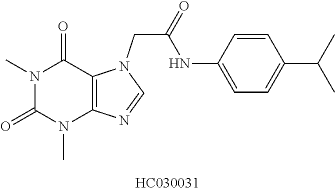 [SMILES: CC(C)c1ccc(NC(=O)Cn2cnc3c2c(=O)n(C)c(=O)n3C)cc1]

A967079 ((1E,3E)-1-(4-Fluorophenyl)-2-methyl-1-pentene-3-one oxime) may comprise a compound according to the below, or a pharmaceutically acceptable salt thereof:

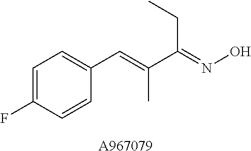

HC030031 and A967079 are commercially available. For example, HC030031 and A967079 are commercially available from Tocris Bioscience (Bristol, UK). Alternatively, HC030031 and A967079 may be synthetically made by methods known to one of skill in the art. The compound structure may be confirmed by methods known to one of skill in the art, such as, for example, mass spectrometry and NMR The compounds.

The present disclosure also includes an isotopically-labeled TRPA1 inhibitor, which is identical to a TRPA1 inhibitor compound shown above, for example, but for the fact that one or more atoms are replaced by an atom having an atomic mass or mass number different from the atomic mass or mass number usually found in nature. Examples of isotopes suitable for inclusion in the compounds of the invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³⁶Cl, respectively. Substitution with heavier isotopes such as deuterium, i.e. ²H, can afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated in the compound are ¹¹C, ¹³N, ¹⁵O, and ¹⁸F. Isotopically-labeled compounds can generally be prepared by conventional techniques known to those skilled in the art using appropriate isotopically-labeled reagent in place of non-isotopically-labeled reagent.

The TRPA1 inhibitor compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric, and the like. The amino groups of the compounds may also be quaternized with alkyl chlorides, bromides and iodides such as methyl, ethyl, propyl, isopropyl, butyl, lauryl, myristyl, stearyl, and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

### 4. PRURITIS

The compositions and methods detailed herein, such as those including a miR-711 inhibitor, may be used to treat a disease or condition such as, for example, pruritis, atopic eczema, pruritis associated with lymphoma, pruritis associated with liver disease, and psoriasis. The liver disease may be chronic liver disease. Pruritis may also be referred to as itch. Pruritis is a sensation that causes the desire or reflex to scratch. While pain may evoke a withdrawal reflex, which leads to retraction and therefore a reaction trying to protect an endangered part of the body, itch in contrast may create a scratch reflex, which draws one to the affected skin site. The pruritis may be chronic or acute, or a combination thereof.

### 5. PHARMACEUTICAL COMPOSITIONS

The miR-711 inhibitors as detailed herein may be formulated into pharmaceutical compositions in accordance with standard techniques well known to those skilled in the pharmaceutical art. The composition may comprise the miR-711 inhibitor and a pharmaceutically acceptable carrier. The TRPA1 inhibitors as detailed herein may be formulated into pharmaceutical compositions in accordance with standard techniques well known to those skilled in the pharmaceutical art. The composition may comprise the TRPA1 inhibitor and a pharmaceutically acceptable carrier. In some embodiments, the miR-711 inhibitor and the TRPA1 inhibitor are co-administered in separate compositions. In some embodiments, the miR-711 inhibitor and the TRPA1 inhibitor are co-administered in the same composition. In such embodiments, the composition may comprise the miR-711 inhibitor and the TRPA1 inhibitor and a pharmaceutically acceptable carrier. The term “pharmaceutically acceptable carrier,” as used herein, means a non-toxic, inert solid, semi-solid or liquid filler, diluent, encapsulating material or formulation auxiliary of any type.

The route by which the disclosed miR-711 inhibitors are administered and the form of the composition will dictate the type of carrier to be used. The pharmaceutical composition may be in a variety of forms, suitable, for example, for systemic administration (e.g., oral, rectal, sublingual, buccal, implants, intranasal, intravaginal, transdermal, intravenous, intraarterial, intratumoral, intraperitoneal, or parenteral) or topical administration (e.g., dermal, pulmonary, nasal, aural, ocular, liposome delivery systems, or iontophoresis). In some embodiments, the pharmaceutical composition is for administration to a subject's central nervous system. In some embodiments, the pharmaceutical composition is for administration to a subject's skin. In some embodiments, the pharmaceutical composition is for topical administration. In some embodiments, the pharmaceutical composition is for intradermal injection. Techniques and formulations may generally be found in “Remington's Pharmaceutical Sciences,” (Meade Publishing Co., Easton, Pa.). Pharmaceutical compositions must typically be sterile and stable under the conditions of manufacture and storage. All carriers are optional in the compositions.

Pharmaceutically acceptable carriers include, for example, diluents, lubricants, binders, disintegrants, colorants, flavors, sweeteners, antioxidants, preservatives, glidants, solvents, suspending agents, wetting agents, surfactants, emollients, propellants, humectants, powders, pH adjusting agents, and combinations thereof. The pharmaceutical composition may include one or more adjuvants as known in the art.

Suitable diluents include, for example, sugars such as glucose, lactose, dextrose, and sucrose; diols such as propylene glycol; calcium carbonate; sodium carbonate; sugar alcohols, such as glycerin; mannitol; sorbitol; cellulose; starch; and gelatin. The amount of diluent(s) in a systemic or topical composition may typically be about 50 to about 90%.

Suitable lubricants include, for example, silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil, and oil of theobroma. The amount of lubricant(s) in a systemic or topical composition may typically be about 5 to about 10%.

Suitable binders include, for example, polyvinyl pyrrolidone; magnesium aluminum silicate; starches such as corn starch and potato starch; gelatin; tragacanth; sucrose; and cellulose and its derivatives, such as sodium carboxymethylcellulose, ethyl cellulose, methylcellulose, microcrystalline cellulose, and hydroxypropyl methylcellulose. The amount of binder(s) in a systemic composition may typically be about 5 to about 50%.

Suitable disintegrants include, for example, agar, alginic acid and the sodium salt thereof, effervescent mixtures, croscarmelose, crospovidone, sodium carboxymethyl starch, sodium starch glycolate, clays, and ion exchange resins. The amount of disintegrant(s) in a systemic or topical composition may typically be about 0.1 to about 10%.

Suitable preservatives include, for example, benzalkonium chloride, methyl paraben, and sodium benzoate. The amount of preservative(s) in a systemic or topical composition may typically be about 0.01 to about 5%.

Suitable glidants include, for example, silicon dioxide. The amount of glidant(s) in a systemic or topical composition may typically be about 1 to about 5%.

Suitable solvents include, for example, water, isotonic saline, ethyl oleate, glycerine, castor oils, hydroxylated castor oils, alcohols such as ethanol or isopropanol, methylene chloride, ethylene glycol monoethyl ether, diethylene glycol monobutyl ether, diethylene glycol monoethyl ether, dimethylsulfoxide, dimethyl formamide, tetrahydrofuran, and phosphate buffer solutions, and combinations thereof. The amount of solvent(s) in a systemic or topical composition is typically from about 0 to about 100%, or 0% to about 95%.

Suitable suspending agents include, for example, AVICEL RC-591 (from FMC Corporation of Philadelphia, Pa.) and sodium alginate. The amount of suspending agent(s) in a systemic or topical composition may typically be about 1 to about 8%.

Suitable surfactants include, for example, lecithin, Polysorbate 80, and sodium lauryl sulfate, and the TWEENS from Atlas Powder Company of Wilmington, Del. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition may typically be about 0.1% to about 5%.

Suitable emollients include, for example, stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition may typically be about 5% to about 95%.

Suitable propellants include, for example, propane, butane, isobutane, dimethyl ether, carbon dioxide, nitrous oxide, and combinations thereof. The amount of propellant in a topical composition may be about 0% to about 95%.

Suitable humectants include, for example, glycerin, sorbitol, sodium 2-pyrrolidone-5-carboxylate, soluble collagen, dibutyl phthalate, gelatin, and combinations thereof. The amount of humectant in a topical composition may be about 0% to about 95%.

Suitable powders include, for example, beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified Montmorillonite clay, hydrated aluminum silicate, fumed silica, carboxyvinyl polymer, sodium carboxymethyl cellulose, ethylene glycol monostearate, and combinations thereof. The amount of powder(s) in a topical composition may typically be 0% to 95%.

Suitable pH adjusting additives include, for example, HCl or NaOH in amounts sufficient to adjust the pH of a topical pharmaceutical composition.

Although the amounts of components in the compositions may vary depending on the type of composition prepared, in general, systemic compositions may include 0.01% to 50% of a miR-711 inhibitor and 50% to 99.99% of one or more carriers. Compositions for parenteral administration may typically include 0.1% to 10% of a compound and 90% to 99.9% of one or more carriers. Oral dosage forms may include, for example, at least about 5%, or about 25% to about 50% of a compound. The oral dosage compositions may include about 50% to about 95% of carriers, or from about 50% to about 75% of carriers. The amount of the carrier employed in conjunction with a disclosed compound is sufficient to provide a practical quantity of composition for administration per unit dose of the compound. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

### 6. ADMINISTRATION

“Administration” or “administering” refers to delivery of a compound or composition by any appropriate route to achieve the desired effect. The miR-711 inhibitors as detailed herein, or the pharmaceutical compositions comprising the same, may be administered to a subject or patient. Such compositions comprising a miR-711 inhibitor can be administered in dosages and by techniques well known to those skilled in the medical arts taking into consideration such factors as the age, sex, weight, and condition of the particular subject, and the route of administration.

The miR-711 inhibitor can be administered prophylactically or therapeutically. In prophylactic administration, the miR-711 inhibitor can be administered in an amount sufficient to induce a response. In therapeutic applications, the miR-711 inhibitors are administered to a subject in need thereof in an amount sufficient to elicit a therapeutic effect. An amount adequate to accomplish this is defined as “therapeutically effective amount.” Amounts effective for this use will depend on, e.g., the particular composition of the miR-711 inhibitor regimen administered, the manner of administration, the stage and severity of the disease, the general state of health of the patient, and the judgment of the prescribing physician. A therapeutically effective amount is also one in which any toxic or detrimental effects of a miR-711 inhibitor are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

For example, a therapeutically effective amount of a miR-711 inhibitor may be about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg. A therapeutically effective amount of a miR-711 inhibitor may be about 1×10⁶ to about 1×10¹⁰ cells per subject or dose. A therapeutically effective amount of a miR-711 inhibitor may be at least about 0.005 mM, at least about 0.006 mM, at least about 0.007 mM, at least about 0.008 mM, at least about 0.009 mM, at least about 0.01 mM, at least about 0.1 mM, at least about 0.2 mM, at least about 0.3 mM, at least about 0.4 mM, at least about 0.5 mM, at least about 0.6 mM, at least about 0.7 mM, at least about 0.8 mM, at least about 0.9 mM, at least about 1 mM, less than about 2 mM, less than about 1.5 mM, less than about 1.4 mM, less than about 1.3 mM, less than about 1.2 mM, less than about 1.1 mM, less than about 1 mM, less than about 0.9 mM, less than about 0.8 mM, less than about 0.7 mM, less than about 0.6 mM, less than about 0.5 mM, less than about 0.4 mM, less than about 0.3 mM, less than about 0.2 mM, about 0.005 mM to about 2 mM, or about 0.01 mM to about 1 mM.

The miR-711 inhibitor can be administered by methods well known in the art as described in Donnelly et al. (Ann. Rev. Immunol. 1997, 15, 617-648); Feigner et al. (U.S. Pat. No. 5,580,859, issued Dec. 3, 1996); Feigner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. The miR-711 inhibitor can be complexed to particles or beads that can be administered to an individual, for example, using a vaccine gun. One skilled in the art would know that the choice of a pharmaceutically acceptable carrier, including a physiologically acceptable compound, depends, for example, on the route of administration.

The miR-711 inhibitor can be delivered via a variety of routes. Typical delivery routes include parenteral administration, e.g., intradermal, intramuscular or subcutaneous delivery. Other routes include oral administration, intranasal, intravaginal, transdermal, intravenous, intraarterial, intratumoral, intraperitoneal, and epidermal routes. In some embodiments, the miR-711 inhibitor is administered intravenously, intraarterially, or intraperitoneally to the subject. In some embodiments, the miR-711 inhibitor is administered topically. In some embodiments, the miR-711 inhibitor is administered intradermally. In some embodiments, the miR-711 inhibitor is administered to the central nervous system of the subject. In some embodiments, the miR-711 inhibitor is administered to the subject intravenously.

The miR-711 inhibitor may be administered to a patient in a single dose or in multiple doses. In some embodiments, the miR-711 inhibitor is administered to the patient bi-weekly.

In embodiments including a TRPA1 inhibitor, the TRPA1 inhibitor may be administered as detailed above for the miR-711 inhibitor. The TRPA1 inhibitor may be administered before the miR-711 inhibitor, after the miR-711 inhibitor, or co-administered with the miR-711 inhibitor, or a combination thereof. As used herein the term “concomitant administration” or “co-administration” means that two compositions are administered to the same subject at the same time (simultaneously) or at about the same time, or that a single composition comprising both miR-711 inhibitor and TRPA1 inhibitor is administered to a subject. “At about the same time” encompasses sequential administration where the period between administrations is due only to the speed of the individual administering the active agents, rather than an intentional period of delay between administrations, e.g., the time period necessary for a single health care practitioner to administer a first composition according to accepted clinical practices and standards, and then administer a second composition according to accepted clinical practices and standards. In some embodiments, “at about the same time” encompasses administrations within a time period of fifteen minutes or less, thirty minutes or less, one hour or less, two hours or less, six hours or less, up to about twelve hours or less. Thus concomitant administration may occur in a time period of no more than about thirty minutes, or no more than about one hour, or no more than about two hours, and may not extend beyond 12 hours.

### 7. METHODS

a. Methods of Treating a Disease or Condition in a Subject

Provided herein are methods of treating a disease or condition in a subject. The method may include administering to the subject a miR-711 inhibitor. In some embodiments, the method includes administering to the subject a miR-711 inhibitor and a TRPA1 inhibitor.

b. Methods of Inhibiting TRPA1 in a Subject

Provided herein are methods of inhibiting TRPA1 in a subject. The method may include administering to the subject a miR-711 inhibitor. In some embodiments, the method includes administering to the subject a miR-711 inhibitor and a TRPA1 inhibitor.

c. Methods of Inhibiting Mir-711 in a Subject

Provided herein are methods of inhibiting miR-711 in a subject. The method may include administering to the subject a miR-711 inhibitor. In some embodiments, the method includes administering to the subject a miR-711 inhibitor and a TRPA1 inhibitor.

### 8. EXAMPLES

### Example 1

### Materials and Methods

Animals. We purchased knockout mice including Trpa1/mice (Stock No: 006401), Tlr7/mice (Stock No:008380) and Trpv1/mice (Stock No: 008336) from Jackson Laboratories. All the knockout mice have C5713/6 background and viable, showing no developmental defects. Immune deficient mice (NOD.CB17-Prkdcscid, Stock No: 001303, 129 background) were also obtained from Jackson Laboratories and used for generating the lymphoma model. We also used Pirt-GCaMP3 mice (Anderson et al., Neurosci. Bull. 2018, 34, 194-199) for calcium imaging. These mice were provided by Dr. Xinzhong Dong of Johns Hopkins University and Andrea Nackley of Duke University. Adult male mice (8-12 weeks), including knockout mice and the same background control mice, as well as some CD1 mice, were used for behavioral studies. Mice were group-housed at Duke University animal facilities on a 12 hr light/12 hr dark cycle at 22±1° C. with free access to food and water. No statistical method was used to predetermine sample size. No randomization was applied to the animal experiments. Sample sizes were chosen based on our previous studies on similar tests (Liu et al., Pain 2016, 157, 806-817; Liu et al., Nat. Neurosci. 2010, 13, 1460-1462). All the animal procedures were approved by the Institutional Animal Care & Use Committee of Duke University. Animal experiments were conducted in accordance with the National Institutes of Health Guide for the Care and Use of Laboratory Animals.

Mouse CTCL Xenograft Model of Chronic Itch. We developed a murine xenograft model of cutaneous T cell lymphoma (CTCL) using immune-deficient mice (NOD.CB17-Prkdcscid, 8-10 weeks old, male). CD4⁺ MyLa cell line was purchased from Sigma (Ca #95051032). The cell line was established from a plaque biopsy of an 82-year old male with mycosis fungoides stage II by inclusion of IL-2 and IL-4 in the culture medium. CTCL was generated via intradermal injection of CD4⁺ Myla cells (1×10⁵ cells/μl, 100 μL) on the nape of the neck. Tumor growth was assessed for 40 days by measurements of tumor diameters.

Mouse DRG cultures. DRGs were collected from young mice (4-6 weeks) of both sexes for primary cultures. These cultures were maintained for less than 3 days for electrophysiological studies.

Human DRGs. Non-diseased human DRGs were obtained from donors through National Disease Research Interchange (NDRI) with permission of exemption from the Duke University Institutional Review Board (IRB). Postmortem L3-L5 DRGs were dissected from 4 donors: 18-year-old male, 54-year-old male, 42-year-old female, and 39-year-old female.

Constructs. The cDNAs of mouse pcDNA3.1-Trpa1, pcDNA3.1-Trpv1, pcDNA3.1-Trpv2, pcDNA3.1-Trpv3, and pcDNA3.1-Trpv4 were kindly provided Dr. Sun Work Hwang from Korea University. Mouse Trpa1 cDNA was subcloned into pCGN-HA backbone (Addgene) using In-Fusion HD Cloning Kit (Clontech Laboratories, CA). Q5 Site-Directed Mutagenesis Kit was used to generate mTrpa1 mutant (M1-M13) based on pCGN-mTrpa1. All primers are listed in TABLE 1.

TABLE 1
List of reagents or resources.

| | | |
|---|---|---|
| REAGENT or RESOURCE | SOURCE | IDENTIFIER |

Antibodies

| | | |
|---|---|---|
| Rabbit polyclonal Anti-TRPA1 (extracellular) | Alomone Labs | Cat# ACC-037, |
| antibody | | RRID:AB_2040232 |
| Sheep polyclonal anti-Digoxigenin Fab fragments | Roche | Roche Cat# |
| antibody, AP Conjugated | | 11093274910, |
| | | RRID:AB_514497 |

Bacterial and Virus Strains

| | | |
|---|---|---|
| NEB ® 5-alpha Competent E. coli | New England | Cat# C2987H |
| | Biolabs | |
| MISSION ® Lenti microRNA Inhibitor (hsa-miR- | Sigma | Cat# HLTUD0996 |
| 711) lentivirus | | |
| Control GFP Adenovirus | Vigene | Cat# CV10001 |
| | Biosciences | |
| Premade Adenovirus for Human miR-711 | Vigene | Cat# VR233338 |
| | Biosciences | |

Chemicals, Peptides, and Recombinant Proteins

| | | |
|---|---|---|
| HC030031 | Sigma | Cat# H4415 |
| A967079 | Sigma | Cat# 5ML0085 |
| AITC | Sigma | Cat# W203408 |
| Capsaicin | Sigma | Cat# M2028 |
| Cannabidiol | Sigma | Cat# C6395 |
| Carvacrol | Sigma | Cat# W224502 |
| GSK1016790A | Sigma | Cat# G0798 |
| Chloroquine | Sigma | Cat# C6628 |
| Histamine | Sigma | Cat# H7250 |
| Hematoxylin Solution, Mayer's | Sigma | MHS16 |
| Eosin Y solution, alcoholic | Sigma | HT110116 |
| Blocking peptide: | Sigma | N/A |
| NH2-FRNELAYPVLTFGQL-COOH | | |
| (SEQ ID NO: 4) | | |
| Mutated peptide: | Sigma | N/A |
| NH2-FRNELAAAVATFGQL-COOH | | |
| (SEQ ID NO: 3) | | |

Critical Commercial Assays

| | | |
|---|---|---|
| In-Fusion ® HD Cloning Kit | Takara Bio | Cat# 121416 |
| Q5 ® Site-Directed Mutagenesis Kit | New England | Cat# E05545 |
| | Biolabs | |
| Streptadvilin agrose beads | ThermoFisher | Cat# 20347 |
| | Scientific | |
| miRNeasy Serum/Plasma Kit | Qiagen | Cat No./ID: 217184 |
| miScript II RT Kit | Qiagen | Cat No./ID: 218160 |
| miScript SYBR Green Kit | Qiagen | Cat No./ID: 218076 |
| miScript miRNA assay for hsa-miR-711 | Qiagen | Cat No./ID: M500017325 |
| miScript miRNA assay for hsa-miR-21 | Qiagen | Cat No./ID: MS00009079 |
| miScript miRNA assay hsa-miR-155 | Qiagen | Cat No./ID: MS00031486 |
| miScript miRNA assay hsa-miR-326 | Qiagen | Cat No./ID: MS00003948 |
| miScript miRNA assay mmu-miR-711 | Qiagen | Cat No./ID: MS00002975 |

Experimental Models: Cell Lines

| | | |
|---|---|---|
| HEK293-hTRPA1 stable cell line | SB Drug | SB-HEK-TRPA1 |
| | Discovery | |
| CD4⁺ Myla cell | Sigma | Cat# 95051032 |
| HuT 102 | ATCC | TIB-162 |
| B16 | ATCC | CRL-6322 |
| CHO cells | ATCC | CRL-9096 |

Experimental Models: Organisms/Strains

| | | |
|---|---|---|
| Mouse:NOD.CB17-Prkdc^{scid} | The Jackson | JAX: 001303 |
| | Laboratory | |
| Mouse:B6;129P- Trpa1^{tm1Kykw} | The Jackson | JAX: 006401 |
| | Laboratory | |
| Mouse:B6.129S1-Tlr7^{tm1Flv} | The Jackson | JAX: 008380 |
| | Laboratory | |
| Mouse:B6.129X1-Trpv1^{tm1Jul} | The Jackson | JAX: 003770 |
| | Laboratory | |

Oligonucleotides

| | | |
|---|---|---|
| mmu-miR-711: gggacccggggagagauguaag | Sigma | N/A |
| (SEQ ID NO: 5) | | |
| mmu-miR-21: uagcuuaucagacugauguuga | Sigma | N/A |
| (SEQ ID NO: 6) | | |
| mmu-miR-155: uuaaugcuaauugugauaggggu | Sigma | N/A |
| (SEQ ID NO: 7) | | |
| mmu-miR-326: gggggcagggccuuugugaaggcg | Sigma | N/A |
| (SEQ ID NO: 8) | | |
| hsa-miR-711: gggacccagggagagagacguaag | Sigma | N/A |
| (SEQ ID NO: 9) | | |
| hsa-miR-642b-3p: agacacauuuggagagggaccc | Sigma | N/A |
| (SEQ ID NO: 10) | | |
| mmu-miR-711(m1): aaaacccggggagagauguaag | Sigma | N/A |
| (SEQ ID NO: 11) | | |
| mmu-miR-711(m2): gggaaaaggggagagauguaag | Sigma | N/A |
| (SEQ ID NO: 12) | | |
| mmu-miR-711(m3): gggacccgaaaagagauguaag | Sigma | N/A |
| (SEQ ID NO: 13) | | |
| mmu-miR-711(m4): gggacccggggaaaaauguaag | Sigma | N/A |
| (SEQ ID NO: 14) | | |
| mmu-miR-711(m5): gggacccggggagagauaaaag | Sigma | N/A |
| (SEQ ID NO: 15) | | |
| mmu-miR-711(m6): aaaaaaaggggagagauguaag | Sigma | N/A |
| (SEQ ID NO: 16) | | |
| mmu-miR-711 core: gggaccc | Sigma | N/A |
| (SEQ ID NO: 1) | | |
| Mmu-miR-711 mutant: aaaaaaa | | |
| (SEQ ID NO: 17) | | |
| mmu-miR-711-bio: | Sigma | N/A |
| gggacccggggagagauguaag-bio | | |
| (SEQ ID NO: 18) | | |
| mmu-miR-711(m6)-bio: | Sigma | N/A |
| aaaaaaaggggagagauguaag-bio | | |
| (SEQ ID NO: 19) | | |
| mmu-miR-711-cy3: | Sigma | N/A |
| gggacccggggagagauguaag-cy3 | | |
| (SEQ ID NO: 20) | | |
| mmu-miR-711(m6)-cy3: | Sigma | N/A |
| aaaaaaaggggagagauguaag-cy3 | | |
| (SEQ ID NO: 21) | | |
| hsa-miR-711 inhibitor: | Shanghai | N/A |
| cuuacgucucucccuggguccc | GenePharm | |
| (SEQ ID NO: 22) | | |
| (DIG)-labeled miRCURY LNA ™ Detection probe | Exiqon | Cat# 612180-330 |
| against hsa-miR-711: cttacgtctctccctgggtc | | |
| (SEQ ID NO: 23) | | |
| (DIG)-labeled miRCURY LNA ™ Detection | Exiqon | Cat# 699004-360 |
| negative control probe: gtgtaacacgtctatacgccca | | |
| (SEQ ID NO: 24) | | |
| Forward primer for subcloning mTrpa1 into pcgn: | Eton | N/A |
| 5′-agcctgggaggaccttctagaatgaagcgcggcttgagg-3 | Bioscience | |
| (SEQ ID NO: 25) | | |
| Reverse primer for subcloning mTrpa1 into pcgn: | Eton | N/A |
| 5′-ctcaccctgaagttctcaggatccctaaaagtccgggtggc-3′ | Bioscience | |
| (SEQ ID NO: 26) | | |
| Reverse primer for mTrpa1 N-terminal deletion: | Eton | N/A |
| 5′-ctcaccctgaagttctcaggatccctaaaagtccgggtggc-3′ | Bioscience | |
| (SEQ ID NO: 27) | | |
| Forward primer for PCGN- mTrpa1 (M1): | Eton | N/A |
| 5′-gctgcagcagccgctggaactagtagtac-3′ | Bioscience | |
| (SEQ ID NO: 28) | | |
| Reverse primer for PCGN- mTrpa1 (M1): | Eton | N/A |
| 5′-agaattgaaggccattccag-3′ | Bioscience | |
| (SEQ ID NO: 29) | | |
| Forward primer for PCGN- mTrpa1 (M2): | Eton | N/A |
| 5′-aattctgctggaataatcgctggaactag-3′ | Bioscience | |
| (SEQ ID NO: 30) | | |
| Reverse primer for PCGN- mTrpa1 (M2): | Eton | N/A |
| 5′-attattccagtagaattgaaggcc-3′ | Bioscience | |
| (SEQ ID NO: 31) | | |
| Forward primer for PCGN- mTrpa1 (M3): | Eton | N/A |
| 5′-atgaggca gcaatagacgctctgaattcatttcca-3′ | Bioscience | |
| (SEQ ID NO: 32) | | |
| Reverse primer for PCGN- mTrpa1 (M3): | Eton | N/A |
| 5′-gagtactactagttccattgattattc-3′ | Bioscience | |
| (SEQ ID NO: 33) | | |
| Forward primer for PCGN- mTrpa1 (M4): | Eton | N/A |
| 5′-tatatggcgtggcaatgtggag-3′ | Bioscience | |
| (SEQ ID NO: 34) | | |
| Reverse primer for PCGN- mTrpa1 (M4): | Eton | N/A |
| 5′-cgctgggatgttgaggaacaag-3′ | Bioscience | |
| (SEQ ID NO: 35) | | |
| Forward primer for PCGN- mTrpa1 (M5): | Eton | N/A |
| 5′-gccccattgctttccttaatcc-3′ | Bioscience | |
| (SEQ ID NO: 36) | | |
| Forward primer for PCGN- mTrpa1 (M5): | Eton | N/A |
| 5′-gctgaaggcatcttggaaattc-3′ | Bioscience | |
| (SEQ ID NO: 37) | | |
| Forward primer for PCGN- mTrpa1 (M6): | Eton | N/A |
| 5′-agcaccgcattgctttccttaatc-3′ | Bioscience | |
| (SEQ ID NO: 38) | | |
| Reverse primer for PCGN- mTrpa1 (M6): | Eton | N/A |
| 5′-gaaggcatcttggaaattc-3′ | Bioscience | |
| (SEQ ID NO: 39) | | |
| Forward primer for PCGN- mTrpa1 (M7): | Eton | N/A |
| 5′-gctgaggcggaatacgcagccctgacctttg-3′ | Bioscience | |
| (SEQ ID NO: 40) | | |
| Forward primer for PCGN- mTrpa1 (M8): | Eton | N/A |
| 5′-gtttagagctgagttggcatac-3′ | Bioscience | |
| (SEQ ID NO: 41) | | |
| Forward primer for PCGN- mTrpa1 (M9): | Eton | N/A |
| 5′-gtttagaaatgaggcggcatac-3′ | Bioscience | |
| (SEQ ID NO: 42) | | |
| Reverse primer for PCGN- mTrpa1 (M8), PCGN- | Eton | N/A |
| Trpa/(M9): 5′-aatggttctaggaaggcatctc-3′ | Bioscience | |
| (SEQ ID NO: 43) | | |
| Forward primer for PCGN- mTrpa1 (M10): | Eton | N/A |
| 5′-aatgagttggaatacccagtcctg-3 | Bioscience | |
| (SEQ ID NO: 44) | | |
| Forward primer for PCGN- mTrpa1 (M11): | Eton | N/A |
| 5′-aatgagttggcatacgcagtcctg-3′ | Bioscience | |
| (SEQ ID NO: 45) | | |
| Forward primer for PCGN- mTrpa1 (M12): | Eton | N/A |
| 5′-aatgagttggcatacccagccctg-3′ | Bioscience | |
| (SEQ ID NO: 46) | | |
| Reverse primer for PCGN- mTrpa1 (M7), PCGN- | Eton | N/A |
| Trpa1(M10), PCGN-Trpa1(M11), PCGN- | Bioscience | |
| Trpa1(M12): 5′-tctaaacaatggttctaggaag-3′ | | |
| (SEQ ID NO: 47) | | |
| Forward primer for PCGN- mTrpa1 (M13): | Eton | N/A |
| 5′-gttggca gccgcagtcgcgacctttgggcagc-3′ | Bioscience | |
| (SEQ ID NO: 48) | | |
| Reverse primer for PCGN- mTrpa1 (M13): | Eton | N/A |
| 5′-tcatttctaaacaatggttctag-3′ | Bioscience | |
| (SEQ ID NO: 49) | | |

Recombinant DNA

| | | |
|---|---|---|
| Plasmid: pCGN-HA | (Tanaka and | Addgene plasmid |
| | Herr, 1990) | #53395 |
| Plasmid: pcDNA3.1-mTrpv1 | Dr. Sun Work | N/A |
| | Hwang from | |
| | Korea | |
| | University | |
| Plasmid: pcDNA3.1-mTrpv2 | Dr. Sun Work | N/A |
| | Hwang from | |
| | Korea | |
| | University | |
| Plasmid: pcDNA3.1-mTrpv3 | Dr. Sun Work | N/A |
| | Hwang from | |
| | Korea | |
| | University | |
| Plasmid: pcDNA3.1-mTrpv4 | Dr. Sun Work | N/A |
| | Hwang from | |
| | Korea | |
| | University | |
| Plasmid: pCGN-mTrpa1 | This paper | N/A |
| Plasmid: pCGN-mTrpa1 (M1) | This paper | N/A |
| Plasmid: pCGN-mTrpa1 (M2) | This paper | N/A |
| Plasmid: pCGN-mTrpa1 (M3) | This paper | N/A |
| Plasmid: pCGN-mTrpa1 (M4) | This paper | N/A |
| Plasmid: pCGN-mTrpa1 (M5) | This paper | N/A |
| Plasmid: pCGN-mTrpa1 (M6) | This paper | N/A |
| Plasmid: pCGN-mTrpa1 (M7) | This paper | N/A |
| Plasmid: pCGN-mTrpa1 (M8) | This paper | N/A |
| Plasmid: pCGN-mTrpa1 (M9) | This paper | N/A |
| Plasmid: pCGN-mTrpa1 (M10) | This paper | N/A |
| Plasmid: pCGN-mTrpa1 (M11) | This paper | N/A |
| Plasmid: pCGN-mTrpa1 (M12) | This paper | N/A |
| Plasmid: pCGN-mTrpa1 (M13) | This paper | N/A |

Behavioral Assessment for Scratching (Itch) and Wiping (Pain). Mice were shaved on the cheek or nape after a brief anesthesia with isoflurane. Before experiments, mice were habituated in small plastic chambers (14×18×12 cm) daily for two days. The room temperature and humidity remained stable for all the experiments. Mice were then briefly removed from the chamber and given an intradermal injection of miRNAs, AITC, histamine, chloroquine (CQ), compound 48/80 (48/80), or peptide with the concentration and volume indicated in the figure legends. After the injection, the number of scratches in 60 min was counted. A scratch was counted when a mouse lifted its hind paw to scratch the shaved region and returned the paw to the floor or to the mouth. A bout of wiping was defined as a continuous wiping movement with a forepaw directing at the area of the injection area (Shimada and LaMotte, Pain 2008, 139, 681-687). Scratching and wiping behavior was videoed for 30 min or 60 min using Sony HDR-CX610 camera. The video was subsequently played back offline and the numbers of scratches and wipes were quantified in a blinded manner.

Pain Tests for Mechanical and Thermal Sensitivity. Mice were habituated to the environment for at least 2 days before the testing. All the behaviors were tested blindly. Inflammatory pain after intraplantar AITC (10 mM, 10 μL) or miR-711 (1 mM, 10 μL) was measured on hind paws. For testing mechanical sensitivity, we confined mice in boxes (14×18×12 cm) placed on an elevated metal mesh floor and stimulated their hind paws with a series of von Frey hairs with logarithmically increasing stiffness (0.16-2.00 g, Stoelting), presented perpendicularly to the central plantar surface. We determined the 50% paw withdrawal threshold by Dixon's up-down method (Dixon, Annu. Rev. Pharmacol. Toxicol. 1980, 20, 441-462). Thermal sensitivity was tested using Hargreaves radiant heat apparatus (Hargreaves et al., Pain 1988, 32, 77-88) (IITC Life Science). For the radiant heat test, the basal paw withdrawal latency was adjusted to 10-15 s, with a cutoff of 25 s to prevent tissue damage.

Evans Blue Extravasation. To examine neurogenic inflammation in a hind paw (Han et al., Neuron 2016, 92, 1279-1293), mice were anesthetized with 5% isoflurane. Evans blue (50 mg/kg body weight) was given intravenously 10 min before neurogenic irritant application. Capsaicin (1 mM, 10 μL), AITC (5 mM, 10 μL), or miR-711 (1 mM, 10 μL) were given by intraplantar injection, and 30 min later mice were sacrificed and plantar tissues were collected and weighted. Evans blue was extracted from the tissues by incubation in 400 μL formamide at 37° C. for 48 hr. Evans blue was quantified by measuring the optical density of the formamide extract at 620 nm. Absorbance was normalized to per gram of tissue weight.

Cell Culture and Transfection. CD4⁺ Myla cell line was cultured in RPMI 1640 media (GIBCO), supplemented with 2 mM Glutamine (GIBCO), 100 U/mL IL-2 (Sigma), 100 U/mL IL-4 (Sigma), and 10% human AB serum (Sigma). HuT 102 cell line was cultured in RPMI 1640 supplemented with pyruvate, HEPES, and 10% (v/v) fetal bovine serum (HyClone). HEK293-hTRPA1 stable cell line was cultured in MEM (GIBCO) containing 2 mM Glutamine, 4 μg/mL Blasticidin, 10% FBS (v/v). B16 and CHO cells were cultured in high glucose (4.5 g/L) Dulbecco's Modified Eagle's Medium (GIBCO) containing 10% (v/v) fetal bovine serum (GIBCO). Culture media were supplemented with 50 units/mL of penicillin and 50 μg/mL streptomycin, and cultures were maintained with 5% CO2 in 37° C. incubator. Transfection (2 μg cDNA) was performed with Lipofectamine™ 2000 Reagent (Invitrogen) at 80% confluency and the transfected cells were cultured in the same media for 48 hr before electrophysiological and biochemical studies.

Generation of Myla Stable Cell Line Expressing miR-711 Inhibitor. CD4⁺ Myla cells were plated at a density of 2×10⁵ cells/mL in 2 mL culture media in 6-well plates. For each well, 20 μL of the MISSION Lenti microRNA Inhibitor (hsa-miR-711) lentivirus (Sigma) with a titer of 2.6×10⁷ Tu/mL were added. Cell mixtures were incubated at 37° C. for 48 hr, washed with PBS three times, and re-suspended in fresh culture media, and 24 hr later, 1 μg/mL puromycin was added to the cells to select stably transduced cell populations. We tried to grow and passage the cells as much as necessary (usually 3 days) and maintained selection pressure by keeping 1 μg/mL puromycin in the medium. After 4 weeks, a large number of the cells were killed; the remaining cells retained the expression of the plasmid, which stably integrates into the genome of the targeted cells. These cells were used for inoculation to generate the CTCL model for testing the effects of miR-711 inhibitor on chronic itch and tumor growth.

Primary Cultures of Mouse and Human Sensory Neurons. DRGs or TGs were removed aseptically from mice (4-6 weeks) and incubated with collagenase (1.25 mg/mL, Roche)/dispase-II (2.4 units/mL, Roche) at 37° C. for 90 min, then digested with 0.25% trypsin for 8 min at 37° C., followed by 0.25% trypsin inhibitor. Cells were mechanically dissociated with a flame polished Pasteur pipette in the presence of 0.05% DNase I (Sigma). DRG cells were plated on glass coverslips and grown in a neurobasal defined medium (with 2% B27 supplement, Invitrogen) with 5 μM AraC and 5% CO2 at 36.5° C. DRG neurons were grown for 24 hr before use.

Non-diseased human DRGs were obtained from donors through National Disease Research Interchange (NDRI) with permission of exemption from the Duke University Institutional Review Board (IRB). Postmortem L3-L5 DRGs were dissected from 4 donors and delivered in ice cold culture medium to the laboratory at Duke University within 24-72 hr of the donor's death. Upon the delivery, DRGs were rapidly dissected from nerve roots and minced in a calcium-free HBSS (GIBCO). Human DRG cultures were prepared as previously reported (Chang et al., Neurosci. Bull. 2018, 34, 4-12; Han et al., Neuron 2016, 92, 1279-1293). DRGs were digested at 37° C. in a humidified CO2 incubator for 120 min with collagenase Type II (Worthington, 290 units/mg, 12 mg/mL final concentration) and dispase II (Roche, 1 unit/mg, 20 mg/mL) in PBS with 10 mM HEPES, pH adjusted to 7.4 with NaOH. DRGs were mechanically dissociated using fire-polished pipettes, filtered through a 100 μm nylon mesh and centrifuged (500 g for 5 min). The pellet was resuspended, plated on 0.5 mg/mL poly-D-lysine-coated glass coverslips, and grown in Neurobasal medium supplemented with 10% FBS, 2% B-27 supplement, and 1% penicillin/streptomycin. Whole-cell patch-clamp recordings in small (<50 mm) human DRG neurons were conducted at room temperature using patch pipettes with resistances of 3-4 MΩ.

Whole-Cell Patch Clamp Recordings in HEK293 Cells, CHO Cells, and DRG Neurons. Whole-cell patch clamp recordings were performed at room temperature using an Axopatch-200B amplifier (Axon Instruments) with a Digidata 1440B (Axon Instruments). In this study, we only examined small-diameter mouse DRG neurons (<25 μM) and small-diameter human DRG neurons (<55 μM). The patch pipettes were pulled from borosilicate capillaries (World Precision Instruments) using a P-97 Flaming/Brown micropipette puller (Sutter Instrument). Pipette resistance was 4-6 MΩ for whole-cell and outside-out recording. For inward current recordings in mouse and human DRG neurons, HEK293 cells, and CHO cells, the internal solution contains (in mM): 140 CsCl, 10 EGTA, 10 HEPES, and 2 Mg-ATP, adjusted to pH 7.3 with CsOH. Whole cell recordings were performed in an extracellular solution that contains (in mM): 140 NaCl, 5 KCl, 2 MgCl₂, 10 HEPES, and 10 glucose, adjusted to pH 7.4 with NaOH and osmolarity to 300-310 mOsm. To record miR-711 and AITC evoked action potentials, the amplifier was switched to the current-clamp mode. Action potential recordings were performed in small-diameter DRG neurons, with the following solutions: i) internal pipette solution contains: 126 mM K-gluconate, 10 mM NaCl, 1 mM MgCl₂, 10 mM EGTA, 10 mM HEPES and 2 mM Na-ATP, adjusted to pH 7.4 with KOH and osmolarity to 295-300 mOsm), and ii) extracellular solution contains: 140 mM NaCl, 5 mM KCl, 2 mM MgCl₂, 2 mM CaCl₂, 10 mM HEPES, 10 mM glucose, adjusted to pH 7.4 with KOH.

Calcium and sodium ion permeability experiments were conducted to compare the effects of miR-711 and AITC (Wang et al., J. Biol. Chem. 2008, 283, 32691-32703). Inward current recordings were made in TRPA1-expressing HEK293 cells with an Axopatch 200B amplifier and digitized with a Digidata 1440A digitizer, acquired with Clampex 10.6, and analyzed with Clampfit 10.6 (Axon Instruments, Union City, Calif.). Data were sampled at 10 kHz, and filtered at 2 kHz. The resistance of the pipettes was 4-5MΩ. For measurement of ion permeability, the membrane potential was ramped from +80 mV to −80 mV (1 V/s). The recording solutions for ion permeability experiments are, i) bath solution: 145 mM NaCl, 10 mM HEPES, and 2 mM EDTA, or 128 mM CaCl₂ with 10 mM HEPES (pH 7.4 with NaOH), and ii) pipette solution: 145 mM CsCl, 2 mM MgATP, 10 mM HEPES (pH 7.4 with CsOH). The I/V curve and reversal potential were analyzed as previously demonstrated (Wang et al., J. Biol. Chem. 2008, 283, 32691-32703).

To test the effects of miR-711 on calcium channels, calcium currents were recorded in mouse DRG neurons (Andrade et al., Nat. Neurosci. 2010, 13, 1249-1256). Calcium current was evoked by a 40-ms step depolarization to −10 mV from the holding potential of −80 mV, using i) external solution (mM): 135 mM TEA-C1, 1 mM CaCl₂, 10 mM HEPES, 4 mM MgCl₂ and 0.1 μM TTX, adjusted to a pH of 7.4 with TEA-OH, and ii) the pipette solution: 126 mM CsCl, 5 mM Mg-ATP, 10 mM EGTA and 10 mM HEPES, adjusted to a pH of 7.3 with CsOH.

Inside-Out and Outside-Out Patch Clamp Recordings on HEK293 Cells. For inside-out recordings in TRPA1-expressing HEK293 cells, pipette resistance was 8-10 MΩ and, the internal solution contains: 126 mM K-gluconate, 10 mM NaCl, 1 mM MgCl₂, 10 mM EGTA, 10 mM HEPES, and 2 Na-ATP (adjusted to pH 7.3 with KOH, osmolarity to 295-300 mOsm). The recordings were performed at room temperature in a bath solution (intracellular side) of 140 mM NaCl, 10 mM EGTA, 5 mM KCl, 2 mM CaCl₂, 1 mM MgCl₂, 10 mM HEPES, 10 mM glucose, adjusted to pH 7.4 with NaOH and osmolarity to 300-310 mOsm (Park et al., Neuron 2014, 82, 47-54). For comparison, outside-out recordings were also performed in TRPA1-expressing HEK293 cells in an extracellular solution of 140 mM NaCl, 5 mM KCl, 2 mM MgCl₂, 10 mM HEPES, and 10 mM glucose (adjusted to pH 7.4 with NaOH and osmolarity to 300-310 mOsm). The internal solution contains 140 mM CsCl, 10 mM EGTA, 10 mM HEPES, and mM 2 Mg-ATP, adjusted to pH 7.3 with CsOH. Currents were low-pass filtered at 2 kHz and digitized at a sampling rate of 10 kHz with Digidata-1440A (Axon Instruments). The pClamp10 (Axon Instruments) software was used during experiments and data analysis. Opening and closing transitions of single channels were detected by using 50% of the threshold criterion. All events were carefully checked before the analysis. When superimposed openings were observed, the number of channels in the patch was estimated from the maximal number of superimposed openings. The single-channel open probability (Po) was determined using the following equation: Po=T′/T, where T′ is the total open time for a patch over time T.

Ca²⁺ Imaging. Ca ²⁺ imaging was conducted in mouse DRG and TG neurons from Pirt-GCaMP3 mice (Anderson et al., Neurosci. Bull. 2018, 34, 194-199) at room temperature. The imaging buffer includes 140 mM NaCl, 10 mM D-(+)-Glucose, 1 mM MgCl2, 2 mM CaCl₂, 5 mM KCl, 10 mM HEPES, pH=7.4, osmolarity=320 mOsm/L. Calcium signals were measured using green emitted light in a 3 s interval. Ca ²⁺ signal amplitudes were presented as ΔF/F₀=(Fₜ−F₀)/F₀ as ratio of fluorescence difference (Fₜ−F₀) to basal value (F₀). The average fluorescence intensity in the baseline period was taken as F₀. Ca ²⁺ imaging was also analyzed in HEK293-TRPA1 cell line after loading cells with 2 mM fura2-AM (Invitrogen) for 40 min in the Ca²⁺ imaging buffer. Ca²⁺ imaging protocol was a ratio metric method with 340/380-nm wavelength light for dual excitation. Data were presented as ΔR/R₀, determined as the fraction ΔR (Rₜ−R₀) of the increase of a given ratio over baseline ratio (R₀).

Live Cell Labeling and Immunocytochemistry in Mouse DRG Neurons. Disassociated DRG neurons were plated on Poly-D-Lysine coated coverslips and cultured in Neurobasal medium supplemented with B27 for 24 hr. Cells were incubated with 10 μM Cy3-labeled miR-711 or miR-711 (m6) in extracellular cell solution for 15 min at 37° C. with 5% CO2. Then the coverslips were washed and incubated with TRPA1 primary antibody (Alomone lab, rabbit, 1:100) at 4° C. for 1 hr. The cells on coverslips were incubated with secondary antibody conjugated to FITC (1:100; Jackson ImmunoResearch, West Grove, Pa.) and examined under a Leica SP5 inverted confocal microscope.

RNA Pull-Down Assay and Immunoblotting. hTRPA1-expressing HEK293 cells of equal amount (5×10⁶) were plated onto 60 mm dishes, and 24 hr later, these cells were incubated with biotin-conjugated miR-711 or miR-711 (m6) in extracellular solution containing 140 mM NaCl, 5 mM KCl, 2 mM CaCl₂, 1 mM MgCl₂, 10 mM HEPES, and 10 mM glucose, adjusted to pH 7.4 for 15 min at 37° C. with 5% CO₂. For crosslinking, 1% formaldehyde (vol/vol) was added at 37° C. for 10 min, then 12.5% glycine was applied to stop crosslinking by 5 min incubation at room temperature. Then the cells were collected and sonicated with ultrosonic probe sonicator (80% output, 5 s on and 10 s off) in 1 mL lysis buffer (1% Triton X-100, 50 mM Tris-HCl, 150 mM NaCl, 5 mM EDTA, pH7.4) on ice for 2 min. We then centrifuged the lysate at 13,000 rpm for 10 min at 4° C. and took 50 μL supernatant as lysate control. The remaining supernatant was added 20 μL streptadvilin agrose beads and incubated overnight. The pellets were collected after centrifuge at 6000 rmp for 30 s. For competing assay, miR-711 (10-50 μM) or mutant miR-711 (m6, 10-50 μM) were added 15 min before the incubation with biotin-conjugated miR-711 (10 μM). For immunoblotting, the lysates or beads were incubated in SDS-PAGE loading buffer for 30 min at 50° C., and supernatant was collected after centrifugation at 13000 rpm and decrosslink at 99° C. for 20 min. The samples were separated on an SDS-PAGE gel, transferred, and probed with TRPA1 antibody (1:1000; Alomone labs). The immunoreactive bands were detected with horseradish peroxidase-conjugated secondary antibody, visualized with enhanced chemiluminescence (Thermo scientific, Pittsburgh, Pa.), and quantified with Image-Pro Plus software (Media Cybernetics, Bethesda, Md.). Each experiment was repeated at least three times.

Fluorescent In Situ Hybridization (FISH). Digoxigenin (DIG)-labeled miRCURY LNA Detection probe against hsa-miR-711 “CTTACGTCTCTCCCTGGGTC,” (SEQ ID NO: 23) and negative control “GTGTAACACGTCTATACGCCCA” (SEQ ID NO: 24) were used for in situ hybridization. After transcardiac perfusion with 4% paraformaldehyde, mouse tumor and skin tissues were dissected. FISH was carried out according to the manufacturer's guide. Tissue sections were cut in cryostat at 14-μm thickness. The sections were fixed with 4% paraformaldehyde for 10 min at room temperature and acetylated at room temperature for 10 min. Probes were diluted with Hybridization buffer to 50 nM and hybridized at 55° C. overnight. Sections were then incubated with alkaline phosphatase conjugated anti-DIG (1:3500; Roche) overnight at 4° C. After washing, the in situ signals were developed with Fast Red substrate. For quantification, four or five tumor sections from each mouse were selected, and three mice were analyzed in each group. To quantify the percentage of labeled cells, the number of positive cells within one field were divided by the total area of the field to obtain the density of cells. Images were analyzed with Image-Pro Plus5.1 (Media Cybernetics) or Adobe PhotoShop.

miRNA Measurement by Quantitative Real-Time RT-PCR (qPCR). Total RNA was isolated from serum of CTCL mice or adenovirus-treated mice using Qiazol Lysis Reagent (QIAGEN) together with miRNeasy Serum/Plasma Kit (Park et al., Neuron 2014, 82, 47-54). All RNA samples were immediately used or kept at −80° C. until further processing. To convert mature miRNA into cDNA, 6 μl of total RNA solution were reverse transcribed using the miScript II RT Kit (including polyadenylation of miRNAs and reverse transcription using an oligo-dT that binds to a universal RT-sequence). Specific miRNA levels were quantified by qPCR using miScript SYBR Green Kit including miScript miRNA assay for hsa-miR-711, hsa-miR21, hsamiR155, and hsa-miR-326, together with the universal RT primer, according to the manufacturer's protocol (CFX96 Real-Time system, Bio-Rad). Relative quantities of miRNAs were calculated using the Ct value after normalization to control miRNAs. Caenorhabditis elegans miRNA-39 (cel-miRNA-39) was included as spiked-in control for extracellular miRNA.

Computer Simulations. To elucidate the binding modes and interaction energies of specific miRNA sequences to TRPA1, we generated the structural model of the complex between TRPA1 and the miRNA sequence GGGACCC (SEQ ID NO: 1), which appears to be an essential for TRPA1 activation and itch induction. We generated the initial structural model of TRPA1 starting from the coordinates of the human isoform of the protein, which has been recently solved via cryo-electron microscopy at 4.24 Å resolution (pdb-id: 3j9p) (Paulsen et al., Nature 2015, 520, 511-517). We refined the structural features of TRPA1 by introducing the missing extracellular loops using MODELER (Webb and Sali, Methods Mol. Biol. 2014, 1137, 1-15). The ion channel structure was then optimized by means of a short discrete molecular dynamics simulation (DMD) (Dokholyan et al., Fold. Des. 1998, 3, 577-587; Shirvanyants et al., J. Phys. Chem. B 2012, 116, 8375-8382), which consisted of 5×10⁵ time steps at temperature 0.5 kcal/(mol k_{B}) corresponding to ˜25 ns and ˜250 K, respectively. The quality of the DMD-generated lowest energy conformation of TRPA1 was assessed using Gaia (http://redshift.med.unc.edu/chiron/login.php), our in-house developed software, which compares the intrinsic structural properties of our computational model to high-resolution crystal structures (Kota et al., Bioinformatics 2011, 27, 2209-2215). Our modeled TRPA1 structure was well within the bounds of high-resolution crystal structure parameters in Gaia database and, thus, further adopted for computational studies in the presence of miRNA.

In a second stage, using iFoldRNA, an in-house developed methodology for RNA structure prediction with near atomic resolution accuracy (Krokhotin et al., Bioinformatics 2015, 31, 2891-2893; Sharma et al., Bioinformatics 2006, 22, 2693-2694), we generated the structural model of the miRNA711 core sequence GGGACCC (SEQ ID NO: 1), which was randomly positioned at a distance of 25 Å from the extracellular TRPA1 surface. In order to explore the GGGACCC sequence's ability to bind TRPA1, we employed the replica-exchange sampling method (Zhou et al., Proc. Natl. Acad. Sci. 2001, 98, 14931-14936) implemented in DMD (RexDMD) (Dokholyan et al., Fold. Des. 1998, 3, 577-587; Shirvanyants et al., J. Phys. Chem. B 2012, 116, 8375-8382). In RexDMD, multiple simulations (replicas) of the same system are performed in parallel at different temperatures, and are coupled through a Monte Carlo-based algorithm for the exchange of temperatures at recurrent time intervals. This simulation scheme allows to overcome energy barriers and to efficiently explore the binding free energy surface of the GGGACCC/TRPA1 system. We used 18 parallel replicas with temperatures ranging from 0.3 to 0.6 kcal/(mol k_{B}) (corresponding to ˜175 K and 310 K, respectively) with increments of either 0.01 or 0.02 kcal/(mol kB). In each system the position of miRNA711ps' center of mass was constrained within a maximum radius of 15 Å from TRPA1 extracellular surface, and every replica is simulated for 2 million time steps (i.e., ˜100 ns).

With the aim of isolating the most relevant structural model of the GGGACCC/TRPA1 complex, we retrieved all configurations of the system in which the estimated interaction energy between the two species was equal or lower than −75 kcal/mol (i.e., peak of left shoulder in the interaction energy distribution). We then clustered the retrieved high-affinity GGGACCC/TRPA1 conformational ensemble according to the root mean square deviation (RMSD) of the miRNA sequence's phosphorus atoms, using the unweighted pair group method with centroid (UPGMC) as implemented in the Python SciPy library (https://scipy.org). For the clustering analysis we imposed a cutoff of 4.24 Å (i.e., resolution of TRPA1 structural coordinates) to distinguish two distinct GGGACCC/TRPA1 conformations. The entries of the most populated cluster (˜4% of the isolated conformational space) were analyzed to explore the binding mode of GGGACCC to TRPA1, and the lowest estimated binding energy conformation of the ensemble (i.e., −87 kcal/mol) was chosen as the representative structure of the GGGACCC/TRPA1 complex.

In order to assess the stability of the identified GGGACCC (SEQ ID NO: 1) sequence's binding mode we performed five independent, 4.5×10⁵ steplong DMD simulations at temperature 0.3 kcal/(mol k_{B}), corresponding to ˜20 ns and ˜175 K, respectively. We monitored the fluctuation of GGGACCC sequence's bound conformation by measuring the RMSD of its phosphorus atoms, as well as the standard deviation of the interatomic distances between GGGACCC and TRPA1 residues within 5 Å of each nucleobase. The RMSD of GGGACCC phosphorus atoms averaged around 1.75 Å. Similarly, the fluctuations of the inter-atomic distances between GGGACCC and TRPA1 were far below the resolution of the TRPA1 cryo-electron microscopy structure (i.e., the value beyond which two atoms cannot be distinguished as different), indicating that identified binding mode is stable and consistent with the experimental structural data.

Quantification and Statistical Analysis. All data were expressed as mean±SEM, as indicated in the figure legends. Statistical analyses were completed with Prism GraphPad 6.0. Biochemical and behavioral data were analyzed using two-tailed Student's t test (two groups), One-Way or Two-Way ANOVA followed by post hoc Bonferroni test. Electrophysiological data were tested using one-way ANOVA (for multiple comparisons) or two-tailed Student's t test (two groups), as shown in our previous studies (Park et al., Neuron 2014, 82, 47-54; Xu et al., Nat. Med. 2015, 21, 1326-1331). The criterion for statistical significance was p<0.05.

### Example 2

### miR-711 Elicits Pruritus Via Specific Core Sequence and TRPA1

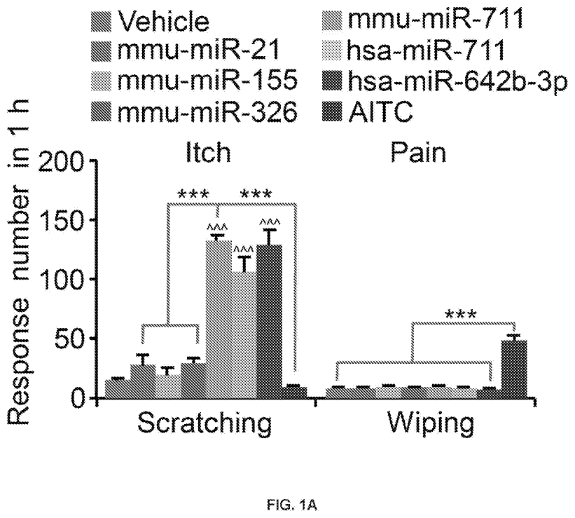

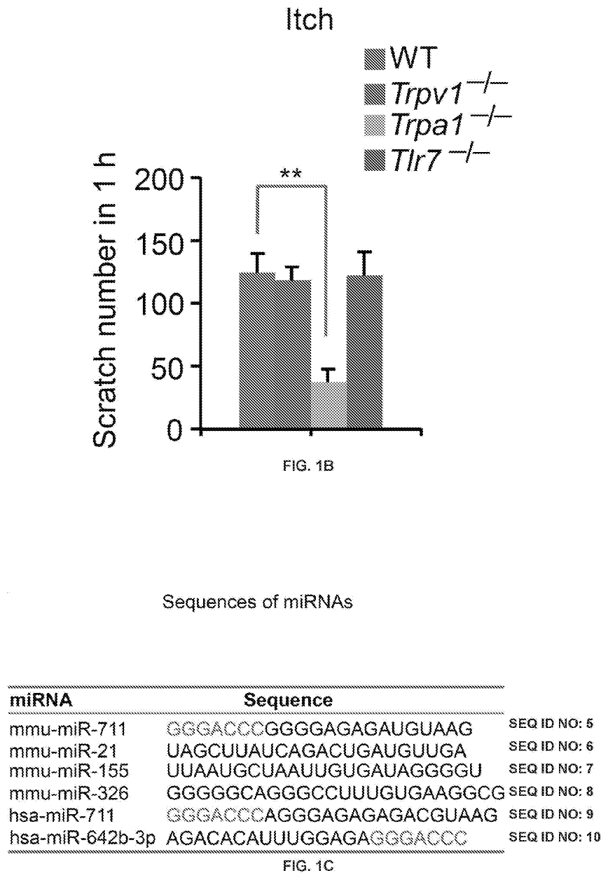

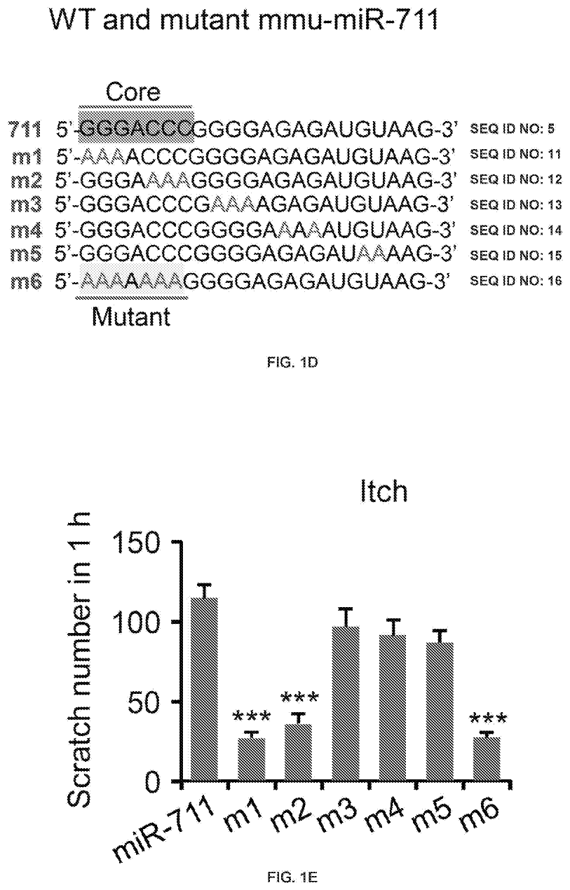

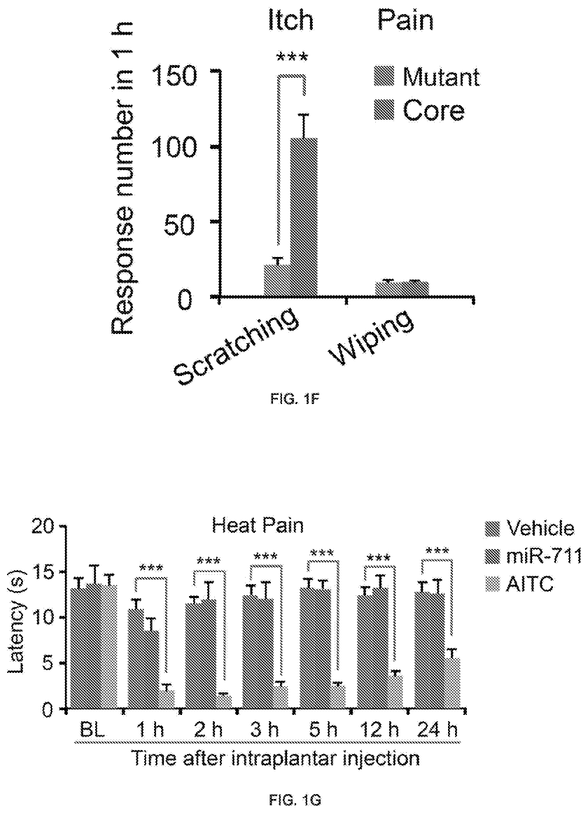

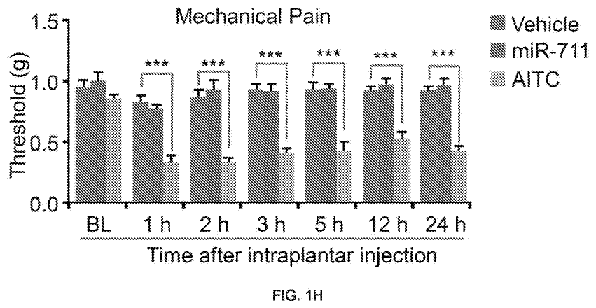

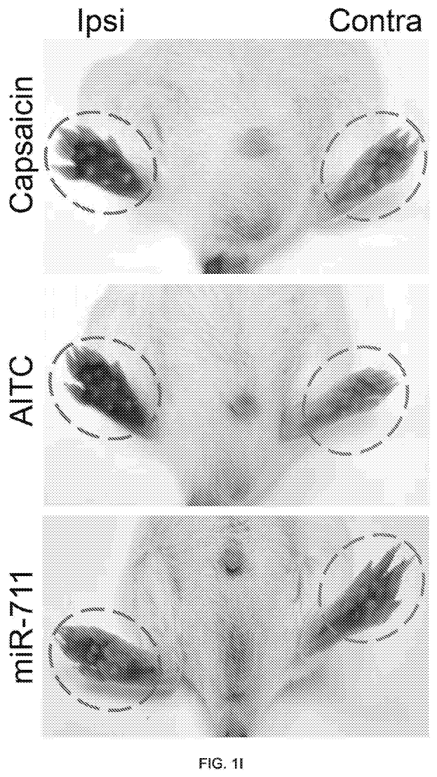

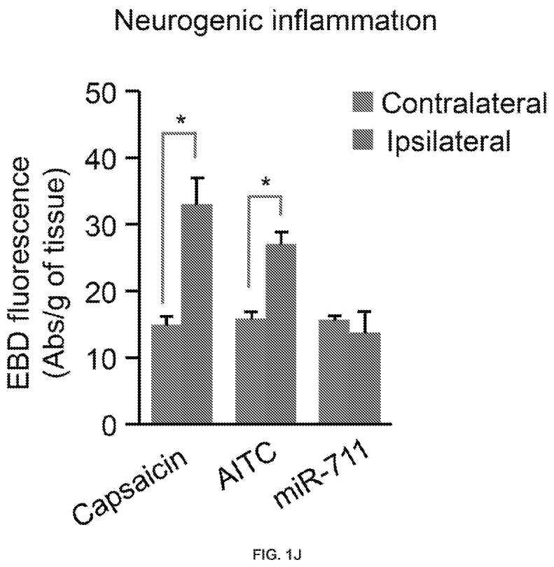

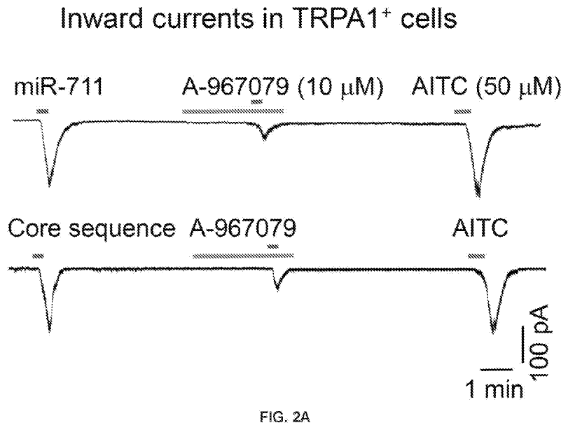

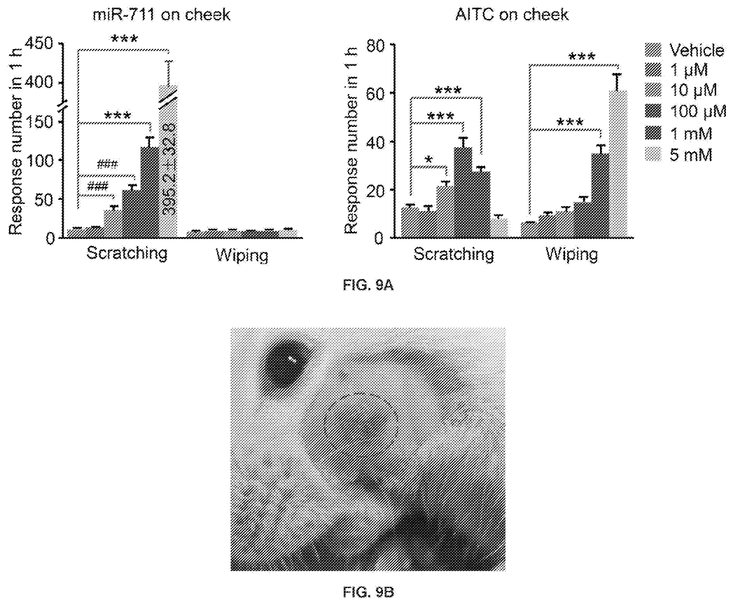

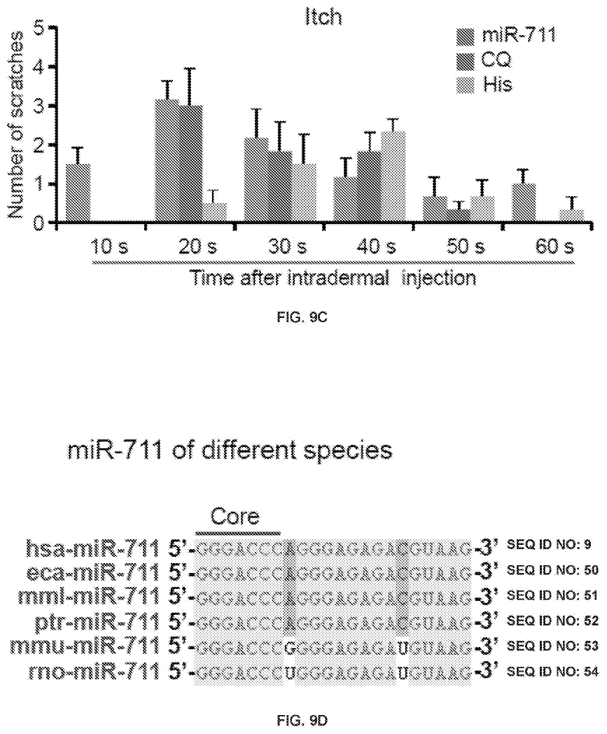

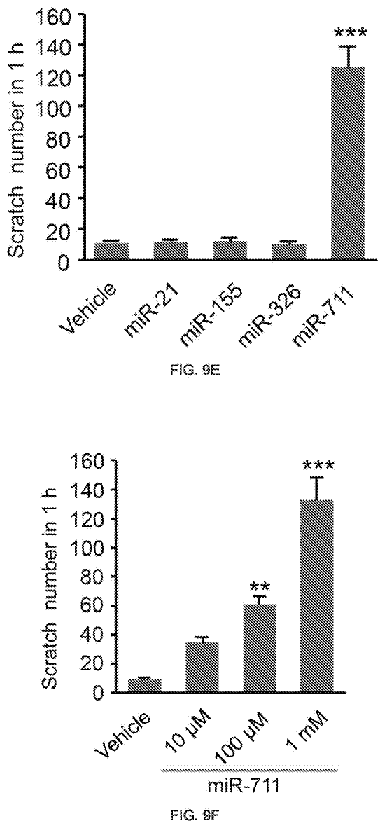

We searched for miRNAs that are dysregulated in patients with lymphoma. Upregulations of miR-21, miR-155, miR-326, and miR-711 were reported in skin biopsies from the lymphoma patients. Next, we tested whether the dysregulated miRNAs are capable of inducing itch or pain in naive animals following intradermal injection (1 mM, 5 μL) using the cheek model that can distinguish pain versus itch (Shimada and LaMotte, Pain 2008, 139, 681-687). Notably, among 4 miRNAs we tested, only miR-711, but not miR-21, miR-155, and miR-326, evoked marked scratching in naive mice (FIG. 1A). Pruritus evoked by intradermal miR-711 is dose dependent: mild but significant scratching was evident at 0.01 and 0.1 mM (p<0.001, FIG. 9A). At the highest dose (5 mM, 5 μL) we tested, intradermal miR-711 resulted in a very strong scratching with 395.2±32.8 bouts per hour. This severe pruritus resulted in skin lesion at the injection site (FIG. 9A-FIG. 9B). Thus, miR-711 is a highly potent pruritogen. The onset of miR-711-induced pruritus was very rapid, with a shorter latency than that induced by the classic pruritogens chloroquine (CQ) and histamine (FIG. 9C), suggesting that miRNA may trigger pruritus through different mechanisms. Despite severe pruritus, intradermal cheek injection of miR-711 failed to evoke pain (wiping) at all the concentrations we tested (FIG. 1A-FIG. 9A). Neither did intradermal miR-21, miR-155, and miR-326 elicit wiping behavior (FIG. 1A). We also examined miRNA-induced itch using the back model, as the cheek skin and back skin are differentially innervated by trigeminal ganglion (TG) and DRG sensory neurons. Intradermal injection of miRNA-711 (0.01-1 mM, 10 μL) on the nape of neck also induced dose-dependent scratching, whereas miR-21, miR-155, and miR-326 had no effects (FIG. 9E-FIG. 9F). TRPA1 and TRPV1 are expressed by pruriceptive and nociceptive DRG neurons and regulate acute and chronic pruritus. We reasoned that miR-711 might trigger itch via direct or indirect activation of TRP channels on sensory neurons. miR-711-induced acute itch was abrogated in Trpa1^{-/-} but not Trpv1^{-/-} mice (FIG. 1B). Pruriceptive neurons also expressed TLR7, which induces pain or itch by coupling to TRPA1. However, miR-711-induced pruritus was unaltered in Tlr7^{-/-} mice (FIG. 1B). Thus, miR-711 evokes itch via TRPA1 but not TRPV1 and TLR7.

Does miRNA induce itch via specific sequence? FIG. 1C shows the sequences of different miRNAs we tested in FIG. 1A. A comparison of mouse and human miR-711 sequence revealed that mmu-miR-711 and hsa-miR-711 contain the same core sequence GGGACCC and both miRNAs from different species were able to evoke pruritus (FIG. 1A and FIG. 9D). A miRNA database (miRBase) search showed that hsa-miR-642b-3p also contains the core sequence GGGACCC, and consistently, intradermal hsa-miR-642b-3p resulted in pruritus too (FIG. 1A). Like mouse miR-711, hsa-miR-711 and hsa-miR-642b-3p failed to elicit wiping at the concentration that can produce scratching (FIG. 1A).

To determine the specific sequence of miR-711 that is critical for pruritus, we generated 6 mutants of miR-711 (m1 to m6) by converting several nucleotides to adenosine (FIG. 1D) and tested their effects on pruritus. Mutations on the first seven nucleotides in m1, m2, and m6 resulted in marked reduction in scratching, suggesting that GGGACCC is the core sequence for eliciting pruritus (FIG. 1E). Importantly, this core sequence was sufficient to elicit scratching but not wiping, whereas the mutant oligonucleotides (AAAAAAA, SEQ ID NO: 17) did not affect pain and itch (FIG. 1F).

Next, we examined whether AITC and miRNA-711 produce distinct pain or itch. Intradermal and cheek administration of AITC at high concentrations (5 and 10 mM, 5 μL) induced wiping but not scratching. Interestingly, low concentrations of AITC (10 and 100 μM) induced mild itch but no pain, whereas a medium concentration (1 mM) induced both scratching and wiping (FIG. 1A and FIG. 9A). This finding suggests that AITC produces both pain and itch in a dose-dependent manner. We also tested mechanical and thermal pain sensitivity in a hind paw. Intraplantar injection of miR-711, at the concentration that can elicit itch (1 mM), failed to induce heat hyperalgesia and mechanical allodynia. In contrast, intraplantar AITC elicited marked hyperalgesia and allodynia (FIG. 1G-FIG. 1H).

Neurogenic inflammation is a unique form of inflammation arising from the release of inflammatory mediators from primary afferent neurons. Intraplantar injection of capsaicin (1 mM) and AITC (5 mM) elicited robust neurogenic inflammation in hind paws, as revealed in the Evans blue test. However, intraplantar administration of miR-711 (1 mM) failed to elicit neurogenic inflammation (FIG. 1I-FIG. 1J).

Collectively, these results indicate that miR-711 and AITC differently regulate pain, itch, and neurogenic inflammation.

### Example 3

### miR-711 Activates TRPA1 in Heterologous Cells to Elicit Inward Currents and Single Channel Activities

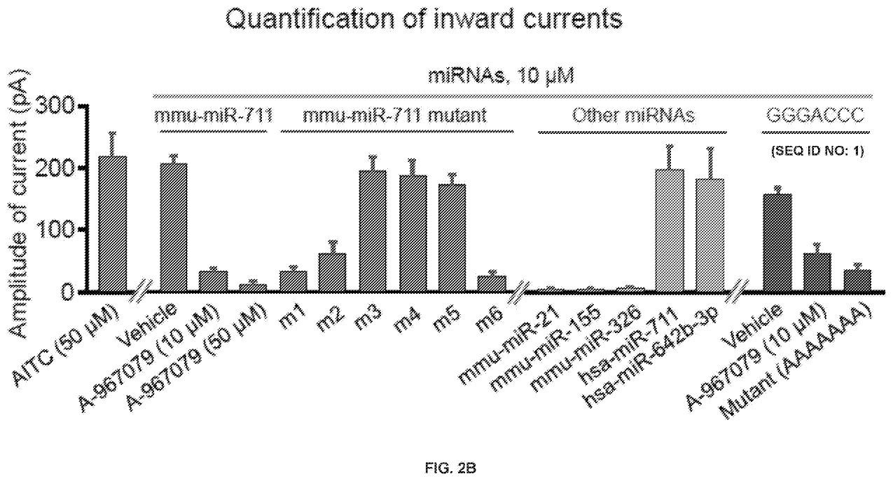

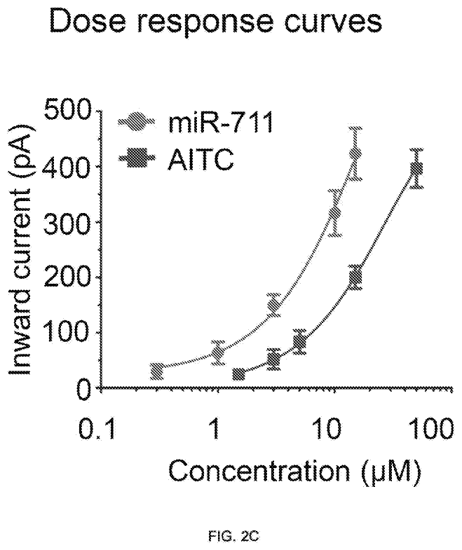

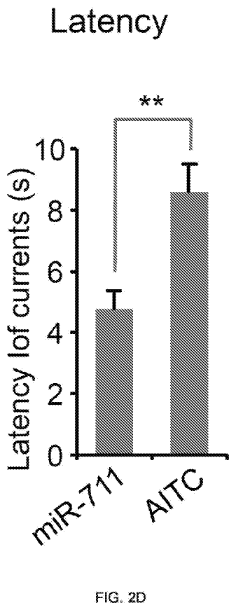

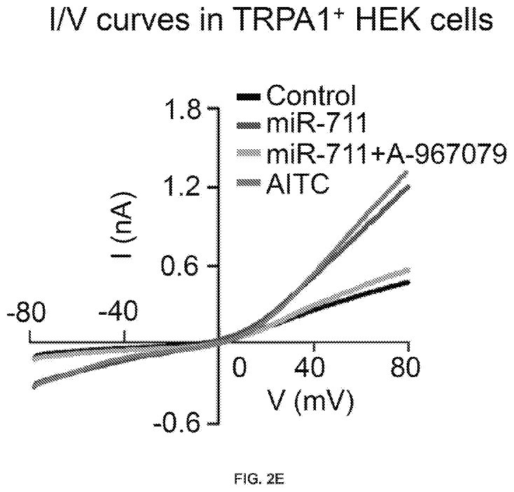

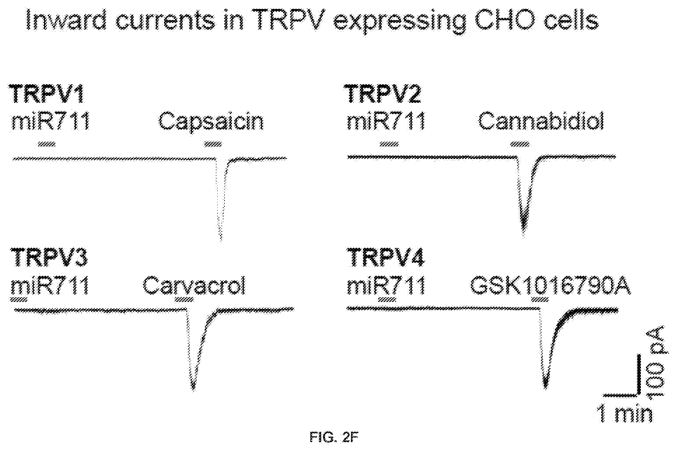

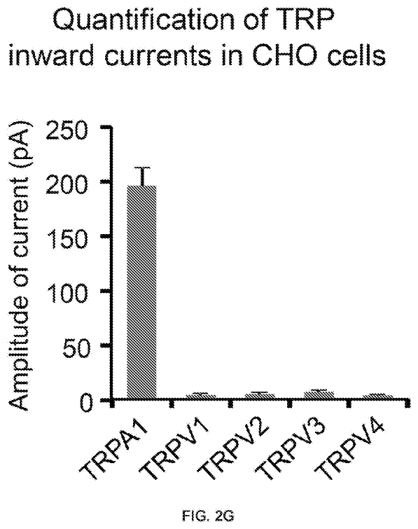

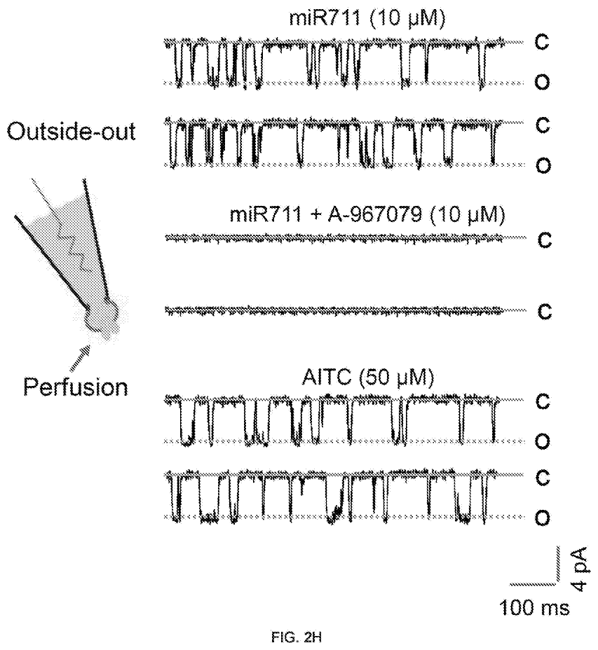

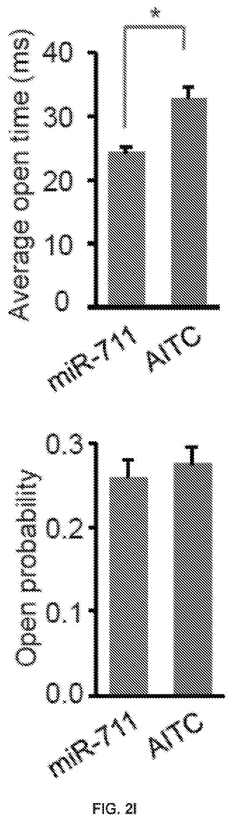

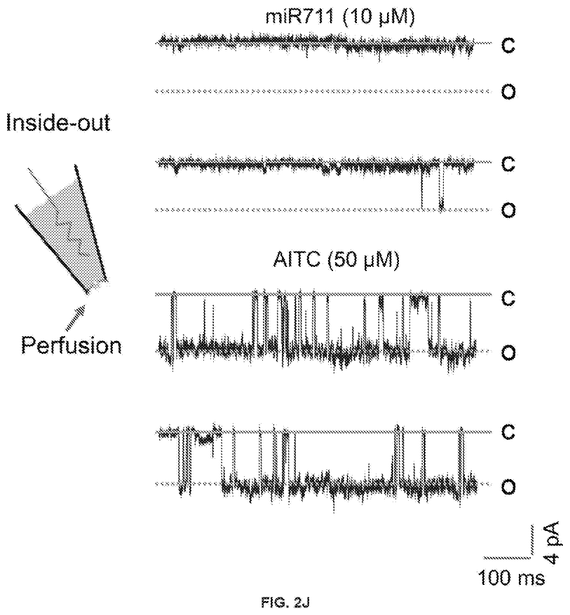

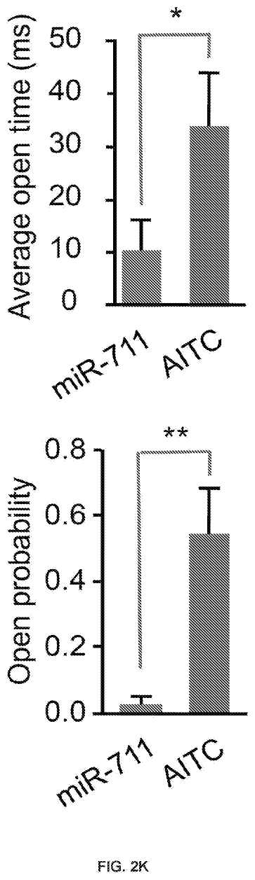

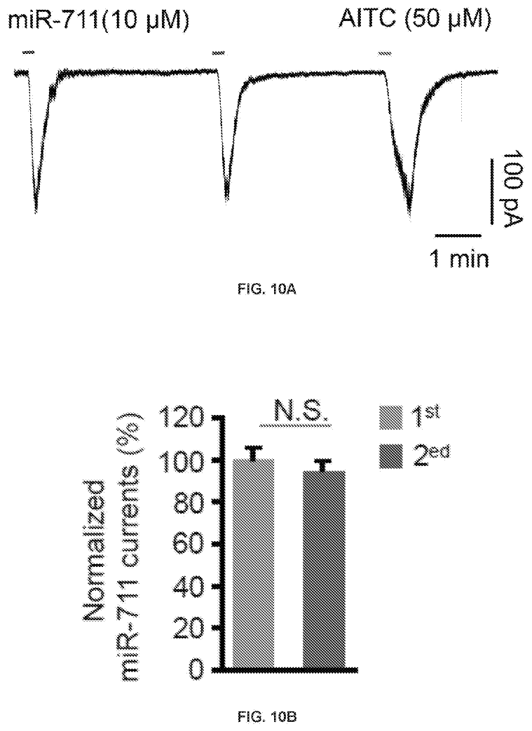

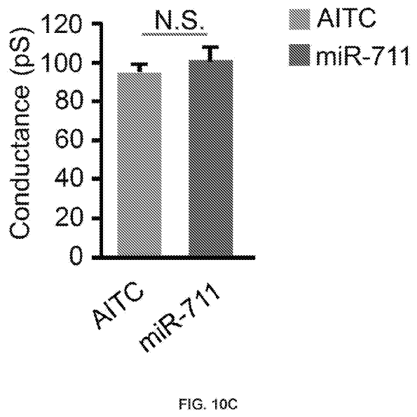

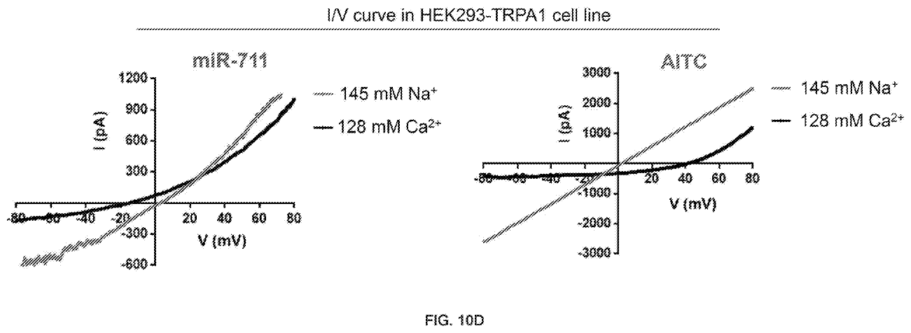

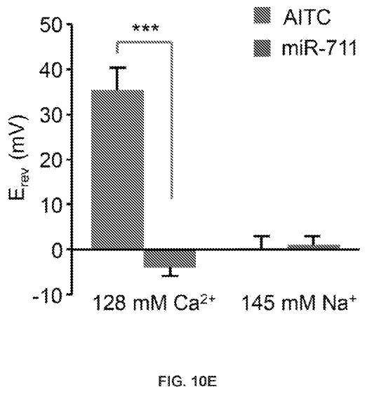

To investigate whether miR-711 directly activates TRPA1, we assessed the function of TRPA1 by conducting patch-clamp recordings in heterologous cells. Bath application of miR-711, but not miR-21, miR-155, and miR-326 (10 μM), produced inward currents in Trpa1-expressing HEK293 cells (FIG. 2A-FIG. 2B). AITC (50 μM) also induced inward currents in these cells (FIG. 2A-FIG. 2B). The second application of miR-711 did not induce obvious desensitization of TRPA1 (FIG. 10A-FIG. 10B). A967079, a selective TRPA1 antagonist (10 and 50 μM) dose-dependently blocked the miR-711-evoked currents (FIG. 2A-FIG. 2B). A dose-response analysis revealed that miR-711 is more potent than AITC for inducing TRPA1 activation, as indicated by a left-shift of the dose-response curve (FIG. 2C). Of interest, the latency of miR-711-induced inward currents is shorter than that of AITC (FIG. 2D), implicating a faster action of miR-711. The miR-711-evoked current had a current-voltage relationship consistent with TRPA1 activation, with a reversal potential of 0 mV and outward rectification (FIG. 2E). By contrast, miR-711 failed to trigger inward currents in CHO cells that express Trpv1, Trpv2, Trpv3, and Trpv4, suggesting that miR-711 specifically acts on TRPA1 (FIG. 2F-FIG. 2G).

To validate that miR-711 directly activates TRPA1 on the cell surface, we carried out single channel recordings in HEK293 cells expressing TRPA1. Outside-out patch recordings showed that bath application of miR-711 and AITC on the extracellular surface each elicited single-channel opening events (FIG. 2H). The miR-711-induced single channel activity was completely blocked by A967079 (FIG. 2H). Interestingly, the duration of the miR-711-evoked single channel opening (average open time) but not the open probability, and conductance was significantly shorter (p<0.05) than that evoked by AITC (FIG. 2I and FIG. 10C). Inside-out patch recordings in Trpa1-expressing HEK293 cells showed that bath application of AITC but not miR-711 on the intracellular surface elicited single-channel opening events (FIG. 2J-FIG. 2K). Thus, unlike AITC, miR-711 activates TRPA1 on the extracellular side.

### Example 4

### miR-711 Activates a Subpopulation of Sensory Neurons Reminiscent of Pruriceptors

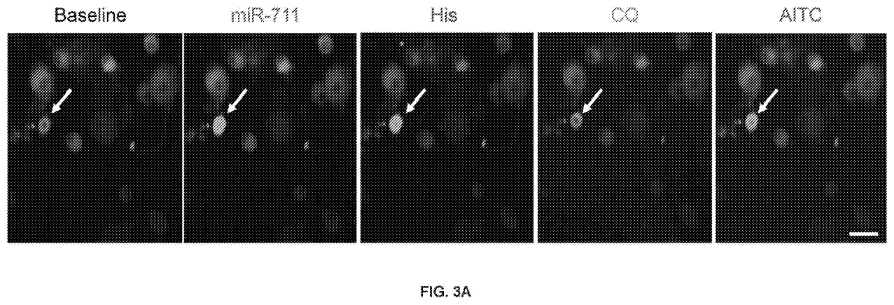

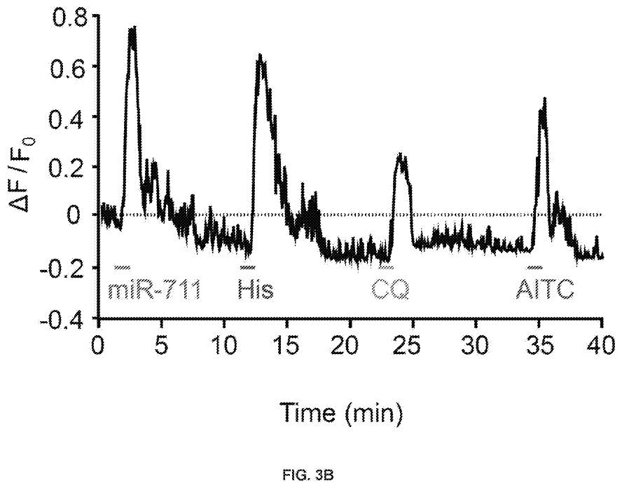

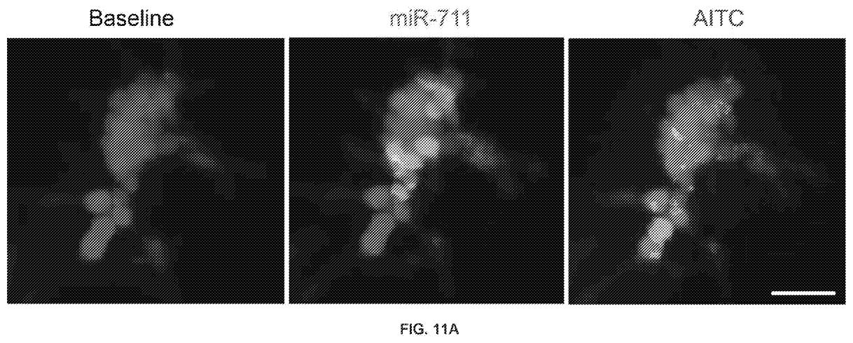

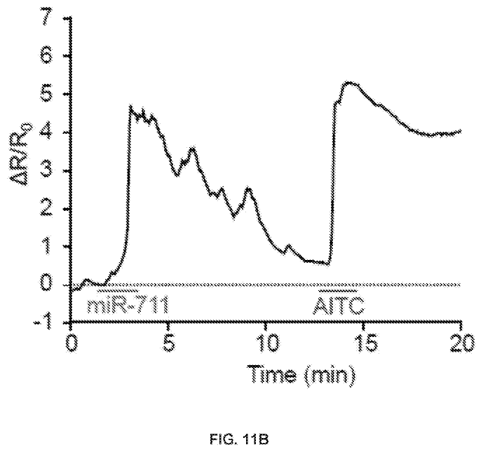

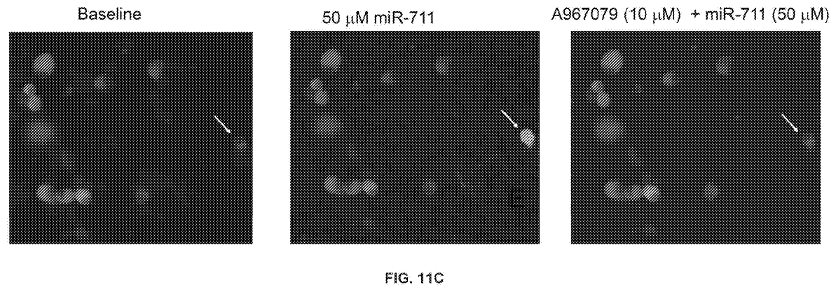

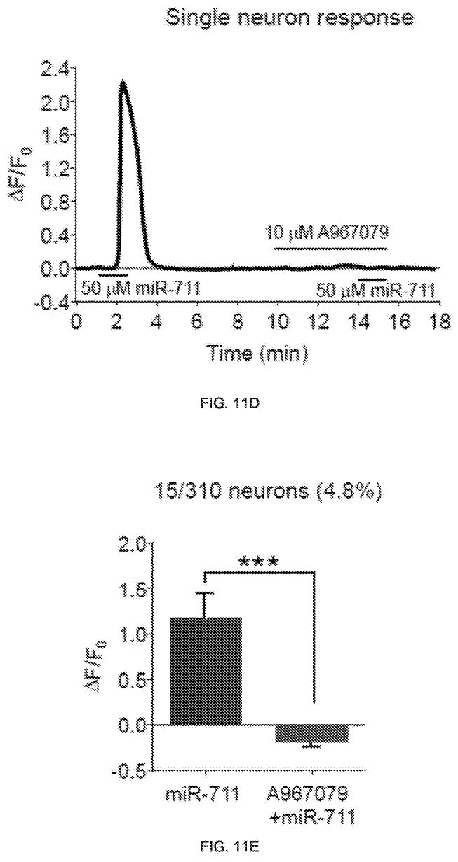

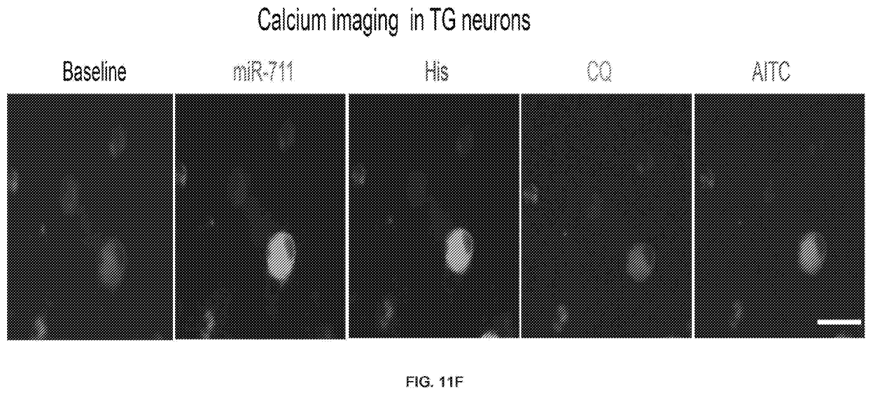

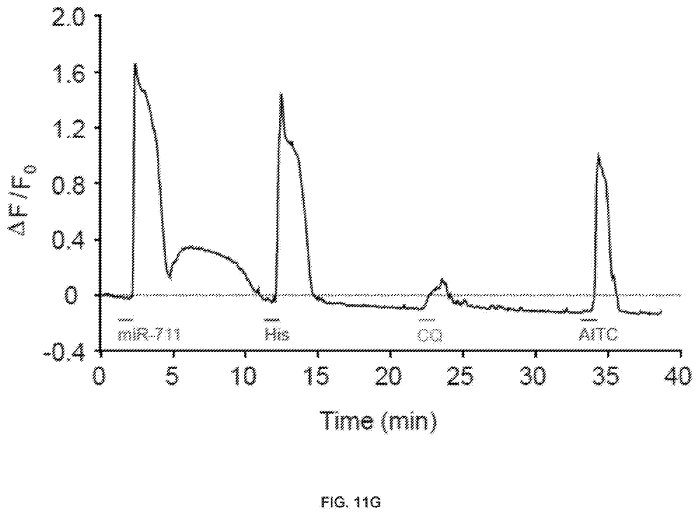

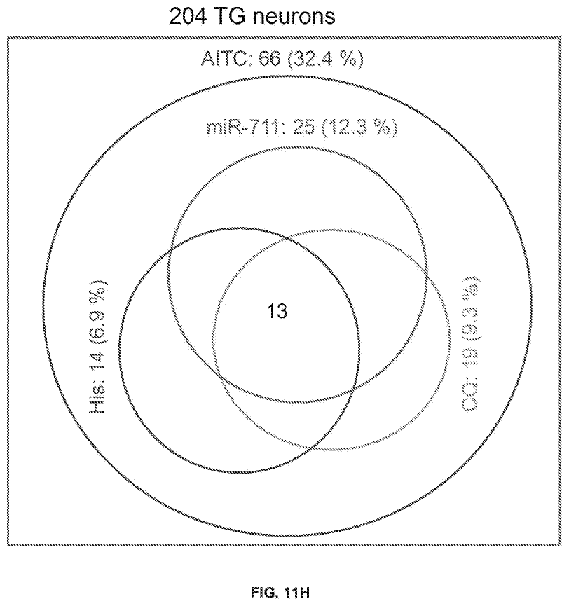

To determine the neuronal population activated by miR-711, we performed calcium imaging on cultured DRG neurons isolated from Pirt-GCaMP3 mice (Anderson et al., Neurosci. Bull. 2018, 34, 194-199). We found that 10 μM miR-711 did not evoke meaningful calcium signaling, although this concentration was sufficient to induce inward currents in TRPA1-expressing HEK293 cells. At 50 μM, miR-711 caused Ca²⁺ increase in 3.9% DRG neurons (n=544 neurons from 3 mice, FIG. 3A-FIG. 3B). After miR-711 stimulation, we sequentially stimulated the same DRG neurons with histamine (500 μM), chloroquine (CQ, 1,000 μM), and AITC (200 μM). In 544 neurons we analyzed, 5.5%, 5.1%, and 22.6% neurons showed responses to histamine, CQ, and AITC, respectively (FIG. 3C). For the miR-711-resposive neurons, majority of them also showed responses to CQ (66.7%) and histamine (61.9%), and all of them responded to AITC (FIG. 3A-FIG. 3C). As expected, miR-711 (50 μM) also evoked calcium responses in TRPA1-expressing HEK293 cells (FIG. 11A-FIG. 11B). Notably, A967079 completely blocked the miR-711-evoked calcium responses in DRG neurons, confirming a specific activation of TRPA1 by miR-711 (FIG. 11C-FIG. 11E).

We also examined calcium responses in TG neurons isolated from Pirt-GCaMP3 mice. Interestingly, compared with DRG neurons, TG neurons showed greater responses to miR-711, pruritogens, and AITC: 12.3% (25/204) of TG neurons responded to miR-711 (50 μM), and 6.9%, 9.3%, and 32.4% neurons exhibited respective responses to histamine, CQ, and AITC. Among the miR-711-responsive neurons, the majority of them also showed responses to CQ (76%) and histamine (52%), and all (100%) to AITC (FIG. 11F-FIG. 11H). Collectively, our calcium imaging data indicate that miR-711 activates a subset of TRPA1⁺ sensory neurons in mice.

### Example 5

### miR-711 and AITC Cause Distinct Activation of TRPA1 in Mouse DRG Neurons

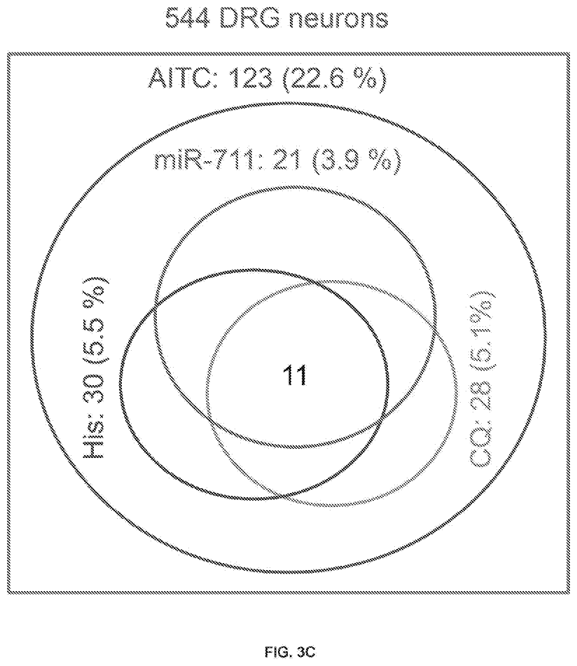

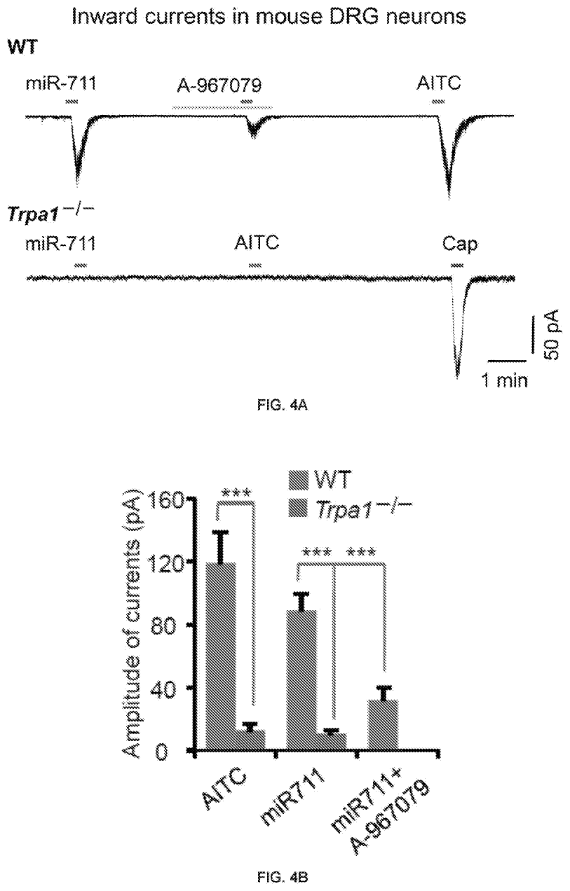

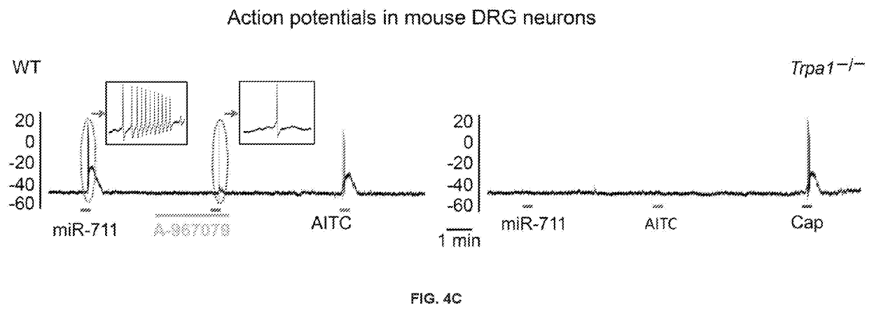

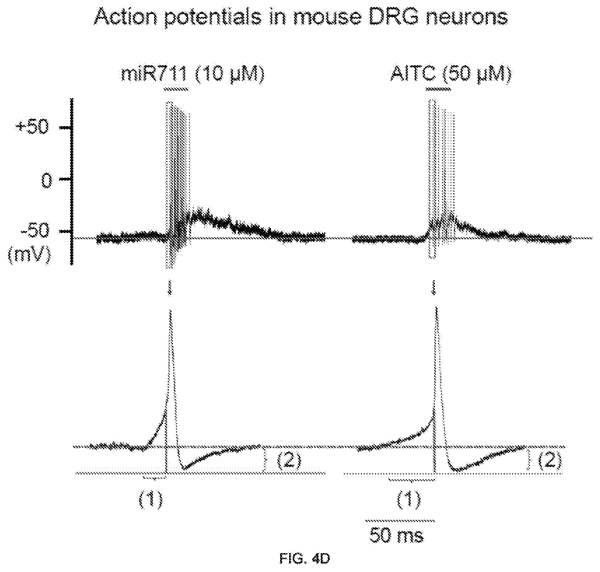

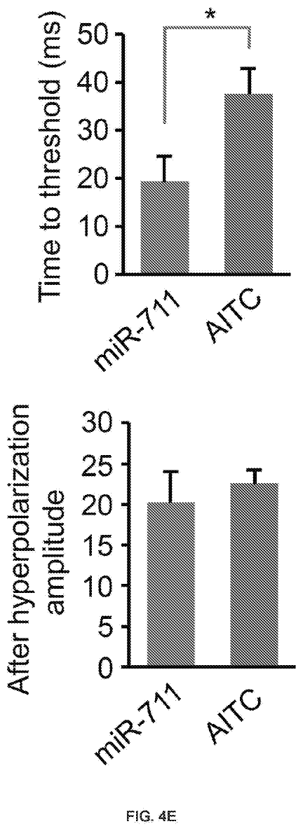

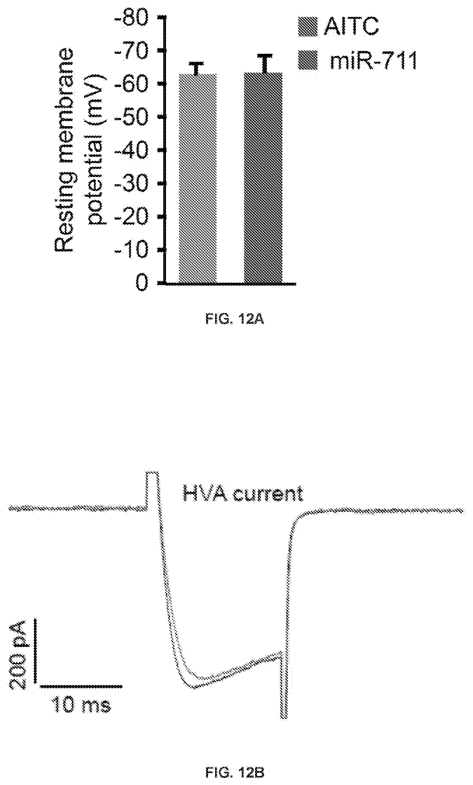

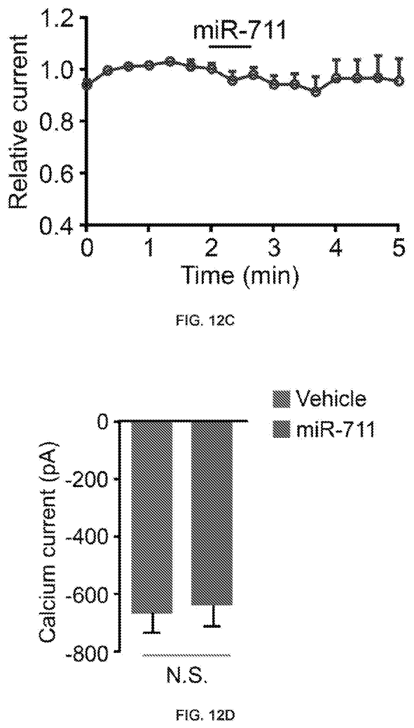

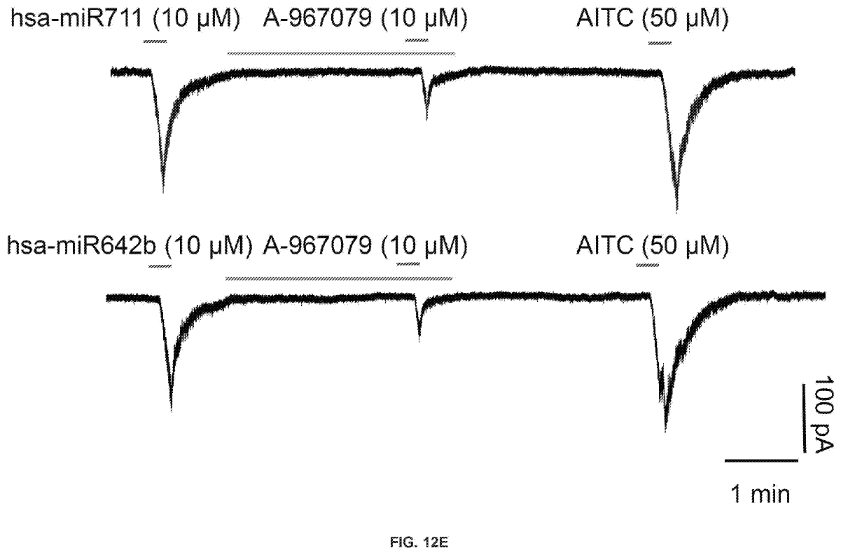

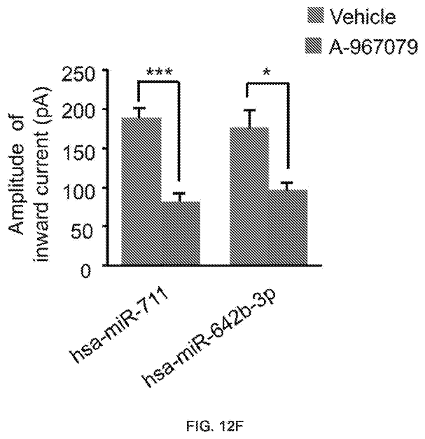

To further assess distinct neuronal activation by miR-711 and AITC, we conducted electrophysiology to record inward currents and action potentials in small-diameter DRG neurons (<25 μm). Exposure of dissociated DRG neurons to exogenous miR-711 (10 μM) induced rapid inward currents, which were blocked by A-967079 and abolished in Trpa1 knockout mice (FIG. 4A-FIG. 4B). miR-711 (10 μM) also induced action potentials in small-diameter DRG neurons, and this excitation was lost in TRPA1-deficient neurons (FIG. 4C-FIG. 4D), suggesting that miR-711 is sufficient to excite sensory neurons via TRPA1. Notably, 16.7% (15/90) and 11.5% (15/130) small-diameter neurons responded to AITC and miR-711, respectively, with inward currents or action potentials. Thus, miR-711-responding neurons could be a subset of TRPA1⁺ neurons. Additional action potential analysis revealed that compared with AITC, miR-711 elicited action potentials with a shorter duration, but the after-hyperpolarization of the action potentials did not differ after these treatments (FIG. 4D-FIG. 4E). It was shown that AITC increased calcium permeability. I/V and reversal potential analysis revealed that compared to AITC, miR-711 had lower permeability to Ca²⁺ but similar permeability to Na⁺ in Trpa1-expressing HEK293 cells (FIG. 10D-FIG. 10E). The resting membrane potentials (RMPs) of the recorded neurons are near −60 mV, indicating their healthy conditions (FIG. 12A). Our data suggest that distinct TRPA1 activation by miR-711 and AITC may underlie their distinct sensory behaviors (itch versus pain).

Since miR-711 at 10 mM induced inward current but no calcium response in DRG neurons, we also assessed whether miR-711 would inhibit calcium channel activities. No evidence was found to support this notion: miR-711 at 10 μM did not alter calcium currents in DRG neurons (FIG. 12B-FIG. 12D).

We also tested the actions of miRNAs in human DRG neurons from donors (Chang et al., Neurosci. Bull. 2018, 34, 4-12). Given the shared core sequence of mouse and human miR-711, we predicted that human DRG neurons should also respond to hsa-miR-711. As shown in FIG. 12E, hsa-miR-711 (10 μM) evoked TRPA1-dependent inward currents in human DRG neurons. Furthermore, hsa-miR-642b-3p, which contains the GGGACCC core sequence (SEQ ID NO: 1) and is capable of inducing scratching in mice (FIG. 1A and FIG. 1C), induced similar inward currents on human DRG neurons as hsa-miR-711 (FIG. 12E-FIG. 12F). The fact that certain miRNAs can activate human sensory neurons highlights a translational potential of this study.

### Example 6

### miR-711 Core Sequence Binds to TRPA1 at Specific Residues

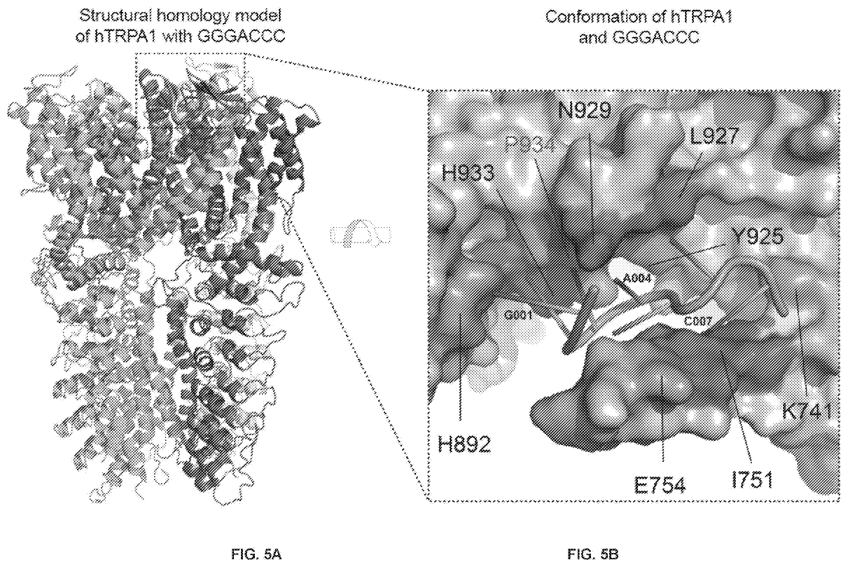

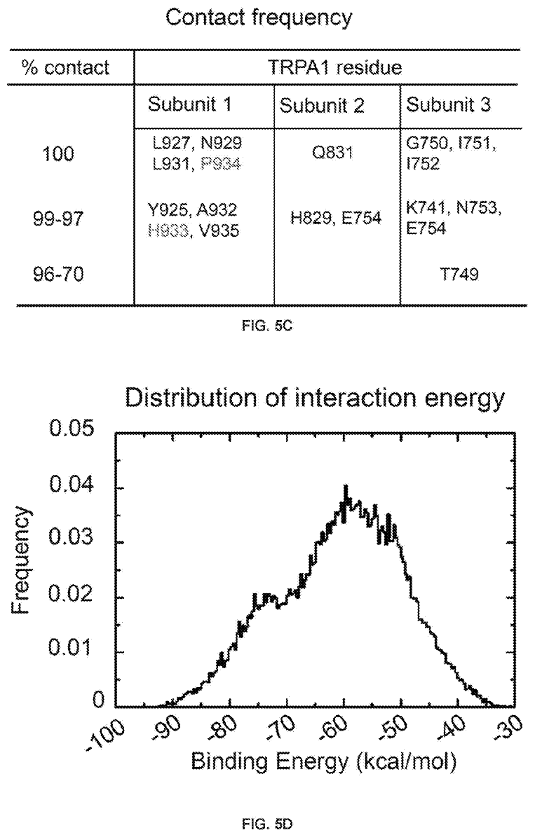

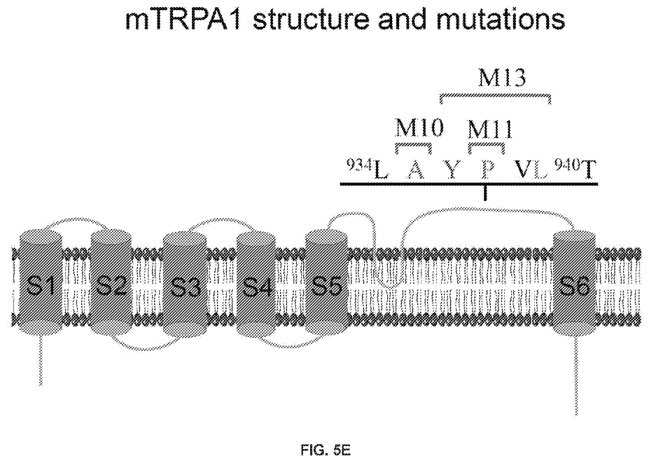

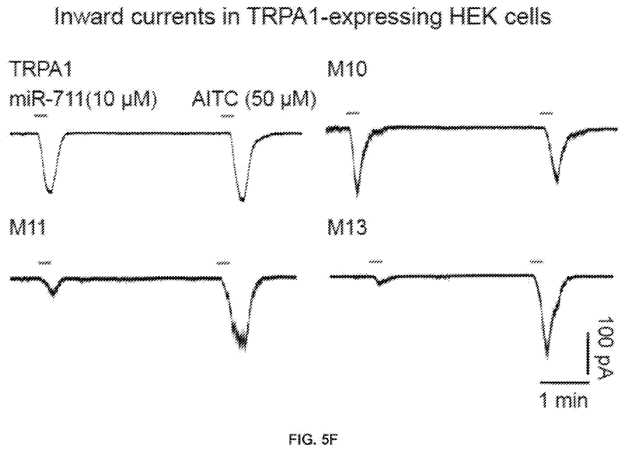

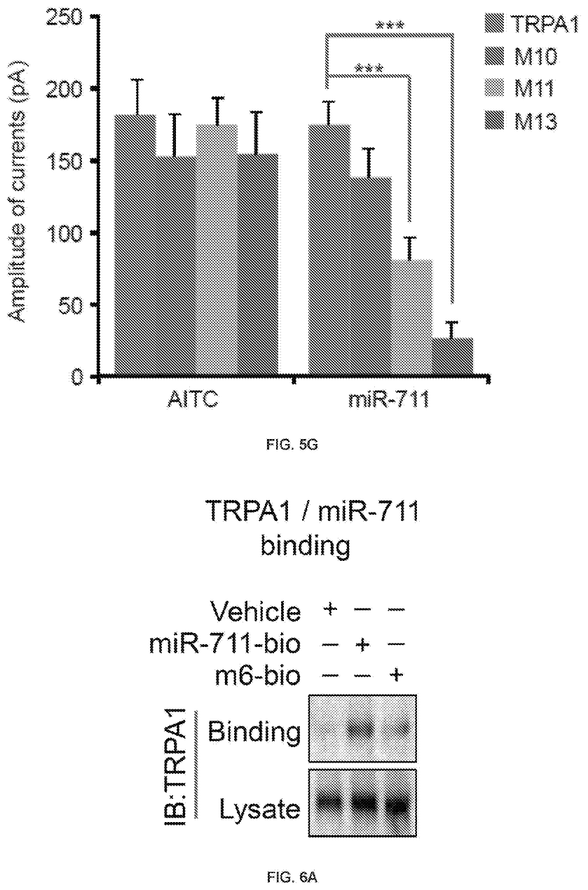

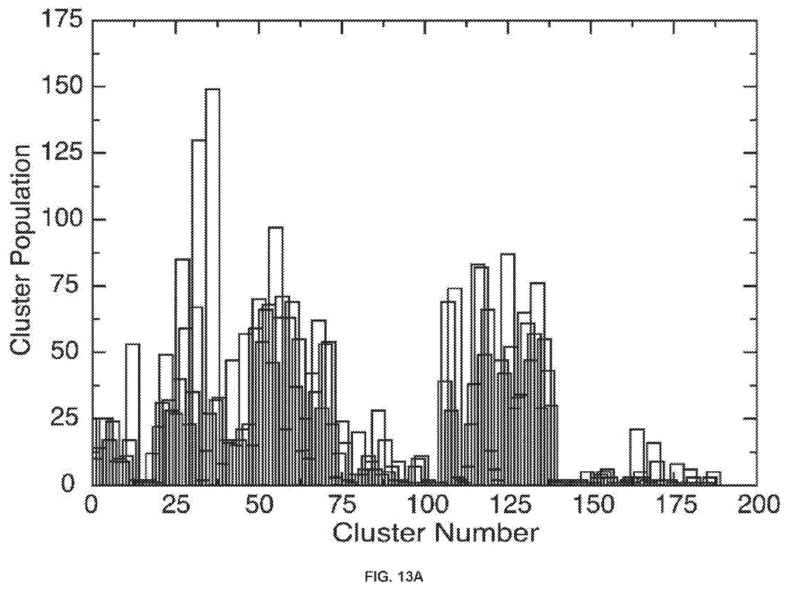

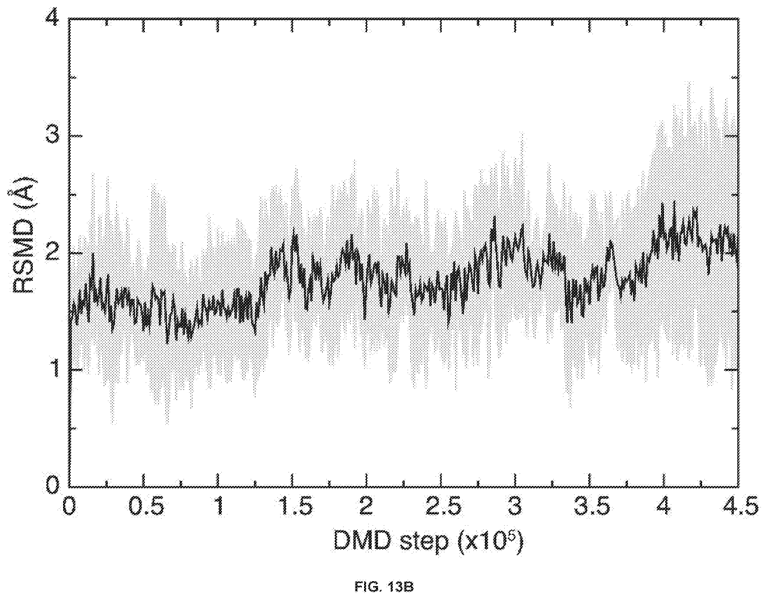

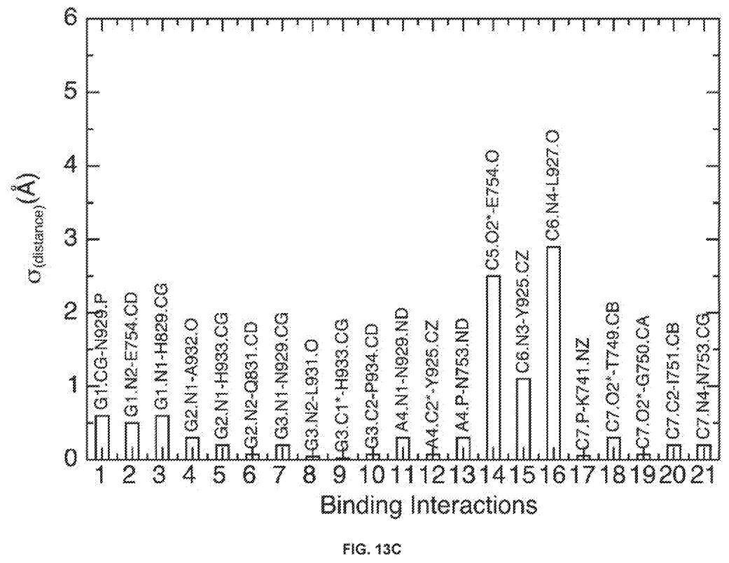

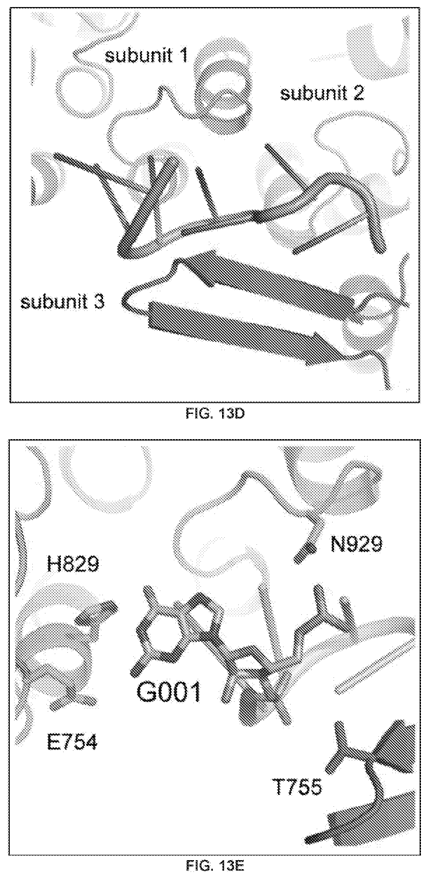

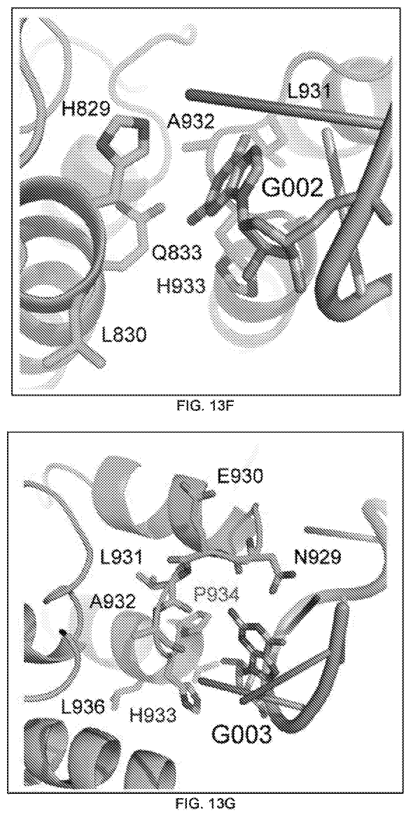

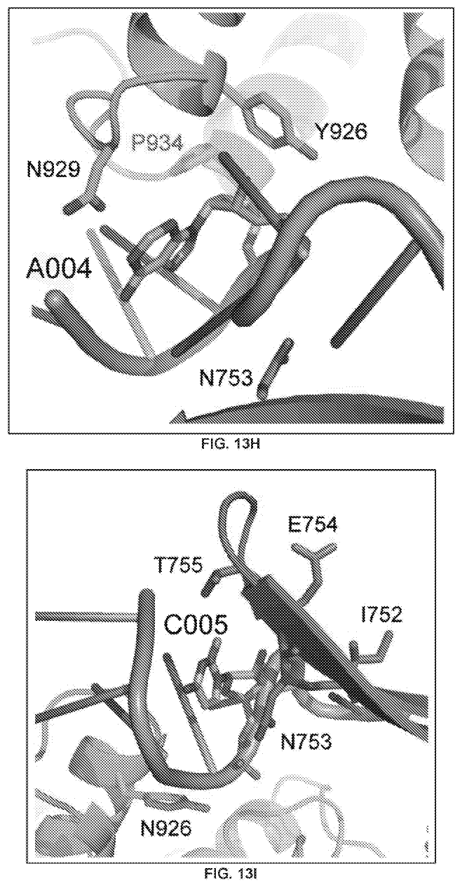

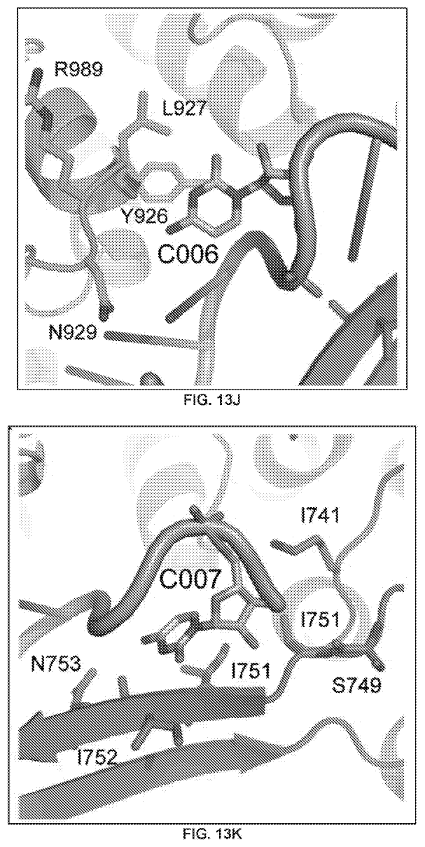

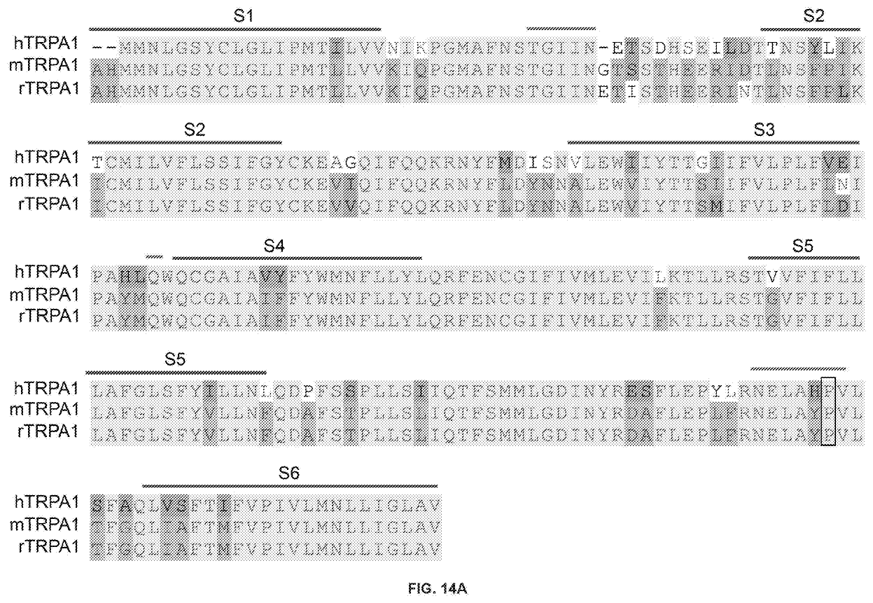

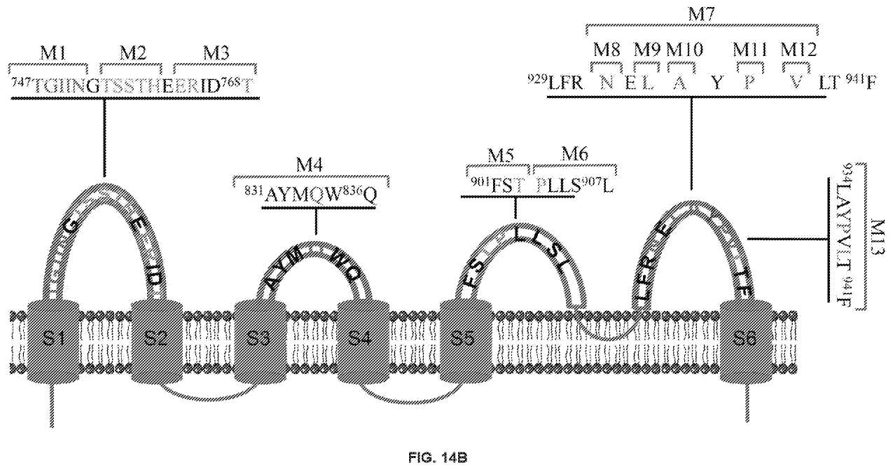

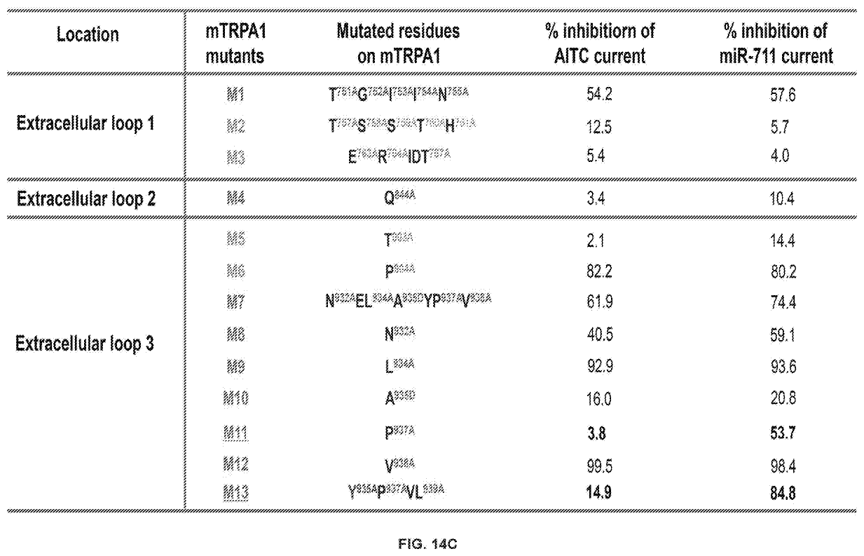

In order to explore the binding modes of miR-711 core sequence GGGACCC (SEQ ID NO: 1) to TRPA1 ion channel, we performed extensive replica exchange discrete molecular dynamics simulations (RexDMD) (Dokholyan et al., Fold. Des. 1998, 3, 577-587; Shirvanyants et al., J. Phys. Chem. B 2012, 116, 8375-8382), starting from the cryo-electron microscopy coordinates of TRPA1 (Paulsen et al., Nature 2015, 520, 511-517) and the structural model of GGGACCC generated using iFoldRNA (Sharma et al., Bioinformatics 2006, 22, 2693-2694). We estimated the binding energies between miRNA and TRPA1 along the entire simulations and collected the ensemble of high-affinity GGGACCC/TRPA1 conformations (i.e., energy lower or equal than −75 kcal/mol, FIG. 5A-FIG. 5D and FIG. 13A-FIG. 13C). The representative high-affinity and stable binding mode of GGGACCC to human TRPA1 complex is represented in FIG. 5A and FIG. 5B, while the frequencies of contacts between TRPA1 residues and GGGACCC motif are summarized in FIG. 5C. Because miR-711 has a shared core sequence in different species (FIG. S1D), we postulate that TRPA1 residues interacting with miR-711 should be conserved in mouse and human TRPA1 (mTRPA1 and hTRPA1). Sequence alignment of human, mouse, and rat TRPA1 showed that the predicated amino acid residues with possible interactions with GGGACCC are classified into three categories: non-conservative sites with different properties, conservative sites with similar properties, and ultra-conservative sites with identical amino acids (FIG. 14A). FIG. 5A shows the proximity of the GGGACCC core sequence with the Subunit 1, 2, and 3 of hTRPA1. The hTRPA1 residues interacting with individual nucleotides of GGGACCC were also highlighted in FIG. 5B and FIG. 13D-FIG. 13K, with special focus on P934 of hTRPA1 (FIG. 5B-FIG. 5C).

### Example 7

### miR-711 Activates mTRPA1 at P937 of the S5-S6 Extracellular Loop

AITC binds the intracellular ankyrin repeats at the N-terminal of TRPA1. We predicted that miR-711 might interact with TRPA1 at extracellular sites, given the hydrophilic nature of miRNAs. This prediction is also consistent with the result from computer simulation (FIG. 5A-FIG. 5D). According to the prediction in FIG. 5C and amino acid sequence alignment in FIG. 14A, we generated 8 mutations on the predicted and highly conserved sites in mTRPA1: one in S1-S2 loop (M1), one in S3-S4 loop (M4), and six in S5-S6 (M7, M8, M9, M10, M11, M12), which are illustrated in FIG. 14B and FIG. 14C. We also generated 4 mutations in the extracellular loop of mTRPA1 (M2, M3, M5, M6). In addition, we produced mutant M13, containing one mutation on predicted and nonconservative site Y⁹³⁶, one mutation on predicted and ultraconservative site P⁹³⁷, and one mutation on non-predicted site L⁹³⁹ (FIG. 14B-FIG. 14C). We excluded those mutations that caused marked disruption of TRPA1 structure and function based on loss of AITC responses. Notably, mutants M1, M6, M7, M8, M9, and M12 showed substantial reductions in both AITC and miR-711 induced currents (FIG. 5E, FIG. 14B, and FIG. 14C).

To assess the specific changes evoked by miR-711, we focused on the remaining TRPA1 mutants, in which AITC-induced currents were unaltered, including M2, M3, M4, M5, M10, M11, and M13 (FIG. 5E-FIG. 5G, FIG. 14B and FIG. 14C). Strikingly, miR-711-evoked currents were markedly reduced in M11 after a single residue mutation of mP937 (equivalent to hP934) at the S5-S6 extracellular loop (FIG. 5E-FIG. 5G, FIG. 14B, and FIG. 14C). Potential interactions of hP934 with nucleotides G003 and A004 are illustrated in FIG. 5B, FIG. 5C, and FIG. 13D-FIG. 13K. As expected, M13 with triple mutations (Y936, P937, and L939), including M11 single mutation at P937, resulted in further reduction in miR-711 current (FIG. 5E-FIG. 5G, FIG. 14B, and FIG. 14C), suggesting that the adjacent residues (Y936 and L939 of mTRPA1, equivalent to H933 and L936 of hTRPA1) may also interact with the core sequence to regulate TRPA1 function. In contrast, M2, M3, M4, M5, and M10 had no effects on either AITC or miR-711 induced currents (FIG. 14B-FIG. 14C). Collectively, these results show that extracellular residues, especially P937, can interact with the core sequence to regulate TRPA1 activation by miR-711.

### Example 8

### Interaction of miR-711 and TRPA1 is Required for miR-711 to Elicit Itch

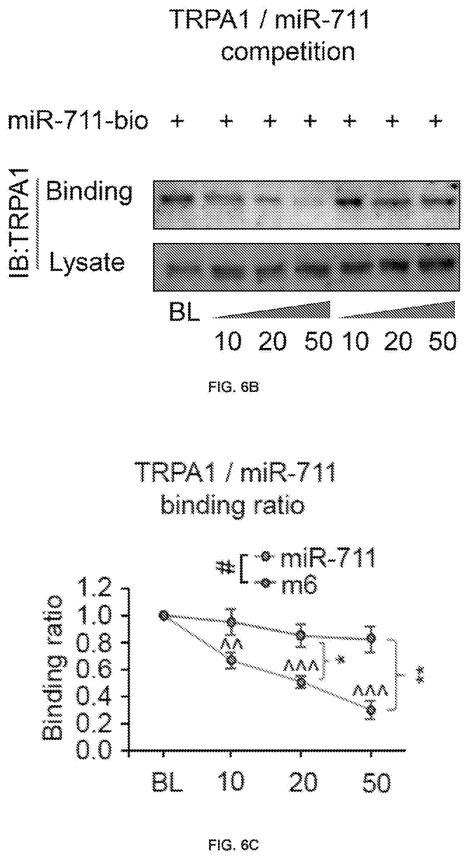

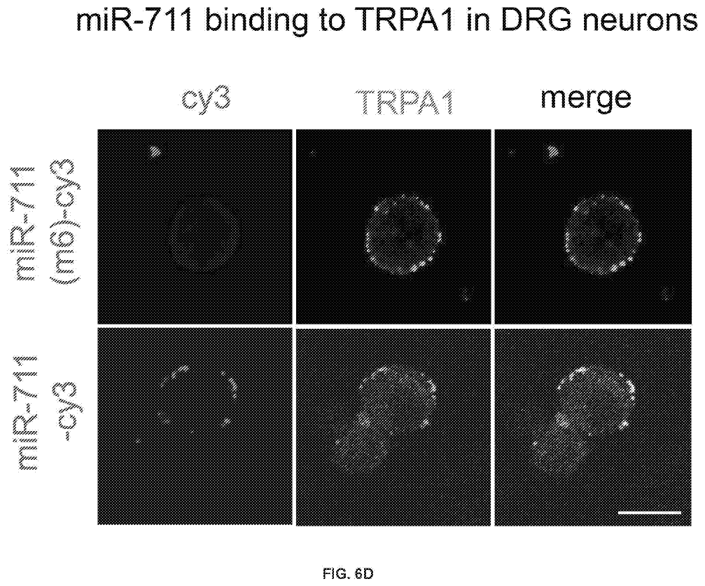

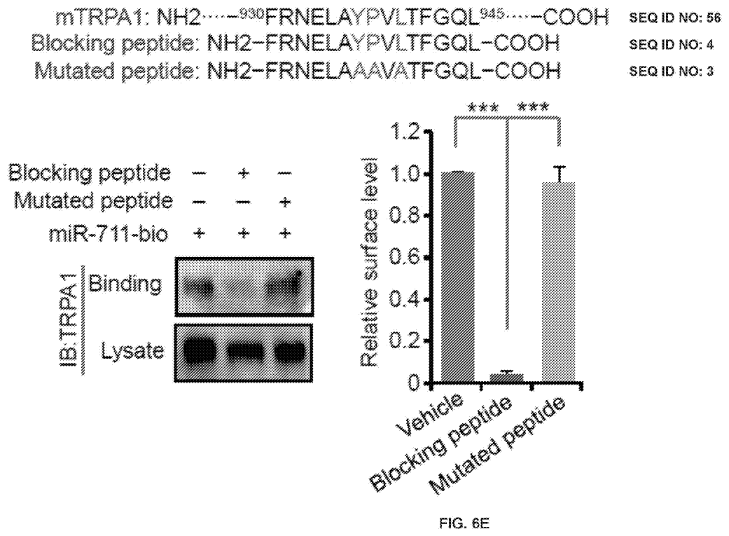

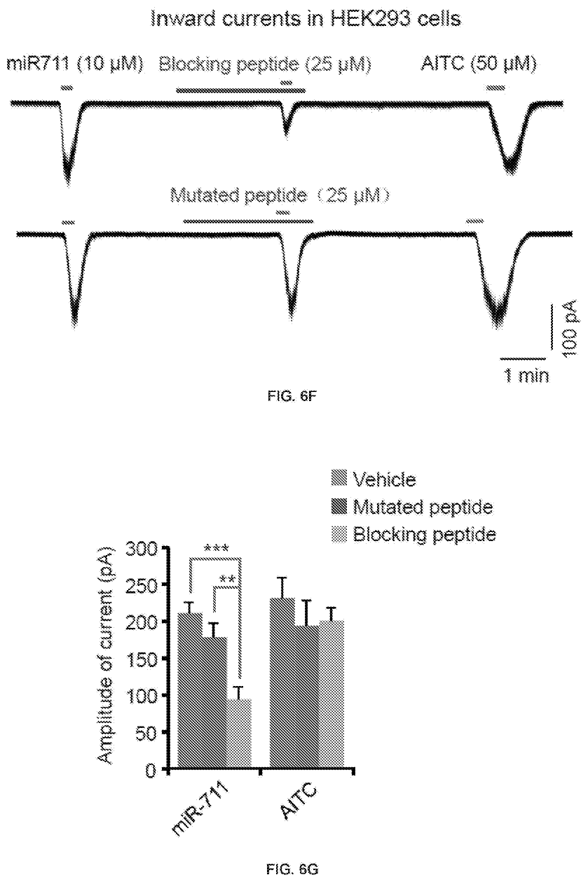

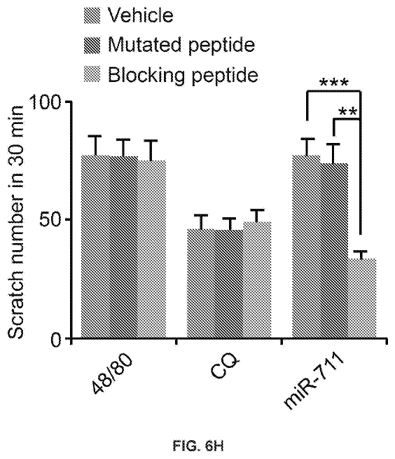

To assess the interaction of miR-711 and TRPA1, we also conducted an RNA binding assay using biotin-conjugated miR-711, which was able to pull down TRPA1 (FIG. 6A). By contrast, the mutant miR-711 (m6) only showed weak binding activity to TRPA1 (FIG. 6A) and failed to elicit itch (FIG. 1E). Competing experiment confirmed that wild-type but not mutant miR-711 (m6) inhibited the binding of biotin-labeled miR-711 to TRPA1 (FIG. 6B and FIG. 6C). We also tested the miRNA/TRPA1 interaction in native mouse neurons by incubating DRG neuronal cultures with Cy3-labeled miR-711. Immunofluorescence revealed that Cy3-labeled miR-711 but not Cy3-labeled mutant oligo (m6) binds to TRPA1 on the cell surface. However, TRPA1-negative DRG neurons show no binding to Cy3-labeled miR-711 (FIG. 6D).

To determine a physiological relevance of the miR-711/TRPA1 interaction, we investigated whether blocking the interaction would affect itch. To this end, we designed a small blocking peptide, FRNELAYPVLTFGQL (SEQ ID NO: 4), which covers the underlined residues Y936, P937, and L939 in the S5-S6 loop of mTRPA1, as well as other potentially interacting residues in Subunit-1 of TRPA1 (FIG. 5B and FIG. 5C). Notably, the blocking peptide disrupted the miR-711/TRPA1 interaction (FIG. 6E) and suppressed the miR-711-induced TRPA1 currents in Trpa1-expressing HEK293 cells (FIG. 6F and FIG. 6G). Importantly, intradermal injection of the blocking peptide prevented the miR-711-induced pruritus (FIG. 6H). In contrast, the blocking peptide did not affect the AITC-induced inward current (FIG. 6F and FIG. 6G) and had no effect on acute itch induced by compound 48/80 and chloroquine (FIG. 6H), implying that the effects of the blocking peptide are specific for miR-711. In a control experiment, we also designed a mutated peptide, with mutations in 3 residues underlined (FRNELAAAVATFGQL (SEQ ID NO: 3), FIG. 6E). Neither did this mutated peptide block the miR-711/TRPA1 interaction, nor did this peptide inhibit the miR-711-induced inward currents and pruritus (FIG. 6E-FIG. 6H). Taken together, our data demonstrate that the miR-711/TRPA1 interaction is critically involved in pruritus by miR-711.

### Example 9

### A Mouse Model of CTCL Exhibits Chronic Itch and miRNA Dysregulation

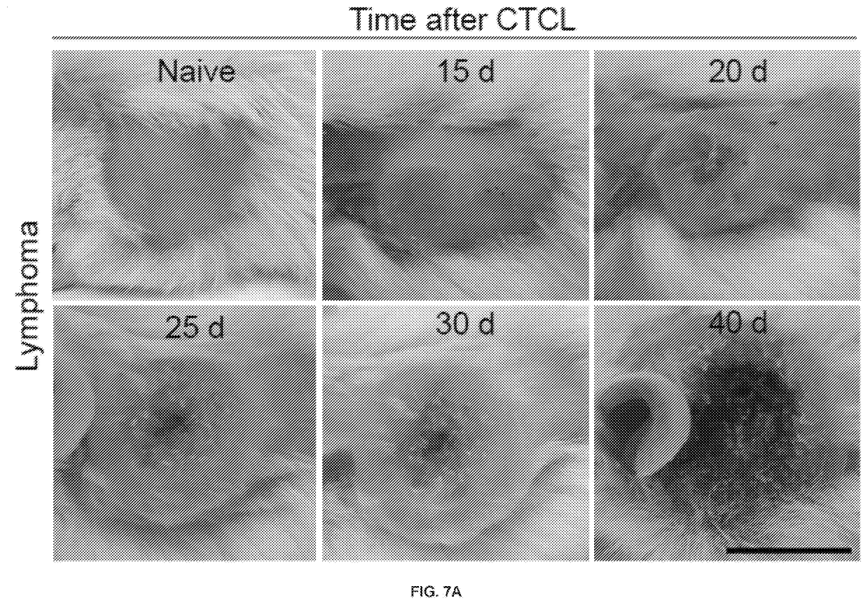

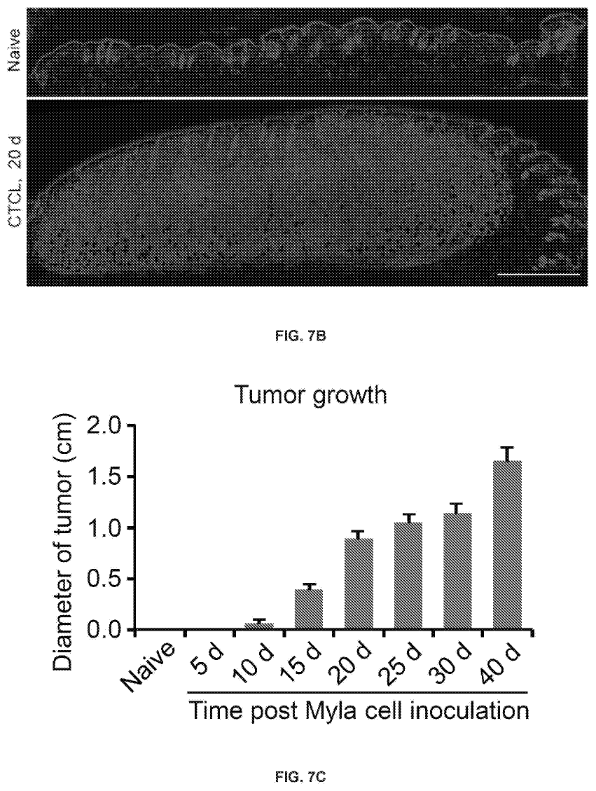

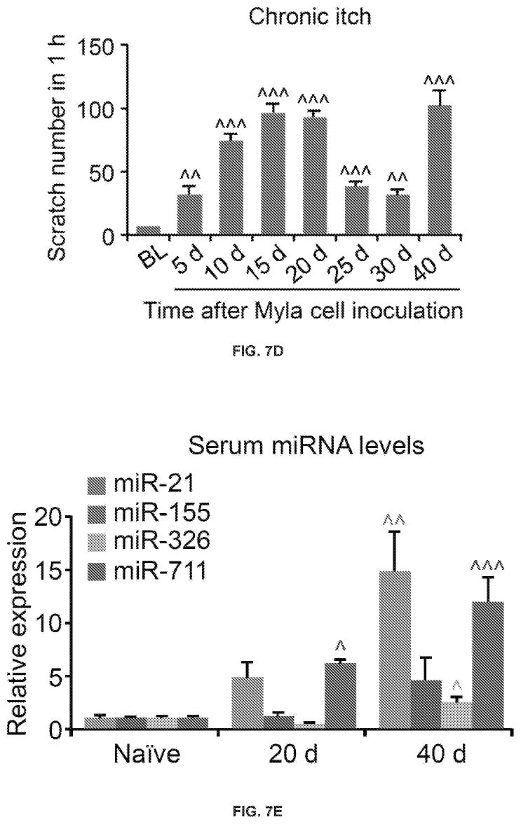

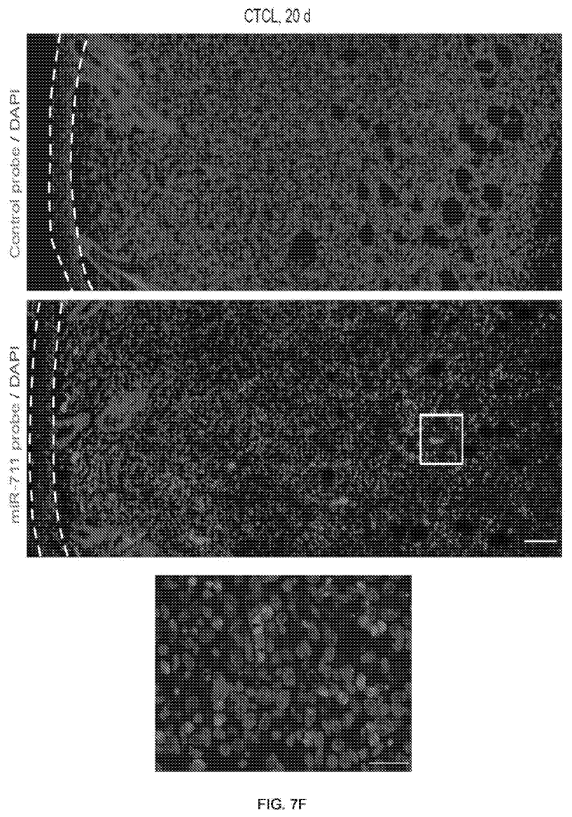

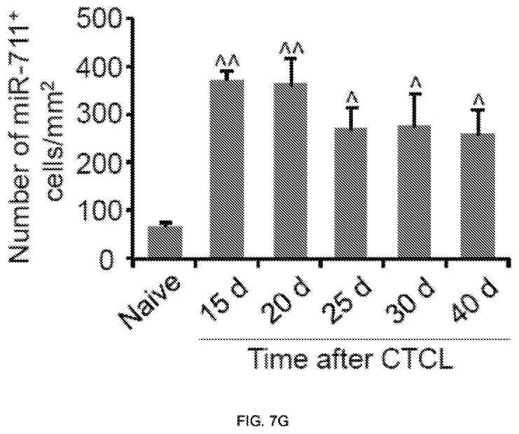

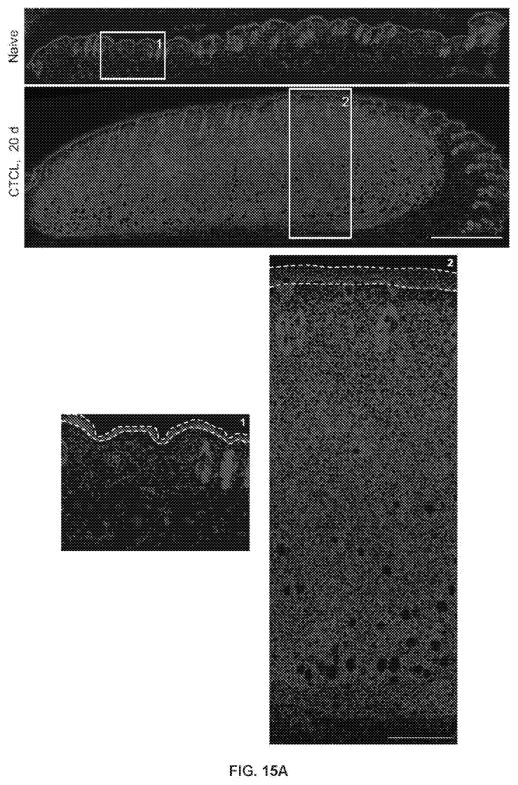

To further address the physiological and pathological relevance of miR-711 in chronic itch, we developed a murine xenograft model of chronic itch to recapitulate human symptoms of cutaneous T cell lymphoma (CTCL), using Myla cell line (CD4⁺ memory T cells) from a CTCL patient (Ralfkiaer et al., Blood 2011, 118, 5891-5900). Intradermal inoculation of Myla cells induced profound lymphoma on the back skin of immune-deficient scid mice, with a slow but persistent tumor growth; tumor was evident on day 15 and continued to grow on day 40 (FIG. 7A-FIG. 7C and FIG. 15A-FIG. 15B). Strikingly, this CTCL model was also characterized by an early onset of itch, prior to the onset of tumor growth: scratching behavior began on day 5 and reached to a peak on day 15 (FIG. 7D). This early onset of pruritus may result from pruritogen(s) secreted from the inoculated human cells. Notably, pruritus declined from the peak on day 25 and day 30 but returned to the peak level on Day 40 (FIG. 7D), suggesting a development of chronic itch. Mouse CTCL was also associated with increases in the thickness of epidermis (hypertrophy) and dermis with lymphoma progress (FIG. 15A-FIG. 15B).

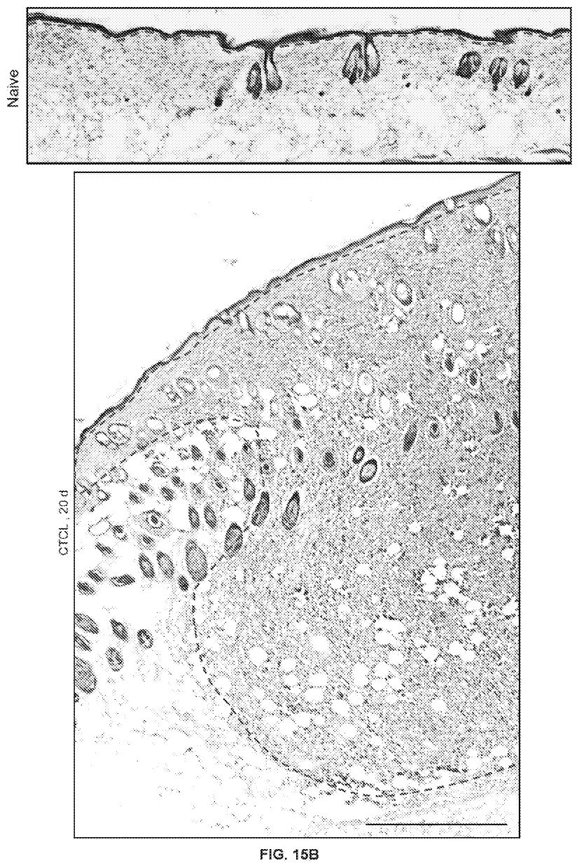

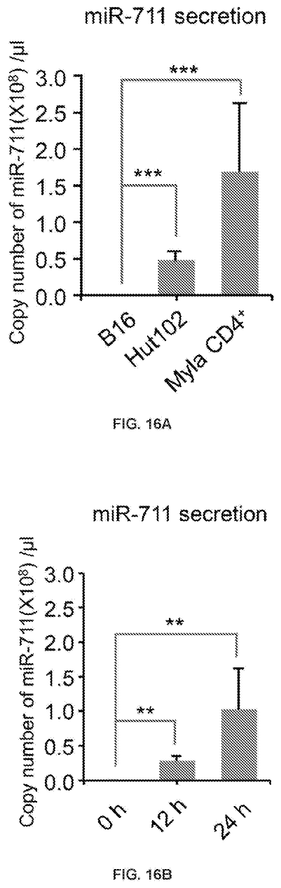

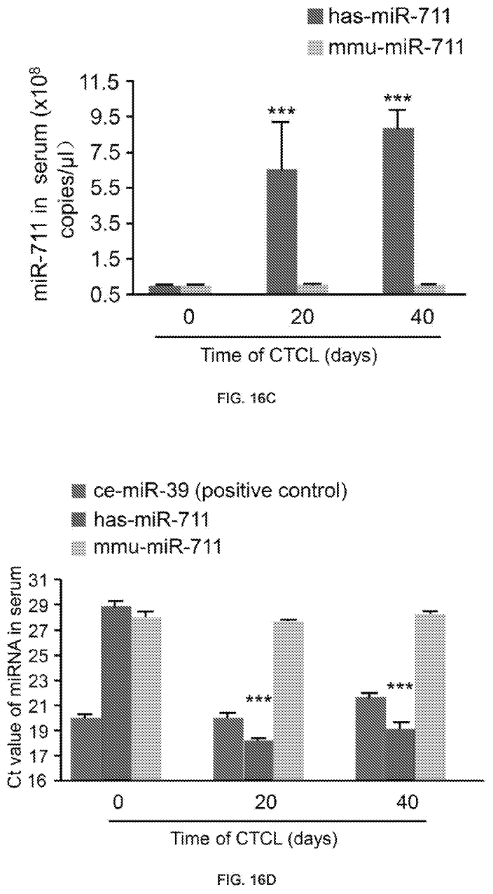

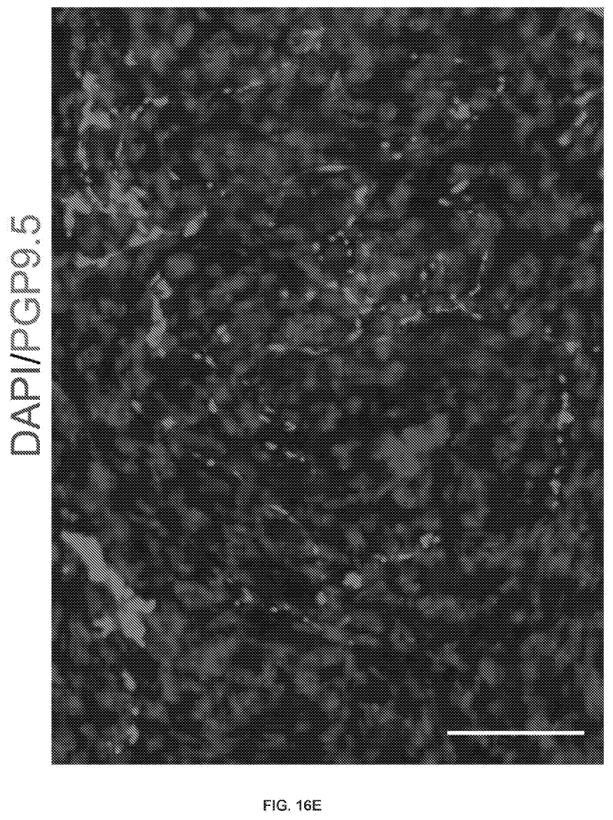

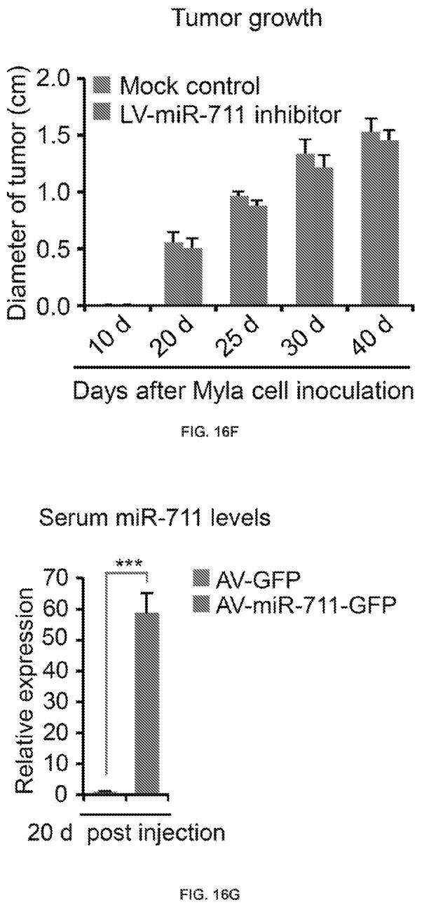

In parallel with dysregulations of miR-21, miR-155, miR-326, and miR-711 in CTCL patients, we found increased levels of hsa-miR-21, hsa-miR-155, hsa-miR-326, and hsa-miR-711 (FIG. 7E) in mouse serum, 20 and 40 days after murine CTCL. Since these are human miRNAs, they must be derived from inoculated human lymphoma cells. In situ hybridization demonstrated a persistent and broad expression of hsa-miR-711 in the back skin of CTCL mice (FIG. 7F and FIG. 7G). High levels of hsamiR-711 were also detected in the culture medium of Myla cells (z200 million copies per microliter) and Hut102 cells from another human lymphoma cell line, but not mouse B16 melanoma cells (FIG. 16A and FIG. 16B), further suggesting that hsamiR-711 can be secreted from human lymphoma cells. Notably, mouse- and human-derived miR-711, mmu-miR-711, and hsamiR-711 differ in two nucleotides but share the same core sequence (FIG. 1C). qPCR analysis detected high copy numbers of hsa-miR-711 but very low copy numbers of mmumiR-711 in serum samples of CTCL mice, and a similar result was obtained when the data were plotted as Ct values (FIG. 16C and FIG. 16D). These data suggest that (1) qPCR is highly specific to distinguish human versus mouse miR-711 and (2) miR-711 in mouse serum is predominantly derived from inoculated human lymphoma cells. Strikingly, skin lymphoma was innervated by skin nerve fibers labeled with PGP-9.5 (FIG. 16E). Thus, tumor-released miR-711 could trigger pruritus by activating adjacent nerve fibers which express TRPA1. These itch-inducing nerve fibers could be present in the tumor or nearby epidermis.

### Example 10

### miR-711 Regulates Chronic Itch after CTCL

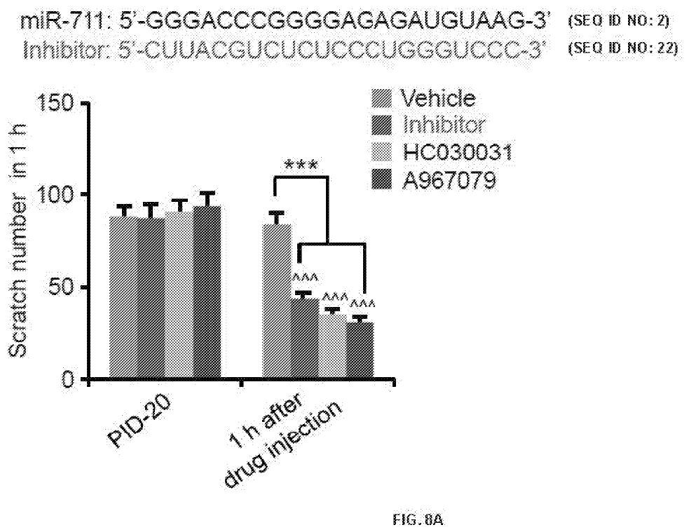

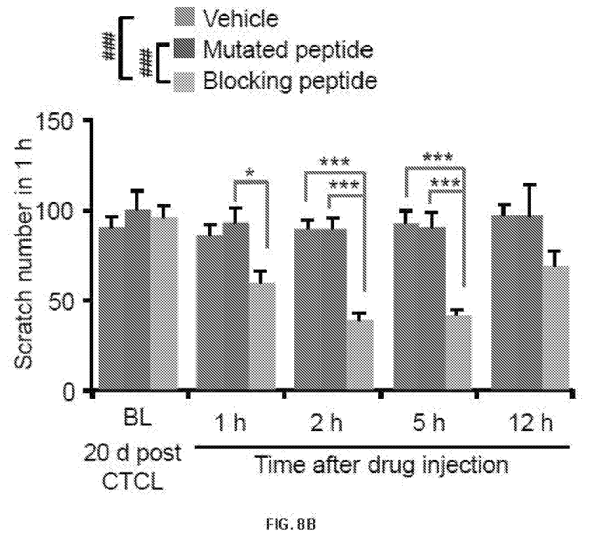

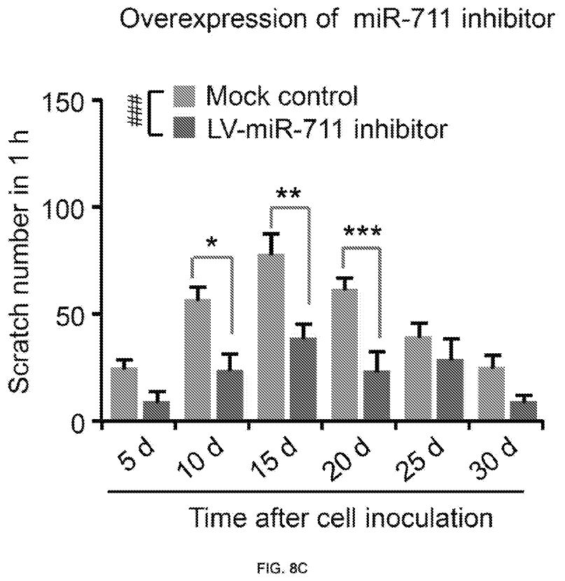

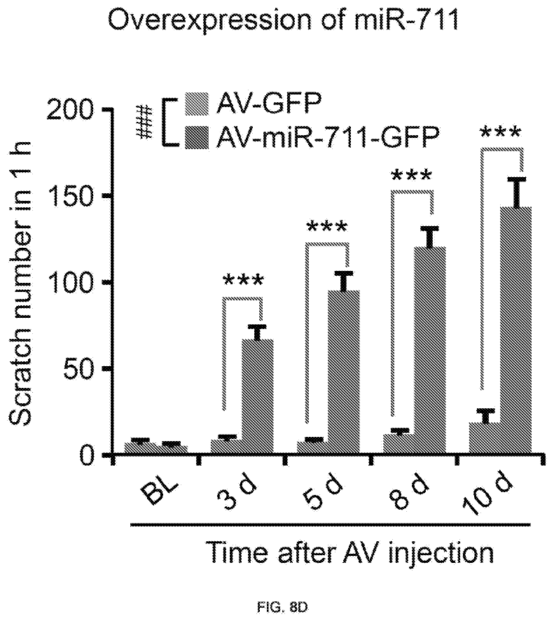

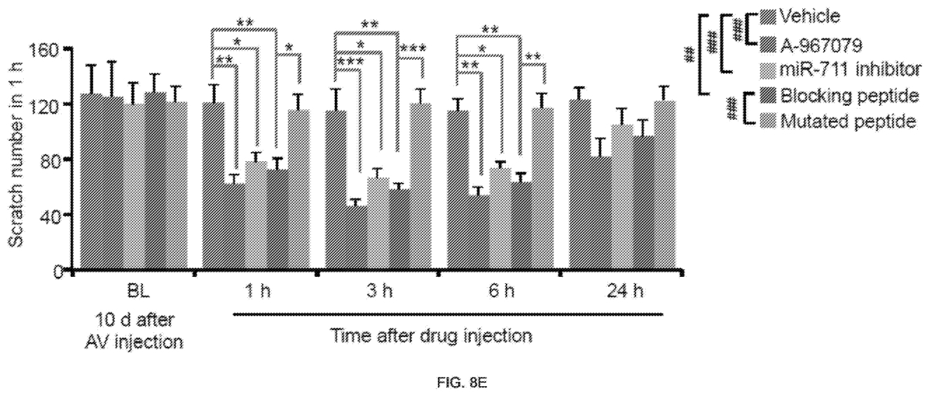

To determine a role of miR-711 in chronic itch, we employed several pharmacological and genetic approaches to target miR-711. First, intratumoral injection of a miR-711 inhibitor with a complementary sequence to hsa-miR-711 (FIG. 8A), given 20 days after Myla cell inoculation, reduced chronic pruritus (FIG. 8A). Second, CTCL-induced chronic itch was suppressed by two structurally different TRPA1 antagonists, HC30031 and A967079 (FIG. 8A). Third, disruption of the miR-711 and TRPA1 interaction with the blocking peptide but not mutated peptide, given 20 days after the Myla cell inoculation, effectively reduced the CTCL-evoked chronic itch for more than 5 hr (FIG. 8B). Fourth, to achieve a sustained inhibition of miR-711, we generated a stable cell line expressing hsa-miR-711 inhibitor in Myla cells before the inoculation. Overexpression of the miR-711 inhibitor via lentivirus (LV) delayed the development of chronic itch for 20 days (FIG. 8C), without affecting the tumor growth (FIG. 16F). Fifth, overexpression of miR-711 via adenovirus (AV) resulted in persistent itch for more than 10 days and increased serum levels of hsa-miR-711 (FIG. 8D and FIG. 16G), indicating that sustained miR-711 increase in serum is associated with chronic itch. Finally, we confirmed that chronic itch, evoked by miR-711 overexpression via AV, is mediated by miR-711, TRPA1, and the miR-711/TRPA1 interaction, because the AV-induced pruritus was suppressed by the miR-711 inhibitor, the TRPA1 antagonist, and the blocking peptide (FIG. 8E). Taken together, these loss-of-function and gain-of-function experiments demonstrated that miR-711 is critically involved in chronic itch through TRPA1.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A method of treating a disease or condition in a subject, the method comprising administering to the subject a miR-711 inhibitor.

Clause 2. A method of inhibiting TRPA1 in a subject, the method comprising administering to the subject a miR-711 inhibitor.

Clause 3. A method of inhibiting miR-711 in a subject, the method comprising administering to the subject a miR-711 inhibitor selected from a miR-711/TRPA1 interaction blocking peptide, a polynucleotide complementary to miR-711, or a combination thereof.

Clause 4. The method of any one of clauses 1-2, wherein the miR-711 inhibitor is selected from a miR-711/TRPA1 interaction blocking peptide, a polynucleotide complementary to miR-711, or a combination thereof.

Clause 5. The method of clause 3 or 4, wherein the miR-711/TRPA1 interaction blocking peptide comprises a polypeptide having an amino acid sequence of SEQ ID NO: 3 (FRNELAAAVATFGQL).

Clause 6. The method of clause 3 or 4, wherein the miR-711/TRPA1 interaction blocking peptide comprises a polypeptide having an amino acid sequence of SEQ ID NO: 4 (FRNELAYPVLTFGQL).

Clause 7. The method of any one of clauses 3-4, wherein the miR-711 inhibitor comprises a polynucleotide complementary to miR-711 or a portion or fragment thereof.

Clause 8. The method of any one of clauses 1-7, the method further comprising additionally administering a TRPA1 inhibitor.

Clause 9. The method of clause 8, wherein the TRPA1 inhibitor is selected from HC030031 or A967079, or a pharmaceutically acceptable salt thereof.

Clause 10. The method of any one of clauses 1 and 4-9, wherein the disease or condition is selected from pruritis, atopic eczema, and psoriasis.

Clause 11. The method of clause 10, wherein the pruritis is chronic pruritis.

Clause 12. The method of clause 10, wherein the pruritis is acute pruritis.

Clause 13. The method of clause 10, wherein the pruritis is lymphoma-induced pruritis.

Clause 14. The method of clause 10, wherein the pruritis is pruritis associated with lymphoma.

Clause 15. The method of clause 10, wherein the pruritis is pruritis associated with liver disease.

Clause 16. The method of any one of clauses 1-15, wherein miR-711 comprises a core polynucleotide sequence of SEQ ID NO: 1.

Clause 17. The method of any one of clauses 1-16, wherein the miR-711 inhibitor inhibits nerve fibers expressing TRPA1.

Clause 18. The method of any one of clauses 1-16, wherein the binding of miR-711 to the extracellular side of TRPA1 is inhibited.

Clause 19. The method of clause 18, wherein the binding of miR-711 to TRPA1 at S5-S6 loop is inhibited.

Clause 20. The method of clause 18, wherein the binding of miR-711 to TRPA1 at an amino acid corresponding to P934 of human TRPA1 (SEQ ID NO: 55) is inhibited.

Clause 21. A composition comprising a miR-711 inhibitor, wherein the miR-711 inhibitor is selected from a miR-711/TRPA1 interaction blocking peptide, a polynucleotide complementary to miR-711, or a combination thereof.

Clause 22. The composition of clause 21, wherein the miR-711/TRPA1 interaction blocking peptide comprises a polypeptide having an amino acid sequence of SEQ ID NO: 3 (FRNELAAAVATFGQL) or SEQ ID NO: 4 (FRNELAYPVLTFGQL).

Clause 23. The composition of clause 21 or 22, wherein the composition further comprises a TRPA1 inhibitor.

Clause 24. The composition of clause 23, wherein the TRPA1 inhibitor is selected from HC030031 or A967079, or a pharmaceutically acceptable salt thereof.

SEQUENCES
Core polynucleotide sequence of miR-711
SEQ ID NO: 1
GGGACCC
Full polynucleotide sequence of miR-711
SEQ ID NO: 2
GGGACCCGGGGAGAGAUGUAAG
miR-711/TRPA1 interaction blocking peptide
SEQ ID NO: 3
FRNELAAAVATFGQL
miR-711/TRPA1 interaction blocking peptide
SEQ ID NO: 4
FRNELAYPVLTFGQL
mmu-miR-711
SEQ ID NO: 5
gggacccggggagagauguaag
mmu-miR-21
SEQ ID NO: 6
uagcuuaucagacugauguuga
mmu-miR-155
SEQ ID NO: 7
uuaaugcuaauugugauaggggu
mmu-miR-326
SEQ ID NO: 8
gggggcagggccuuugugaaggcg
hsa-miR-711
SEQ ID NO: 9
gggacccagggagagagacguaag
hsa-miR-642b-3p
SEQ ID NO: 10
agacacauuuggagagggaccc
mmu-miR-711(m1)
SEQ ID NO: 11
aaaacccggggagagauguaag
mmu-miR-711(m2)
SEQ ID NO: 12
gggaaaaggggagagauguaag
mmu-miR-711(m3)
SEQ ID NO: 13
gggacccgaaaagagauguaag
mmu-miR-711(m4)
SEQ ID NO: 14
gggacccggggaaaaauguaag
mmu-miR-711(m5)
SEQ ID NO: 15
gggacccggggagagauaaaag
mmu-miR-711(m6)
SEQ ID NO: 16
aaaaaaaggggagagauguaag
Mmu-miR-711 mutant
SEQ ID NO: 17
aaaaaaa
mmu-miR-711-bio
SEQ ID NO: 18
gggacccggggagagauguaag-bio
mmu-miR-711(m6)-bio
SEQ ID NO: 19
aaaaaaaggggagagauguaag-bio
mmu-miR-711-cy3
SEQ ID NO: 20
gggacccggggagagauguaag-cy3
mmu-miR-711(m6)-cy3
SEQ ID NO: 21
aaaaaaaggggagagauguaag-cy3
hsa-miR-711 inhibitor
SEQ ID NO: 22
cuuacgucucucccuggguccc
(DIG)-labeled miRCURY LNA ™ Detection
probe against hsa-miR-711
SEQ ID NO: 23
cttacgtctctccctgggtc
(DIG)-labeled miRCURY LNA ™ Detection
negative control probe
SEQ ID NO: 24
gtgtaacacgtctatacgccca
Forward primer for subcloning mTrpa1
into pcgn
SEQ ID NO: 25
5'-agcctgggaggaccttctagaatgaagcgcggcttgagg-3'
Reverse primer for subcloning mTrpa1
into pcgn
SEQ ID NO: 26
5'-ctcaccctgaagttctcaggatccctaaaagtccgggtggc-3'
Reverse primer for mTrpa1 N-terminal
deletion
SEQ ID NO: 27
5'-ctcaccctgaagttctcaggatccctaaaagtccgggtggc-3'
Forward primer for PCGN- mTrpa1 (M1)
SEQ ID NO: 28
5'-gctgcagcagccgctggaactagtagtac-3'
Reverse primer for PCGN- mTrpa1 (M1)
SEQ ID NO: 29
5'-agaattgaaggccattccag-3'
Forward primer for PCGN- mTrpa1 (M2)
SEQ ID NO: 30
5'-aattctgctggaataatcgctggaactag-3'
Reverse primer for PCGN- mTrpa1 (M2)
SEQ ID NO: 31
5'-attattccagtagaattgaaggcc-3'
Forward primer for PCGN- mTrpa1 (M3)
SEQ ID NO: 32
5'-atgaggcagcaatagacgctctgaattcatttcca-3'
Reverse primer for PCGN- mTrpa1 (M3)
SEQ ID NO: 33
5'-gagtactactagttccattgattattc-3'
Forward primer for PCGN- mTrpa1 (M4)
SEQ ID NO: 34
5'-tatatggcgtggcaatgtggag-3'
Reverse primer for PCGN- mTrpa1 (M4)
SEQ ID NO: 35
5'-cgctgggatgttgaggaacaag-3'
Forward primer for PCGN- mTrpa1 (M5)
SEQ ID NO: 36
5'-gccccattgctttccttaatcc-3'
Forward primer for PCGN- mTrpa1 (M5)
SEQ ID NO: 37
5'-gctgaaggcatcttggaaattc-3'
Forward primer for PCGN- mTrpa1 (M6)
SEQ ID NO: 38
5'-agcaccgcattgctttccttaatc-3'
Reverse primer for PCGN- mTrpa1 (M6)
SEQ ID NO: 39
5'-gaaggcatcttggaaattc-3'
Forward primer for PCGN- mTrpa1 (M7)
SEQ ID NO: 40
5'-gctgaggcggaatacgcagccctgacctttg-3'
Forward primer for PCGN- mTrpa1 (M8)
SEQ ID NO: 41
5'-gtttagagctgagttggcatac-3'
Forward primer for PCGN- mTrpa1 (M9)
SEQ ID NO: 42
5'-gtttagaaatgaggcggcatac-3'
Reverse primer for PCGN- mTrpa1 (M8),
PCGN-Trpa1(M9)
SEQ ID NO: 43
5'-aatggttctaggaaggcatctc-3'
Forward primer for PCGN- mTrpa1 (M10)
SEQ ID NO: 44
5'-aatgagttggaatacccagtcctg-3'
Forward primer for PCGN- mTrpa1 (M11)
SEQ ID NO: 45
5'-aatgagttggcatacgcagtcctg-3'
Forward primer for PCGN- mTrpa1 (M12)
SEQ ID NO: 46
5'-aatgagttggcatacccagccctg-3'
Reverse primer for PCGN- mTrpa1 (M7),
PCGN-Trpa1(M10), PCGN-Trpa1(M11),
PCGN-Trpa1(M12)
SEQ ID NO: 47
5'-tctaaacaatggttctaggaag-3'
Forward primer for PCGN- mTrpa1 (M13)
SEQ ID NO: 48
5'-gttggca gccgcagtcgcgacctttgggcagc-3'
Reverse primer for PCGN- mTrpa1 (M13)
SEQ ID NO: 49
5'-tcatttctaaacaatggttctag-3'
eca-miR-711
SEQ ID NO: 50
gggacccagggagagagacguaag
mml-miR-711
SEQ ID NO: 51
gggacccagggagagagacguaag
ptr-miR-711
SEQ ID NO: 52
gggacccagggagagagacguaag
mmu-miR-711
SEQ ID NO: 53
gggacccggggagagagauguaag
rno-miR-711
SEQ ID NO: 54
gggacccugggagagagauguaag
TRPA1 polypeptide sequence (human)
hTRPA1
SEQ ID NO: 55
MMNLGSYCLGLIPMTILVVNIKPGMAFNSTGIINETSDHSEILDTTNSYL
IKTCMILVFLSSIFGYCKEAGQIFQQKRNYFMDISNVLEWIIYTTGIIFV
LPLFVEIPAHLQWQCGAIAVYFYWMNFLLYLQRFENCGIFIVMLEVILKT
LLRSTVVFIFLLLAFGLSFYILLNLQDPFSSPLLSIIQTFSMMLGDINYR
ESFLEPYLRNELAHPVLSFAQLVSFTIFVPIVLMNLLIGLAV
TRPA1 polypeptide sequence (mouse)
mTRPA1
SEQ ID NO: 56
AHMMNLGSYCLGLIPMTLLVVKIQPGMAFNSTGIINGTSSTHEERIDTLN
SFPIKICMILVFLSSIFGYCKEVIQIFQQKRNYFLDYNNALEWVIYTTSI
IFVLPLFLNIPAYMQWQCGAIAIFFYWMNFLLYLQRFENCGIFIVMLEVI
FKTLLRSTGVFIFLLLAFGLSFYVLLNFQDAFSTPLLSLIQTFSMMLGDI
NYRDAFLEPLFRNELAYPVLTFGQLIAFTMFVPIVLMNLLIGLAV
TRPA1 polypeptide sequence (rat)
rTRPA1
SEQ ID NO: 57
AHMMNLGSYCLGLIPMTLLVVKIQPGMAFNSTGIINETISTHEERINTLN
SFPLKICMILVFLSSIFGYCKEVVQIFQQKRNYFLDYNNALEWVIYTTSM
IFVLPLFLDIPAYMQWQCGAIAIFFYWMNFLLYLQRFENCGIFIVMLEVI
FKTLLRSTGVFIFLLLAFGLSFYVLLNFQDAFSTPLLSLIQTFSMMLGDI
NYRDAFLEPLFRNELAYPVLTFGQLIAFTMFVPIVLMNLLIGLAV

1. A method of treating a disease or condition in a subject, the method comprising administering to the subject a miR-711 inhibitor.

2. A method of inhibiting TRPA1 in a subject, the method comprising administering to the subject a miR-711 inhibitor.

3. A method of inhibiting miR-711 in a subject, the method comprising administering to the subject a miR-711 inhibitor selected from a miR-711/TRPA1 interaction blocking peptide, a polynucleotide complementary to miR-711, or a combination thereof.

4. The method of any one of claims 1-2, wherein the miR-711 inhibitor is selected from a miR-711/TRPA1 interaction blocking peptide, a polynucleotide complementary to miR-711, or a combination thereof.

5. The method of claim 3 or 4, wherein the miR-711/TRPA1 interaction blocking peptide comprises a polypeptide having an amino acid sequence of SEQ ID NO: 3 (FRNELAAAVATFGQL).

6. The method of claim 3 or 4, wherein the miR-711/TRPA1 interaction blocking peptide comprises a polypeptide having an amino acid sequence of SEQ ID NO: 4 (FRNELAYPVLTFGQL).

7. The method of any one of claims 3-4, wherein the miR-711 inhibitor comprises a polynucleotide complementary to miR-711 or a portion or fragment thereof.

8. The method of any one of claims 1-7, the method further comprising additionally administering a TRPA1 inhibitor.

9. The method of claim 8, wherein the TRPA1 inhibitor is selected from HC030031 or A967079, or a pharmaceutically acceptable salt thereof.

10. The method of any one of claims 1 and 4-9, wherein the disease or condition is selected from pruritis, atopic eczema, and psoriasis.

11. The method of claim 10, wherein the pruritis is chronic pruritis.

12. The method of claim 10, wherein the pruritis is acute pruritis.

13. The method of claim 10, wherein the pruritis is lymphoma-induced pruritis.

14. The method of claim 10, wherein the pruritis is pruritis associated with lymphoma.

15. The method of claim 10, wherein the pruritis is pruritis associated with liver disease.

16. The method of any one of claims 1-15, wherein miR-711 comprises a core polynucleotide sequence of SEQ ID NO: 1.

17. The method of any one of claims 1-16, wherein the miR-711 inhibitor inhibits nerve fibers expressing TRPA1.

18. The method of any one of claims 1-16, wherein the binding of miR-711 to the extracellular side of TRPA1 is inhibited.

19. The method of claim 18, wherein the binding of miR-711 to TRPA1 at S5-S6 loop is inhibited.

20. The method of claim 18, wherein the binding of miR-711 to TRPA1 at an amino acid corresponding to P934 of human TRPA1 (SEQ ID NO: 55) is inhibited.

21. A composition comprising a miR-711 inhibitor, wherein the miR-711 inhibitor is selected from a miR-711/TRPA1 interaction blocking peptide, a polynucleotide complementary to miR-711, or a combination thereof.

22. The composition of claim 21, wherein the miR-711/TRPA1 interaction blocking peptide comprises a polypeptide having an amino acid sequence of SEQ ID NO: 3 (FRNELAAAVATFGQL) or SEQ ID NO: 4 (FRNELAYPVLTFGQL).

23. The composition of claim 21 or 22, wherein the composition further comprises a TRPA1 inhibitor.

24. The composition of claim 23, wherein the TRPA1 inhibitor is selected from HC030031 or A967079, or a pharmaceutically acceptable salt thereof.
